SEQUENCE LISTING (89 sequences)

SEQ ID NO: 1 - DNA, 1647 bp, Bos taurus
  source 1..1647: mol_type = other DNA
ggtgcttacgaactgtaggtttctgtgcctttccccctagcttttgaagagcagagaggaagatggagaccgagtccggggcgcaaacatcaaatcagacgcaaacagattcattatctccatcccctaatcctgtgtcacctgtgcctttgaataacccaacgagtgccccaagatatggaacagtgatccctaatcgcatctttgtaggaggaattgactttaagacaaatgaaaatgatttaagaaaatttttttcccagtatggatctgtaaaagaagtgaagattgtaaatgacagagctggagtttccaaagggtatggcttcgtcacttttgaaacacaagaagatgcacaaaaaattttacaagaggctgaaaaacttaattacaaggataagaaactgaacattggcccggcaataagaaagcaacaagtagggatccctcgttccagtataatgccagcagctggaacaatgtatctaacaacttcaactggatatccttacacttatcataatggtgttgcttattttcatactccagaggttacttctgtcccaccaccttggccttcacgttctatatctagttcacctgtgatggtagctcagcctgtttatcaacaacctgcatatcactaccaggcccctgcacagtgtctaccaggtcaatggcagtggaatgttcctcagtctcctgcctcttctgctccattcttatacctacaaccttctgaggtcatttatcaaccagtggaaattgcacaagatggtggatgtgttcctcctccgctgtctctgatggaagcttcagttccagagccttattctgatcatggagttcaagcaacatatcaccaggtttatgcttcaagtgccattgctatgcctgcaccagtgatgcaacctgaaccaattaaaacagtgtggagcattcattattaagacaatttgggcagctctagtccagctcaactgatttcttatccagtgatccttgtgtagccgagatccagcttcaatgaaccggctagaagctgcccattgtgaaacttagggttagttaatacctcaccatacttatttattccactataagctgtctaaatttgatgaaatttgctttttcagctgttctacaaaattatttaggtaaatgtggcatgttgattttttaatgtgtttatctaactgtagtgacattttctacaacgtgttttatccattccataaataataaccacattgggaatagtgaggtgtctttcattttctctgctttgtgttatttcttttcctctcaatcttttagaagtttatgtttcatgttctttggtatttattttaatatttaggataagagtcatttctaattctaatgccttttatcatatagttaatcttaatgaatcagttctactgcttggcaattttgtcatttttgtctaatttcttgttactactgctctttaattttgcttctaccatggttttcagaattttaatagtatttatatgcatttttaaaaacatttccaaacgttagaaataccatcaacatgatcagtatttttgccttttcatgtgattaatttgactatattaaattttttctcctaacatttgagataatgctccagta

SEQ ID NO: 2 - DNA, 941 bp, Bos taurus
  source 1..941: mol_type = other DNA
gcttacgaactgtaggtttctgtgcctttccccctagcttttgaagagcagagaggaagatggagaccgagtccggggcgcaaacatcaaatcagacgcaaacagattcattatctccatcccctaatcctgtgtcacctgtgcctttgaataacccaacgagtgccccaagatatggaacagtgatccctaatcgcatctttgtaggaggaattgactttaagacaaatgaaaatgatttaagaaaatttttttcccagtatggatctgtaaaagaagtgaagattgtaaatgacagagctggagtttccaaagggtatggcttcgtcacttttgaaacacaagaagatgcacaaaaaattttacaagaggctgaaaaacttaattacaaggataagaaactgaacattggcccggcaataagaaagcaacaagtagggatccctcgttccagtataatgccagcagctggaacaatgtatctaacaacttcaactggatatccttacacttatcataatggtgttgcttattttcatactccagaggttacttctgtcccaccaccttggccttcacgttctatatctagttcccctgtgatggtagctcagcctgtttatcaacaacctgcatatcactaccaggcccctgcacagtgtctaccaggtcaatggcagtggaatgttcctcagtctcctgcctcttctgctccattcttatacctacaaccttctgaggtcatttatcaaccagtggaaattgcacaagatggtggatgtgttcctcctccgctgtctctgatggaagcttcagttccagagccttattctgatcatggagttcaagcaacatatcaccaggtttatgcttcaagtgccattgctatgcctgcaccagtgatgcaacctgaaccaattaaagtaagaagcttgttttga

SEQ ID NO: 3 - DNA, 1952 bp, Bos taurus
  source 1..1952: mol_type = other DNA
tggcggctacggcggcggtggcagctgcaagcggggggggagaaagtctccgtcgggaagcgtggtgttgcttcctgggtgtgaaggtgcttacgaactgtaggtttctgtgcctttccctctagcttttgaagagcagagaggaagatggagaccgagtccggggcgcaaacatcaaatcagacgcaaacagattcattatctccatcccctaatcctgtgtcacctgtgcctttgaataacccaacgagtgccccaagatatggaacagtgatccctaatcgcatctttgtaggaggaattgactttaagacaaatgaaaatgatttaagaaaatttttttcccagtatggatctgtaaaagaagtgaagattgtaaatgacagagctggagtttccaaagggtatggcttcgtcacttttgaaacacaagaagatgcacaaaaaattttacaagaggctgaaaaacttaattacaaggataagaaactgaacattggcccggcaataagaaagcaacaagtagggatccctcgttccagtataatgccagcagctggaacaatgtatctaacaacttcaactggatatccttacacttatcataatggtgttgcttattttcatactccagaggttacttctgtcccaccaccttggcctgcccctgcacagtgtctaccaggtcagtggcagtggaatgttcctcagtctcctgcctcttctgctccattcttatacctacaaccttctgaggtcatttatcaaccagtggaaattgcacaagatggtggatgtgttcctcctctgctgtctctgatggaagcttcagttccagagccttattctgatcatggagttcaagcaacatatcaccaggtttatgcttcaagtgccattgctatgcctgcaccagtgatgcaacctgaaccaattaaaacagtgtggagcattcattattaagacaatttgggcagctctagtccagctcaactgatttcttatccagtgatccttgtgtagccgagatccagcttcaatgaaccggctagaagctgcccattgtgaaacttagggttagttaatacctcaccatacttatttattccactataagctgtctaaatttgatgaaatttgctttttcagctgttctacaaaattatttaggtaaatgtggcatgttgattttttaatgtgtttatctaactgtagtgacattttctacaacgtgttttatccattccataaataataaccacattgggaatagtgaggtgtctttcattttctctgctttgtgttatttcttttcctctcaatcttttagaagtttatgtttcatgttctttggtatttattttaatatttaggataagagtcatttctaattctaatgccttttatcatatagttaatcttaatgaatcagttctactgcttggcaattttgtcatttttgtctaatttcttgttactactgctctttaattttgcttctaccatggttttcagaattttaatagtatttatatgcatttttaaaaacatttccaaacgttagaaataccatcaacatgatcagtatttttgccttttcatgtgattaatttgactatattaaattttttctcctaacatttgagataatgctccagtatccctataacataagccatttggtaccaaactttatataatatggatcacttagtagaattgttttgattaaaagaagggcttacctctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaaaaaaaaaaaaaaaa

SEQ ID NO: 4 - DNA, 1988 bp, Bos taurus
  source 1..1988: mol_type = other DNA
tggcggctacggcggcggtggcagctgcaagcggggggggagaaagtctccgtcgggaagcgtggtgttgcttcctgggtgtgaaggtgcttacgaactgtaggtttctgtgcctttccccctagcttttgaagagcagagaggaagatggagaccgagtccggggcgcaaacatcaaatcagacgcaaacagattcattatctccatcccctaatcctgtgtcacctgtgcctttgaataacccaacgagtgccccaagatatggaacagtgatccctaatcgcatctttgtaggaggaattgactttaagtatggatctgtaaaagaagtgaagattgtaaatgacagagctggagtttccaaagggtatggcttcgtcacttttgaaacacaagaagatgcacaaaaaattttacaagaggctgaaaaacttaattacaaggataagaaactgaacattggcccggcaataagaaagcaacaagtagggatccctcgttccagtataatgccagcagctggaacaatgtatctaacaacttcaactggatatccttacacttatcataatggtgttgcttattttcatactccagaggttacttctgtcccaccaccttggccttcacgttctatatctagttcacctgtgatgggagctcagcctgtttatcaacaacctgcatatcactaccaggcccctgcacagtgtctaccaggtcaatggcagtggaatgttcctcagtctcctgcctcttctgctccattcttatacctacaaccttctgaggtcatttatcaaccagtggaaattgcacaagatggtggatgtgttcctcctccgctgtctctgatggaagcttcagttccagagccttattctgatcatggagttcaagcaacatatcaccaggtttatgcttcaagtgccattgctatgcctgcaccagtgatgcaacctgaaccaattaaaacagtgtggagcattcattattaagacaatttgggcagctctagtccagctcaactgatttcttatccagtgatccttgtgtagccgagatccagcttcaatgaaccggctagaagctgcccattgtgaaacttagggttagttaatacctcaccatacttatttattccactataagctgtctaaatttgatgaaatttgctttttcagctgttctacaaaattatttaggtaaatgtggcatgttgattttttaatgtgtttatctaactgtagtgacattttctacaacgtgttttatccattccataaataataaccacattgggaatagtgaggtgtctttcattttctctgctttgtgttatttcttttcctctcaatcttttagaagtttatgtttcatgttctttggtatttattttaatatttaggataagagtcatttctaattctaatgccttttatcatatagttaatcttaatgaatcagttctactgcttggcaattttgtcatttttgtctaatttcttgttactactgctctttaattttgcttctaccatggttttcagaattttaatagtatttatatgcatttttaaaaacatttccaaacgttagaaataccatcaacatgatcagtatttttgccttttcatgtgattaatttgactatattaaattttttctcctaacatttgagataatgctccagtatccctataacataagccatttggtaccaaactttatataatatggatcacttagtagaattgttttgattaaaagaagggcttacctctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaaaaaaaaaaaaaaaa

SEQ ID NO: 5 - DNA, 1328 bp, Sus scrofa
  source 1..1328: mol_type = other DNA
gtgtgaaggtgctttcaaactttacggttttgttcctctcacccggcttttgaagagcggagaaaaagatggagaccgagtcggggtcgcaaacatcaaatcatacgcaaacagattccttgtctccatcccccaaccctgtgtcacctgtgcctttgaataatccaacgagtgccccaagatatggaacagtgatccctaatcgcatctttgtaggaggaattgactttaagacaaatgaaaatgatttaagaaaatttttttcccagtatgggtctgtaaaagaagtgaagattgtaaatgacagagctggagtgtccaaagggtatggtttcgtcacttttgaaacccaagaagatgcacaaagaattttacaagaggctgaaaaacttaattacaaggataagaaactgaacattggtccagcaataagaaaacaacaagtagggatccctcgttccagtataatgccagcagctggaacaatgtatctaacaacttcaactgggtatccttacacttatcataatggtgttgcttactttcattctccagaagtaacttctgtcccaccatcgtggccttcacgttctatatctagttcccctgtgatggtagctcagcccgtttatcagcaacctgcatatcactaccaggcacctgcacagtgtctaccaggtcagtggcagtggggtgttcctcagtcccctgcctcttctgcttcattcttatacctgcaaccttctgaggttatctatcaaccagtggaaattgcacaagatggtggatgtgttcctcctccactatctctaatggaagcctcagttccagagccttattctgatcatggagttcaagcaacatatcaccaggtttatgcttcaagtgccattgctatgcctgcacctgtgatgcagcctgaaccaattaaagtgatttgatggcactcaatagaaactggtgccccactaagaaacagaaactccaagacaattctgtagttttctaaaactatacaaccttgtagaaatactttactacacaagttttactatatgggaaattatttacttttgtttatagtgcattttgaattaatatgtaaatgttattaaatcactagaagatgagcttttcctaaaatttattaaaatagactactattaaaatattgacatttgaaacatcagcctttattcttactatgcaaattttccctggtgtaccagaaatcttgatgacttaaaatatagaaaggtagaggacttttcttgtgtcacagtaagttaaggatccagtgttgtcgctgtagtagcttgggtcactgt

SEQ ID NO: 6 - DNA, 3705 bp, Sus scrofa
  source 1..3705: mol_type = other DNA
gcgtccgcggcttctccggagcccccgagtttggcgcgtcaggcccagggggtcagactttgactcggcaaactggcctcctcgcccctcaccagccttcttccgaaatcgcccctcagccggcttctcgcactttagtaaagcctgaggtcacctccgcctctctcaccagtcccagacgccaactttcctccaaaggagaggagagggaaaggcgttcggtgcatggagctttgcataaccgttcgctttctcctccccagccgtccgaaagcaggcaagcgtggctccagaaccgagctctcggtccgcacaccccactcccgtcccgacgtgccgcacgctctgcagctgagacggcccagctcagcgagcaccagccacagccgaaactcacccgcccgcacccctccccggcttactgcgcccgcccgcaccgctgtccgccgcccaaggcgaggtcgcctgcctcccgcccccaccccccgcccgaccccaaccacccccgcccccccaccccgggtggcaggttcaggccctgggtccgagcctgagtcgctgcagctgccaccgccgccgtcgccgccagggggagagagtctccgtcgggaaacggggggctgcttctgggtgtgaaggtgctttcaaactttacggttttgttcctctcacccggcttttgaagagcggagaaaaagatggagaccgagtcggggtcgcaaacatcaaatcatacgcaaacagattccttgtctccatcccccaaccctgtgtcacctgtgcctttgaataatccaacgagtgccccaagatatggaacagtgatccctaatcgcatctttgtaggaggaattgactttaagacaaatgaaaatgatttaagaaaatttttttcccagtatgggtctgtaaaagaagtgaagattgtaaatgacagagctggagtgtccaaagggtatggtttcgtcacttttgaaacccaagaagatgcacaaagaattttacaagaggctgaaaaacttaattacaaggataagaaactgaacattggtccagcaataagaaaacaacaagtagggatccctcgttccagtataatgccagcagctggaacaatgtatctaacaacttcaactgggtatccttacacttatcataatggtgttgcttactttcattctccagaagtaacttctgtcccaccatcgtggccttcacgttctatatctagttcccctgtgatggtagctcagcccgtttatcagcaacctgcatatcactaccaggcacctgcacagtgtctaccaggtcagtggcagtggggtgttcctcagtcccctgcctcttctgcttcattcttatacctgcaaccttctgaggttatctatcaaccagtggaaattgcacaagatggtggatgtgttcctcctccactatctctaatggaagcctcagttccagagccttattctgatcatggagttcaagcaacatatcaccaggtttatgcttcaagtgccattgctatgcctgcacctgtgatgcagcctgaaccaattaaaacagtgtggagcgttcactattaagacaattgggcagctctagtccaactcaactgatttcttgtccaatgatccttgtgtggccgagatccagcttcaacgaaccggctagaagctgcccgttgtgaaatttagggttagttaatacctcaccatacttcattattccacaataagctgtctaaatttgatgcagtttgctttttcagctgttccacaaaattatcaggtagatgtggcatgttggtttttttaatgtgtattctaactgtagtgacattttctacaacatgttttatccattccataaataataatcacattgggaatagtgaggtgtccttcattttctctgctttgtattatttattttcctctcagtcttctggaagttatgtttcatattctttgcttttattttaatatttaagatgagagtcatttctaattctagtagaatttttaatactttcataccttttagcatatattatatttaatcttaatgattctgttttacctctttgctattttgtcatttttgtccatttttctgttactgctacctcttattcaactttgcttctaccatggttttcagaatttaaatagtatttatacttctttttttatagcattttccaaacatcagaaataccatgaacatgatcagtattctttctttttcatatgattaatttgacgataataaatttttccctaatatttgaaataatgctctagccttcctataacataagccatttggtaccaaattttatataatatggatcacttcatggtattgttttgataaaagaagggatcacctctgacatgatcccatagtattgacagacatttaattgtattttagtattctttaaatgcttttttcttttttttgtctttttgtcttttctagggccacacccctggcatatggaggttcccaggctaggggtccaagcagagctgtagccaccggcctacgccacagccaagtctgcgacctgtgccacagctcacagcaacgccggatcctcaacccactgagcaaggccagggattgaacccgcagcctcatggttcccagtcggattcgttaaccactgagccacgacgggaactcctgaatgctttaatcttttgaccttttctccttatgttttaccagtatattcaattatatatgtcatcagttcttaaaaggacagttatttccataaaaaacaaaactctttcttataacaaaggaacactttaaattctgattctaaagccaatcttattataaaattggaaaataagtggggaggaaaagctgttgttcagaattttatttgtaatatgaaaaaaaatccttcttcaatctttttgcaattctgtcactgaccacctaattttaggaggtaaggggaaaagtcataaatattgacattaccctaataaatctctttcctctattattccatgcccaaatatattccatgctcaatagcaacagaaataataagagttggaccccagagtttttcctgttaagtcaaatagagtgtttcttttgaaaccatggtgaatatgtttgccagtcaaatggtttcatgagaatagtagggccatataaatatatactagcatacaaggatcccatgtacattaattattctgcttttagaaatgtaaaaagactaaagtccaacaatgaaatttcaaaatttctgtaagtaaaaattattaaccactggtattagagatcttcttttaatagtactatccttgagaaagaaaaaagttaaatgttttgattttcaaaatattaaacgtgatgctaaacaaattctgaactgttaataaaaattaatgatgtattattggatgaggaattttgctaaattgtagacatccaagtgtgtttttccacagaactatatagattatatgcatgcacacatataatatatatacataatatgcatactgacatatacacattttacatatatacaggcatataaaattcatacaaatagaccttcttacaatttagccttcttttgagaagctaatcgaacctgtggaatttattgcagcctaaatatatggaggacatcctcaacctttttcatggcctagcttgata

SEQ ID NO: 7 - DNA, 4012 bp, Ovis aries
  source 1..4012: mol_type = other DNA
gccaccgccgccgtcgccgccagggggagaaagtctccgtcgggaagcgtggtgttgcttcccgggtgtgaaggtgctttcgaactgtaggtttctgcgcctttccccctggcttttgaagagcagagaggaagatggagaccgagtccggggcgcaaacatcaaatcagacgcaaacagattcattgtctccatctcctaatcctgtgtcacctgtgcctttgaataacccaacgagtgccccaagatatggaacagtgatccctaatcgaatctttgtaggaggaattgactttaagacaaatgaaaatgatttaagaaaatttttttcccagtatgggtctgtaaaagaagtgaagattgtaaatgacagagctggagtttccaaagggtatggctttgtcacttttgaaacacaagaagatgcacaaaaaattttacaagaggctgaaaaacttaattacaaggataagaaactgaacattggcccggcaataagaaagcaacaagtagggatccctcgttccagtataatgccagcagctggaacaatgtatctaacaacttcaactggatatccttacacttatcataatggtgttgcttattttcatactccagaggtaacttctgtcccaccaccttggccttcacgttctatatctagttcccctgtgatggtagctcagcctgtttatcaacaacctgcatatcactaccaggcccctgcacagtgtctaccaggtcaatggcagtggaatgttcctcagtctcctgcctcttctgctccattcttgtacctacaaccttctgaggtcatttatcaaccagtggaaattgcacaagatggtggatgtgttcctcctccaatgtctctgatggaagcttcagttccagagccttattctgatcatggagttcaagcaacatatcaccaggtttatgcttcaagtgccattgctatgcctgcaccagtgatgcaacctgaaccaattaaaacagtgtggagcattcattattaagacaattgggcagctctagtccagctcaactgatttcttatccagtgatccttgtgtggccgagatccagcttcaatgaaccggctagaagctgcccgttgtgaaacttagggttagttaatacctcaccatacttatttattccactataagctgtctaaatttgatgaaatttgctttttcagctgttctacaaaattatttaggtaaatgtggcatgttgattttttaatgtgtttatctaactgtagtgacattttctacaacatgttttatccattccataaataataaccacattgggaatagtgaggtgtctttcattttctctgctttgtgttatttcttttcctctcaatcttttagaagttatgtttcatattctgtggtatttattttaatatttaggataagggtcatttctaattctaatgccttttatcatatatttaatcttaatgaaacagttctactactttacaattttgtcatttttgtctaattttttgttaccactgctctttaattttgcttctaccatggttttcagaattttaatagtatttatacgcatttttaaaaacatttccaaatgttagaaataccatcaacatgatcagtattcttgccttttcatgtgattaatttgactatattaaattttttctcttaacctttgagataatgctccagtctccctataacataagctgtttggtaccaaactttatataatatggatcacttagtagaattgttttgatgaaaagaagggcttacctctgacatgatcttattacattgatattcttctgttttggtgctcttttaatgctttaatcctttgactttttcttatattttaccaatattttcaattatatatgtgttcttaaaaggacaattatttccataaaaagcaaatctatttcctactccaaagaaaaacattaaattataattttaaagccaattcttattatataattggaaaataggtgaggagaaaaagctgttattcaaaagttatagaataaatcgtgcttttatcactaaaatttatttttagtatgaaaaaaaccaccttcaatccatttgtaattctttcactgacctcctaattttagacaagaaggtaaagccataaatattggcattacccaaataaatctcttttcctttcttattccatctacaaatatattccatgctcagcagcagaaataataaggaccttaaagtttctcctggtgagtcaaatagggagtttcttttgaaatcataattaatatgcttgccagtcaaatggtttcatgaagacagcatgaccatatgaaatatatactagcatatgagaattccatgtacattaatttctgtcaggttttagaaatgtaaaaagacttaagtcccacaatgaaatttcaaagtttttcattttgtatgtaaaaagtactaactactggtgttagaggtcttcttttaatagtactgtccttgagaacaaaaaaagttaatgttttgactttcaaaatgttaaacatgatgctaaacaaatcctggattgttaataaaaattaatgatgcattattggataaggaatttctataattctgcatacccagatgtgttttttcagagctatgtaggttatatgtatgcacacacatatatatgtaataagcatacacacatacattttctatatatatacatgcatataattcatacatatagattttcttaaaatttagcttcattttgagaagctaatcagaactttggaaattactgaaacctaaatatatggaggatatcatcaaccttcttcatggcctagcttgatagcccaaatgtgtttttctaaaaacagacagattggaaggatgaaaattctccctacatatactaagtaaagctcatcttatttttttcctttctgtaggtttttttttggtcgtgctatgcagcttgcaggatcttatttccccgaccataggtcaaacccaggtcacaacagtgaaagcaccgaatcctaattccttggcagcattgcctatagtatatcttctatagataaatttgaagtttttacttacttagtacccattgtgaaagatcagccttaagagtgaatttagatttttattttagagtgaaatggataatatgtatatcagtcccggtaccagaatcacaaggtaataactttacaacctattttctcaaaatctctaatacaatcagagatagaaagatggtagcatggcaaaagataaagcatcttgggaaattaagacttttaacttcctatcaactgattgcacgatagcttagaaaaagacatatgatttagtttataacattgttttataaatttctgccaataatggcagtacattattttcaaacacatttatagatgataactaccatatcttaggagtgtttcctggatttatgctatgtttaactacaaaataatgaatttggatcacactaaaattattcatatttttcccagaaatagtgcttcgataatattaaactgtagaatcaaaggactagaaaaaatcttcagaaagcatccagttcaactttgatatcatgcctcaacatgctttattgtcataaatagtaaattgctcgactgtggacctatacttcgactaaaatgtactctgaaatccctgtagactttagggggaagggggcaaattatacccaaagaatggatgtaatattcataattggtactatttctttcaactgtattcagtgttttcattcctgtacataaactcctaaaaatacacttggctgtcggcttctgtacttgcaggttgagtactactctctggttaacagctaaagttattgctttcattggacctgctatgtatgtttctgtctctgcttttatgtattgcataaatttgctaattactttctgcattttcctgctttaatcaaataccattcaagatca

SEQ ID NO: 8 - DNA, 2190 bp, Bos taurus
  source 1..2190: mol_type = other DNA
atgggagatgaagattgggaagcagaaataatcaaacctcatatatcttcctatgttcctgtatttgagaaggataggtattcttctggagcaaatggagacacttttaacaggactccggcttcatcgtcagaaatgggtgatggatcatctcgaagagatcatttcatgagaagtggatttgcctctgggaggagtttgggaaacagagatcctggtgagtctaataaaagagagaatacgtccacagtgggtggttttggagttggaaagagttttggaaacagaggtttttcaaataacaagtttgaagaaggtgatagctctggtttctggagagaatctagtattgactgtgaagataatcaaacacggaacagagggttttccaagagaggcggttatcaagacggaaatgattcagaagctttaggatcgtccagaagaggtggaagaggtagtttccgaggttgccgtggaggatttggtcgaggaagtccaaatagtgattatgaacaagatgaggggacccagcgctctggtggcatttttggttctagaagatcagcattaagtggtgcaggtaatggtgacactttccaaagcagaagtggtggtggaagtggacgaggtggttacaaaggtttaaatgaagaagtaattacaggctctggaaagaattcttggaagtcagaagctgaaggaggagaaagtggtgacactcaaggaccaaaagtgacctacataccacctcctccacctgaagatgaggactccatctttgcacattatcaaacaggcataaactttgacaaatacgatactattcttgtagaagtatctggacatgatccaccaccagcaattctgacttttgaagaagctaatctttgtcagacactgaataacaacattgctaaagctggttatactaagcttactcctgtgcaaaaatacagtattcctattatacaaggaggacgagatttgatggcttgtgctcagacgggctctggaaagactgcagcttttctcttgccaattttggctcatatgatgcgtgatggaataactgccagtcgttttaaagaactgcaggaaccagagtgtattattgtagcaccaactcgagagttgatcaatcagatctatttggaagccagaaaattttcttttgggacttgtgtaagagcagttgtcatatatgggggaacccagttggggcattcaattcgacaaatagtacaaggctgtaatatattatgtgctactcctgggagactgatggatgtcataggcaaagaaaagattggtctcagacaagtcaaatacttagttttggatgaagctgatcgcatgctggatatgggttttggaccagagatgaaaaagttaatttcctgcccaggaatgccatcaaaggaacagcgtcagacccttatgttcagtgcaacttttccagaagaaattcaaaggttggctggggagtttttaaagtcgaattatttgtttgttgctgttggacaagtgggtggagcatgtagagatgttcagcagaccattcttcaagttggccagtactcaaaaagagaaaaacttgttgaaattctacgaaacataggtgatgaaagaactatggtctttgttgaaactaagaaaaaagcagattttattgccacttttctttgtcaagaaaaaatatcaacaacaagtattcatggtgatcgtgaacagagagaaagagagcaagctcttggagatttccgctgtggaaagtgccctgttcttgttgctacttcagtagctgcccgagggctggatattgaaaatgttcagcatgttattaattttgatcttccttctaccattgatgaatatgttcatcgaattgggcgtactggtcgttgtggaaatactggcagagctatttccttttttgatctggaatcagatagccagttagcacagcctctagtgaaagtgctatcagatgctcaacaggatgttcctgcatggttagaagaaattgcctttagtacatatggtcctggcttcagtggtaatgcaagaggaaatgtgtttgcatcagtcgatactagaaagaattaccacggcaagagctctttgaacacagcagggttttcttcaacacaagctcctaatccagtagacgatgagtcatgggattaa

SEQ ID NO: 9 - DNA, 2393 bp, Bos taurus
  source 1..2393: mol_type = other DNA
gggagagcaagccgcggagagccaccatgggagatgaagattgggaagcagaaataatcaaacctcatatatcttcctatgttcctgtatttgagaaggataggtattcttctggagcaaatggagacacttttaacaggactccggcttcatcgtcagatcctggtgagtctaataaaagagagaatacgtccacagtgggtggttttggagttggaaagagttttggaaacagaggtttttcaaataacaagtttgaagaaggtgatagctctggtttctggagagaatctagtattgactgtgaagataatcaaacacggaacagagggttttccaagagaggcggttatcaagacggaaatgattcagaagctttaggatcgtccagaagaggtggaagaggtagtttccgaggttgccgtggaggatttggtcgaggaagtccaaatagtgattatgaacaagatgaggggacccagcgctctggtggcatttttggttctagaagatcagcattaagtggtgcaggtaatggtgacactttccaaagcagaagtggtggtggaagtggacgaggtggttacaaaggtttaaatgaagaagtaattacaggctctggaaagaattcttggaagtcagaagctgaaggaggagaaagtggtgacactcaaggaccaaaagtgacctacataccacctcctccacctgaagatgaggactccatctttgcacattatcaaacaggcataaactttgacaaatacgatactattcttgtagaagtatctggacatgatccaccaccagcaattctgacttttgaagaagctaatctttgtcagacactgaataacaacattgctaaagctggttatactaagcttactcctgtgcaaaaatacagtattcctattatacaaggaggacgagatttgatggcttgtgctcagacgggctctggaaagactgcagcttttctcttgccaattttggctcatatgatgcgtgatggaataactgccagtcgttttaaagaactgcaggaaccagagtgtattattgtagcaccaactcgagagttgatcaatcagatctatttggaagccagaaaattttcttttgggacttgtgtaagagcagttgtcatatatgggggaacccagttggggcattcaattcgacaaatagtacaaggctgtaatatattatgtgctactcctgggagactgatggatgtcataggcaaagaaaagattggtctcagacaagtcaaatacttagttttggatgaagctgatcgcatgctggatatgggttttggaccagagatgaaaaagttaatttcctgcccaggaatgccatcaaaggaacagcgtcagacccttatgttcagtgcaacttttccagaagaaattcaaaggttggctggggagtttttaaagtcgaattatttgtttgttgctgttggacaagtgggtggagcatgtagagatgttcagcagaccattcttcaagttggccagtactcaaaaagagaaaaacttgttgaaattctacgaaacataggtgatgaaagaactatggtctttgttgaaactaagaaaaaagcagattttattgccacttttctttgtcaagaaaaaatatcaacaacaagtattcatggtgatcgtgaacagagagaaagagagcaagctcttggagatttccgctgtggaaagtgccctgttcttgttgctacttcagtagctgcccgagggctggatattgaaaatgttcagcatgttattaattttgatcttccttctaccattgatgaatatgttcatcgaattgggcgtactggtcgttgtggaaatactggcagagctatttccttttttgatctggaatcagatagccagttagcacagcctctagtgaaagtgctatcagatgctcaacaggatgttcctgcatggttagaagaaattgcctttagtacatatggtcctggcttcagtggtaatgcaagaggaaatgtgtttgcatcagtcgatactagaaagaattaccacggcaagagctctttgaacacagcagggttttcttcaacacaagctcctaatccagtagacgatgagtcatgggattaaaacgaaaacaacctgcaagtttgtggtacagttttgatgcagagaagaaagtagttttgatttttgattttttaaaacagaagtatgaaaccttgcatctcctatccttctgttcttactcccacccttaagaaaatccttactgactagttctgtgaaatgctaaaagttacaacgttgcagttactaatacaaatggtgttaaccagaaagattaaagcattctaaatgtcttgcccaaaaaaaaaaaaaaaa

SEQ ID NO: 10 - DNA, 2321 bp, Bos taurus
  source 1..2321: mol_type = other DNA
gggagagcaagccgcggagagccaccatgggagatgaagattgggaagcagaaataatcaaacctcatatatcttcctatgttcctgtatttgagaaggataggtattcttctggagcaaatggagacacttttaacaggactccggcttcatcgtcagatcctggtttttcaaataacaagtttgaagaaggtgatagctctggtttctggagagaatctagtattgactgtgaagataatcaaacacggaacagagggttttccaagagaggcggttatcaagacggaaatgattcagaagctttaggatcgtccagaagaggtggaagaggtagtttccgaggttgccgtggaggatttggtcgaggaagtccaaatagtgattatgaacaagatgaggggacccagcgctctggtggcatttttggttctagaagatcagcattaagtggtgcaggtaatggtgacactttccaaagcagaagtggtggtggaagtggacgaggtggttacaaaggtttaaatgaagaagtaattacaggctctggaaagaattcttggaagtcagaagctgaaggaggagaaagtggtgacactcaaggaccaaaagtgacctacataccacctcctccacctgaagatgaggactccatctttgcacattatcaaacaggcataaactttgacaaatacgatactattcttgtagaagtatctggacatgatccaccaccagcaattctgacttttgaagaagctaatctttgtcagacactgaataacaacattgctaaagctggttatactaagcttactcctgtgcaaaaatacagtattcctattatacaaggaggacgagatttgatggcttgtgctcagacgggctctggaaagactgcagcttttctcttgccaattttggctcatatgatgcgtgatggaataactgccagtcgttttaaagaactgcaggaaccagagtgtattattgtagcaccaactcgagagttgatcaatcagatctatttggaagccagaaaattttcttttgggacttgtgtaagagcagttgtcatatatgggggaacccagttggggcattcaattcgacaaatagtacaaggctgtaatatattatgtgctactcctgggagactgatggatgtcataggcaaagaaaagattggtctcagacaagtcaaatacttagttttggatgaagctgatcgcatgctggatatgggttttggaccagagatgaaaaagttaatttcctgcccaggaatgccatcaaaggaacagcgtcagacccttatgttcagtgcaacttttccagaagaaattcaaaggttggctggggagtttttaaagtcgaattatttgtttgttgctgttggacaagtgggtggagcatgtagagatgttcagcagaccattcttcaagttggccagtactcaaaaagagaaaaacttgttgaaattctacgaaacataggtgatgaaagaactatggtctttgttgaaactaagaaaaaagcagattttattgccacttttctttgtcaagaaaaaatatcaacaacaagtattcatggtgatcgtgaacagagagaaagagagcaagctcttggagatttccgctgtggaaagtgccctgttcttgttgctacttcagtagctgcccgagggctggatattgaaaatgttcagcatgttattaattttgatcttccttctaccattgatgaatatgttcatcgaattgggcgtactggtcgttgtggaaatactggcagagctatttccttttttgatctggaatcagatagccagttagcacagcctctagtgaaagtgctatcagatgctcaacaggatgttcctgcatggttagaagaaattgcctttagtacatatggtcctggcttcagtggtaatgcaagaggaaatgtgtttgcatcagtcgatactagaaagaattaccacggcaagagctctttgaacacagcagggttttcttcaacacaagctcctaatccagtagacgatgagtcatgggattaaaacgaaaacaacctgcaagtttgtggtacagttttgatgcagagaagaaagtagttttgatttttgattttttaaaacagaagtatgaaaccttgcatctcctatccttctgttcttactcccacccttaagaaaatccttactgactagttctgtgaaatgctaaaagttacaacgttgcagttactaatacaaatggtgttaaccagaaagattaaagcattctaaatgtcttgcccaaaaaaaaaaaaaaaa

SEQ ID NO: 11 - DNA, 2471 bp, Bos taurus
  source 1..2471: mol_type = other DNA
agagcaagccgcggagagccaccatgggagatgaagattgggaagcagaaataatcaaacctcatatatcttcctatgttcctgtatttgagaaggataggtattcttctggagcaaatggagacacttttaacaggactccggcttcatcgtcagaaatgggtgatggatcatctcgaagagatcatttcatgagaagtggatttgcctctgggaggagtttgggaaacagagatcctggtgagtctaataaaagagagaatacgtccacagtgggtggttttggagttggaaagagttttggaaacagaggtttttcaaataacaagtttgaagaaggtgatagctctggtttctggagagaatctagtattgactgtgaagataatcaaacacggaacagagggttttccaagagaggcggttatcaagacggaaatgattcagaagctttaggatcgtccagaagaggtggaagaggtagtttccgaggttgccgtggaggatttggtcgaggaagtccaaatagtgattatgaacaagatgaggggacccagcgctctggtggcatttttggttctagaagatcagcattaagtggtgcaggtaatggtgacactttccaaagcagaagtggtggtggaagtggacgaggtggttacaaaggtttaaatgaagaagtaattacaggctctggaaagaattcttggaagtcagaagctgaaggaggagaaagtggtgacactcaaggaccaaaagtgacctacataccacctcctccacctgaagatgaggactccatctttgcacattatcaaacaggcataaactttgacaaatacgatactattcttgtagaagtatctggacatgatccaccaccagcaattctgacttttgaagaagctaatctttgtcagacactgaataacaacattgctaaagctggttatactaagcttactcctgtgcaaaaatacagtattcctattatacaaggaggacgagatttgatggcttgtgctcagacgggctctggaaagactgcagcttttctcttgccaattttggctcatatgatgcgtgatggaataactgccagtcgttttaaagagctgcaggaaccagagtgtattattgtagcaccaactcgagagttgatcaatcagatctatttggaagccagaaaattttcttttgggacttgtgtaagagcagttgtcatatatgggggaacccagttggggcattcaattcgacaaatagtacaaggctgtaatatattatgtgctactcctgggagactgatggatgtcataggcaaagaaaagattggtctcagacaagtcaaatacttagttttggatgaagctgatcgcatgctggatatgggttttggaccagagatgaaaaagttaatttcctgcccaggaatgccatcaaaggaacagcgtcagacccttatgttcagtgcaacttttccagaagaaattcaaaggttggctggggagtttttaaagtcgaattatttgtttgttgctgttggacaagtgggtggagcatgtagagacgttcagcagaccattcttcaagttggccagtactcaaaaagagaaaaacttgttgaaattctacgaaacataggtgatgaaagaactatggtctttgttgaaactaagaaaaaagcagattttattgccacttttctttgtcaagaaaaaatatcaacaacaagtattcatggtgatcgtgaacagagagaaagagagcaagctcttggagatttccgctgtggaaagtgccctgttcttgttgctacttcagtagctgcccgagggctggatattgaaaatgttcagcatgttattaattttgatcttccttctaccattgatgaatatgttcatcgaattgggcgtactggtcgttgtggaaatactggcagagctatttccttttttgatctggaatcagatagccagttagcacagcctctagtgaaagtgctatcagatgctcaacaggatgttcctgcatggttagaagaaattgcctttagtacatatggtcctggcttcagtggtaatgcaagaggaaatgtgtttgcatcagtcgatactagaaagaattaccccggcaagagctctttgaacacagcagggttttcttcaacacaagctcctaatccagtagacgatgagtcatgggattaaaacgaaaacaacctgcaagtttgtggtaccgttttgatgcagagaagaaagtagttttgatttttgattttttaaaacagaagtatgaaaccttgcatctcctatccttctgttcttactcccacccttaagaaaatccttactgactagttctgtgaaatgctaaaagttacaacgttgcggttactaatacaaatggtgttaaccagaaagattaaagcattctaaatgtaaaaaaaaaaaaaaaaaaaaaaaaaa

SEQ ID NO: 12 - DNA, 2996 bp, Bos taurus
  source 1..2996: mol_type = other DNA
ggcacgaggtctgctgccgctccctctgcccagtccggccgggcagcgggcacgagcagacccaacagcagctccaagcgcggtgcccgaggccaggctctgcttcgtcctcgagcccctctgagtgaacactggctgtcgccatcatgtctgctgcaaatcctgagactccaaactcaaccatctccagagaggccagcacccagtcgtcttcagctaccaccagccaaggctatgttttaccagaaggcaaaatcatgccaaacactgtttttgtcggtggaattgatgttaggatggatgaaacagaaattagaagtttctttgctagatatggttcagtaaaagaagtgaagataatcacggatcgaactggtgtgtccaaaggctatggatttgtttcattttataatgacgtggatgtgcagaagatagtagaatcacagataaatttccatggtaaaaagctgaaactgggccctgcaatcaggaaacaaaatttgtgtgcttatcatgtgcagccacgtcctttggtttttaatcctcctcctccaccacaatttcagagtgtctggagtaatccaaacgctgaaacttacatgcagcctccaaccatgataaaccctataactcagtatgttcaggcttatcctccttatccaagttcaccagttcaggttatcactggatatcagctgcctgtttataattatcagatgccaccgcagtggcctgctggggaacaaaggagttatgttattcctccggcttatacaaccattaactaccactgtaatgaagttgacaccggagctgacgttttgcccagtgaatgctcggttcatgaagctactccgtcctctggaaatggcccacaaaagaaatctgtggacagaagcatacagacggtggtatcttgtctatttaatccagagaatagactgagaaactctgttgtcactcaagatgactacttcaaggataaaagagttcatcactttagaagaagtcgggcagtgcttaagtctgtttgatcctctctgctaactttctagtctcataggaagtcacaggttttgaatagtaagagactgaaagttttccactgttatagaaatttaattctgaattttgataaatcacactcagactttctatatgttatattagattgcttagttttattttacattgaaactttttcattagacatttacttagaaactggttctgtgttgaatatatagttaaaagttaaaaaataattgagactgaaaagaataactttaagatatatctgtaaggatttttacctgacattttaagagtttaaaaacaaatgctgattttttgggaaaaaaaaattgttttagaaaacctattttgaaaggtcagaattttgatagtttaaatacaagcacttcacttctccaacaagtacctatgaacagtacagtatctgcaatattgtgcttttatgatttgtctagaaatttaccacaaaagcagagtttttaaaactgaatttttaatcagtgaaactcaatgtatagttagctttattgaagttttccttatctaaacccagtaaaacagattctaaacaaacagtccaatcagtgggtcttatgtttattacttcaaaatactttatcttttatctagaatccacacatagaggtatctgatgggatggtaattaggattactaaaattctgggccaaatttttttttaaaagaatctaaaacaaacttaacttttctaggtacataaacttcttagttgagttaccattcttttttcttttactttttttttccccttgaaatgttaaactcaatggctgtattttaaattgtgcaaaatattggtattaaagaaggctgaaactttgtcacttaaaagttaatcagagtatctgaaaggaattgtttttataagaaacattaaaatattagctatttgttaaagaacagatagattttaatttttttttagcctgttatttttctttcttttttttttttttaagtaaaatatgtccaggagagtcagagtagttttgatgtttctaaaccacaaaagttgtttagtaataagcatatccccaaaacacacttgagttagttaacagtccatttttagatgaggtggctcctcctcagacattgtaaatcgtgacaagcacctggagatccagagcccctttgctgtacccacagtcttgcttctccagtctccactgttcctgcaggtgctcctggaacagcgatggaaaggaaaggaggaggagtataagcagtttgagggttaagccttggaaggaaagggtagcagtgggataatccctttctaagggattcacttggagcagtatcgtttgacctgccatgggtttagaggagcttcctggcttttctgtcttgggccagccccttcattaaaacacggaaactccgtatagccctgtcctgacgaggcttccgtgggccgtgccatcagtgcccgttgtcttagcactcctaataattttcgttctgaatctgaagggaagaatcttttaacttaatgatacccaagagggcttttttatgcctcagaatttgaaagtgccctgaaatttgttctttttaaaattctctgtacttttttgtgtgtgtgctataacattcttacatgtattattctgtgattaaatctccaggttactaaaaatgatacctaaattctaaagagccaattctcattattccagtatgaccttattcccttaagaaaaagggaataaacattttgtctgctattttgttggactgaagtatttaaaagagtaaggtagaagagacaaaatcctgaacctttcaaaatggaaaattaattttaaacttaaaaatgtgttcctattacctatgctgatactgtctttgcataaatgaataaatagaaataaagaaaaaaaaaaaaaaaaaaaaaaaaaaaaaaaaaaaaaaaaaa

SEQ ID NO: 13 - DNA, 2456 bp, Bos taurus
  source 1..2456: mol_type = other DNA
tacccgcctctgactctctccgtcgggtctgctgccgctccctctgcccagtccggccgggcagcgggcacgagcagacccaacagcagctccaagcgcggtgcccgaggccaggctctgcttcgtcctcgagcccctctgagtgaacactggctgtcgccatcatgtctgctgcaaatcctgagactccaaactcaaccatctccagagaggccagcacccagtcgtcttcagctaccaccagccaaggctatgttttaccagaaggcaaaatcatgccaaacactgtttttgtcggtggaattgatgttaggatggatgaaacagaaattagaagtttctttgctagatatggttcagtaaaagaagtgaagataatcacggatcgaactggtgtgtccaaaggctatggatttgtttcattttataatgacgtggatgtgcagaagatagtagaatcacagataaatttccatggtaaaaagctgaaactgggccctgcaatcaggaaacaaaatttgtgtgcttatcatgtgcagccacgtcctttggtttttaatcctcctcctccaccacaatttcagagtgtctggagtaatccaaacgctgaaacttacatgcagcctccaaccatgataaaccctataactcagtatgttcaggcttatcctccttatccaagttcaccagttcaggttatcactggatatcagctgcctgtttataattatcagatgccaccgcagtggcctgctggggaacaaaggagttatgttattcctccggcttatacaaccattaactaccactgtaatgaagttgacaccggagctgacgttttgcccagtgaatgctcggttcatgaagctactccgtcctctggaaatggcccacaaaagaaatctgtggacagaagcatacagacggtggtatcttgtctatttaatccagagaatagactgagaaactccgttgtcactcaagatgactacttcaaggataaaagagttcatcactttagaagaagtcgggcagtgcttaagtctgtttgatcctctctgctaactttctagtctcataggaagtcacaggttttgaatagtaagagactgaaagttttccactgttatagaaatttaattctgaattttgataaatcacactcagactttctatatgttatattagattgcttagttttattttacattgaaactttttcattagacatttacttagaaactggttctgtgttgaatatatagttaaaagttaaaaaataattgagactgaaaagaataactttaagatatatctgtaaggatttttacctgacattttaagagtttaaaaacaaatgctgattttttgggaaaaaaaattgttttagaaaacctattttgaaaggtcagaattttgatagtttaaatacaagcacttcacttctccaacaagtacctatgaacagtacagtatctgcaatattgtgcttttatgatttgtctagaaatttaccacaaaagcagagtttttaaaactgaatttttaatcagtgaaactcaatgtatagttagctttattgaagttttccttatctaaacccagtaaaacagattctaaacaaacagtccaatcagtgggtcttatgtttattacttcaaaatactttatcttttatctagaatccacacatagaggtatctgatgggatggtaattaggattactaaaattctgggccaaatttttttttaaaagaatctaaaacaaacttaacttttctaggtacataaacttcttagttgagttaccattcttttttcttttacttttttttcccccttgaaatgttaaactcaatggctgtattttaaattgtgcaaaatattggtattaaagaaggctgaaactttgtcacttaaaagttaatcagagtatctgaaaggaattgtttttataagaaacattaaaatattagctatttgttaaagaacagatagattttaatttttttttagcctgttatttttctttctttttttttttttttaagtaaaatatgtccaggagagtcagagtagttttgatgtttctaaaccacaaaagttgtttagtaataagcatatccccaaaacacacttgagttagttaacagtccatttttagatgaggtggctcctcctcagacattgtaaatcgtgacaagcacctggagatccagagcccctttgctgtacccacagtcttgcttctccagtctccactgttcctgcaggtgctcctggaacagcgatggaaaggaaaggaggaggagtataagcagtttgagggttaagccttggaaggaaagggtagcagtgggataatccctttctaagggattcacttggagcagtatcgtttgacctgccatgggtttagaggagcttcctggcttttctgtcttgggaagactgg

SEQ ID NO: 14 - DNA, 1373 bp, Bos taurus
  source 1..1373: mol_type = other DNA
ggcacgaggtctgctgccgctccctctgcccagtccggccgggcagcgggcacgagcagacccaacagcagctccaagcgcggtgcccgaggccaggctctgcttcgtcctcgagcccctctgagtgaacactggctgtcgccatcatgtctgctgcaaatcctgagactccaaactcaaccatctccagagaggccagcacccagtcgtcttcagctaccaccagccaaggctatgttttaccagaaggcaaaatcatgccaaacactgtttttgtcggtggaattgatgttaggatggatgaaacagaaattagaagtttctttgctagatatggttcagtaaaagaagtgaagataatcacggatcgaactggtgtgtccaaaggctatggatttgtttcattttataatgacgtggatgtgcagaagatagtagaatcacagataaatttccatggtaaaaagctgaaactgggccctgcaatcaggaaacaaaatttgtgtgcttatcatgtgcagccacgtcctttggtttttaatcctcctcctccaccacaatttcagagtgtctggagtaatccaaacgctgaaacttacatgcagcctccaaccatgataaaccctataactcagtatgttcaggcttatcctccttatccaagttcaccagttcaggttatcactggatatcagctgcctgtttataattatcagatgccaccgcagtggcctgctggggaacaaaggagttatgttattcctccggcttatacaaccattaactaccactgtaatgaagttgacaccggagctgacgttttgcccagtgaatgctcggttcatgaagctactccgtcctctggaaatggcccacaaaagaaatctgtggacagaagcatacagacggtggtatcttgtctatttaatccagagaatagactgagaaactctgttgtcactcaagatgactacttcaaggataaaagagttcatcactttagaagaagtcgggcagtgcttaagtctgtttgatcctctctgctaactttctagtctcataggaagtcacaggttttgaatagttactaaaaatgatacctaaattctaaagagccaattctcattattccagtatgaccttattcccttaagaaaaagggaataaacattttgtctgctattttgttggactgaagtatttaaaagagtaaggtagaagagacaaaatcctgaacctttcaaaatggaaaattaattttaaacttaaaaatgtgttcctattacctatgctgatactgtctttgcataaatgaataaatagaaataaagaaaaaaaaaaaaaaaaaaaaaaaaaaaaaaaaaaaaaaaaaa

SEQ ID NO: 15 - DNA, 2996 bp, Bos taurus; identical to SEQ ID NO: 12
  source 1..2996: mol_type = other DNA

SEQ ID NO: 16 - DNA, 2235 bp, Sus scrofa
  source 1..2235: mol_type = other DNA
aaccggaagccaccatgggagatgaagattgggaagcagaaataatcaaacctcatatgtcttcctatgttcccgtatttgagaaggataggtattcttctggagcaaatggagacacttataataggactccagcttcatcatcagaaatggatgatggatcttctcgaagagatcacttcatgagaagtggctttgcctctgggaggagtttgggaaaccgggatcctggcgaatctaataaaagagagaatacatccacaatgggtggttttggtgttggaaagagttttggaaacagaggtttttcaaataacaagtttgaagaaggtgatagctctggtttctggagagagtctaataatgactgtgaagataatcagacacggaacagaggtttttccaagagaggcggctttcaagatggaaatgattcagaagcctcagtgtcgtccagaagaggtggaagaggtagtttccgaggttgccgtggacgatttggtctaggaagtccaaatagcgaatatgaacaagatgaggggacccatcgtactggtggcttttttggttctagaagatcagcattaagtggtgcaggtaatggtgacacttaccaaagcagaagtggtagtggaagtggacgaggtggttacaaaggtctaaatgaagaagtaattacaggctctggaaagaattcttggaagtcagaagctgaaggaggagaaagtggtgacattcaaggaccaaaagtaacctacataccacctcctccccctgaggatgaggactccatctttgcacattatcagacaggcataaactttgacaaatatgatactattcttgtagaagtatctggacatgaggcaccgccagcaattctgacttttgaagaagctaatctctgtcagacactgaatagcaacattgctaaagctggttatactaagcttactcctgtacagaagtacagtattcctattatactaggaggacgagatttgatggcttgcgctcagacagggtctggaaagactgcagcctttctcttgccaattttggctcatatgatgcgtgatggaataactgccagtcgttttaaagagctgcaggaaccagagtgtattattgtagcaccaactcgagaattgatcaatcagatctatttggaagccagaaaattttcttttgggacttgtgtaagagctgttgttatatatgggggaacccagttggggcattcaattcgacaaatagtacaaggctgtaatatattatgtgctactcctgggagactgatggatatcataggtaaagaaaagattggtctcagacaagtcaaatatttagttttggatgaagctgatcgcatgctggatatgggttttggaccagaaatgaagaagttaatttcctgcccaggaatgccatcaaaggaacaacgtcaaacacttatgtttagtgcaacttttccagaagaaattcaaaggttggctggggagtttttaaaatcgaattatttgtttgttgctgttggacaagtgggcggagcatgtagagatgttcagcagaccattctccaagttggccagtactcaaaaagagaaaaacttgttgaaattctacgaaacataggtgatgaaagaactatggtctttgtagaaactaagaaaaaagcagattttattgccacttttctttgtcaagaaaaaatatcaacaaccagtattcatggtgatcgagaacagagagagagagagcaagctcttggagattttcgctgtgggaagtgtccagttcttgttgctacttcagtagctgcccgagggctggatattgaaaatgttcagcatgttatcaattttgatcttccttccaccatcgatgaatatgttcatcgaattgggcgtacgggtcgctgtggaaatactggcagagctatttccttttttgatcctgaatcagatagccatttagcacagccactagtgaaagtactatcagatgctcaacaggatgttcctgcgtggttagaagaaattgcatttagtacctatgttcctggcttcagtggtagttcaagaggaaatgtgtttgcatcagttgatactagaaagaattaccagggcaagagctctttgaacacagcagggttttcttcttcacaagctcccaatccagtagatgatgagtcatgggattaaagctaaaacaaccttcaagtttgtggcagtt

SEQ ID NO: 17 - DNA, 219 bp, Ovis aries
  source 1..219: mol_type = other DNA
ttcagaagcttcaggatygtccagaagaggtggaagaggtagtttccgaggttgccgtggaggatttggtttaggaagtccaaatagcaattatgaacaagatgaggggacccagcgctctggtggcatttttggttctagaagatcagcattaagtggtgcagataatggtgacactttccaaagcagaagtggtagtggaagtggacgaggtggtta

SEQ ID NO: 18 - DNA, 894 bp, Sus scrofa
  source 1..894: mol_type = other DNA
atgtctgctgcaaatcctgagactccaaactcaactatctccagagaggccagcacccagtcttcatcagctgcaaccagccaaggctatgttttaccagaaggcaaaatcatgccaaacaccatttttgttggtggaattgatgttaggatggatgaaaccgaaattagaagtttctttgctagatatggttcagtaaaagaagtgaagataatcacagatcgaactggtgtgtccaaaggctatggatttgtttcattttataatgacgtggatgtgcagaagatagtagaatcacaaataaatttccatggtaaaaagctgaaactgggccctgcaatcaggaaacaaaatttgtgtgcctatcatgtgcagccacgtcctttggtttttaatcctcctcctccaccacagtttcagagtgtctggagtaatccaaacactgaagcttacatgcagcctccaaccatgatgaatcctataactcagtatgttcaggcttatcctccttacccaagttcaccagttcaggttatcactggttatcagctgcctgtgtacaattatcagatgccgccacagtggcctgctggggaacaaaggagttatgttattcctccggcttatacagctgttaactaccactgtaatgaagttgatccaggagctgaagttttgcagagtgaatgctcagttcatgaagctgctccatcctctggaaatggcccacaaaagaaatctgtggaccgaagcatacagacagtggtatcttgtctgtttaatccagagaacagactgagaaactcccttgttactcaagatgactacttcaaggataaaagagttcatcactttagaagaagtcgagcagtgcttaagtctgtttgatcctct

SEQ ID NO: 19 - DNA, 3899 bp, Sus scrofa
  source 1..3899: mol_type = other DNA
tatttgtgaccagttggccaccattttgccagctcacaaagtaatcctcagtacaaaagtgaagtttgaggataagttacagccagttattctctgtggatataaggttctggagtggtttccaaagttgtgtgatttgctctgcaaactgccccatagctcgcccttgctctaggccctgtttctctgatcttgagaatgttctagaattctctaggcaggcctaacttcttcctttaaggtcaggaagaaaaatccccccccttcacaatttgaacacccccagcaaaggaacgtttgggtattttattcactcctacattttgggtgcctggcaattgttgatctcaataacttgtttctggaaagaacaaacgtcttgaatgccaagcgggcctgcctacctgatgtttccaatccaaccccatctccctcacacttgaaagaaagtcatccaagtttctggacttttgatgtctccctccctgttcttcatatatgtctttctgttttctatttctgttgccatttaaattgatttgggggctgatgatagggataatcgccattgggctattcctgggcctttgggacgccattttgaaactagagcctggacacattcaaattttgtttttaattttaacacatatttgagtgtgcagacttatttcaggtgtgcttctaaggcactttcgggactttctgtatatctgtgggtccgtcggcacgcccttcgggaagtggcgcctctaaggggtatgctgtcggcccgcgcgcaccccttgcgcacccctggctggcgccgccgtgccccgcgagccccctctgaggggggcgggggcgcggccgcccattggctgaggcacctcggacgagcccccccacaccccgcgcaggagccacctccgggcgccctgctcgccgcgcctcagtccgccagcacccctcagcctcgccagctgcctgcgggtcgctttgcttatccgcttctgcttcctctgcccactcgaagccggacaacaggcaccagcaggcctacagcagccccgagcacggtgctgaagccagcctctgctcctccagtccctcggtgggaacactggctgtcgcagtaatgtctgctgcaaatcctgagactccaaactcaactatctccagagaggccagcacccagtcttcatcagctgcaaccagccaaggctatgttttaccagaaggcaaaatcatgccaaacaccatttttgttggtggaattgatgttaggatggatgaaaccgaaattagaagtttctttgctagatatggttcagtaaaagaagtgaagataatcacagatcgaactggtgtgtccaaaggctatggatttgtttcattttataatgacgtggatgtgcagaagatagtagaatcacaaataaatttccatggtaaaaagctgaaactgggccctgcaatcaggaaacaaaatttgtgtgcctatcatgtgcagccacgtcctttggtttttaatcctcctcctccaccacagtttcagagtgtctggagtaatccaaacactgaagcttacatgcagcctccaaccatgatgaatcctataactcagtatgttcaggcttatcctccttacccaagttcaccagttcaggttatcactggttatcagctgcctgtgtacaattatcagatgccgccacagtggcctgctggggaacaaaggagttatgttattcctccggcttatacagctgttaactaccactgtaatgaagttgatccaggagctgaagttttgcagagtgaatgctcagttcatgaagctgctccatcctctggaaatggcccacaaaagaaatctgtggaccgaagcatacagacagtggtatcttgtctgtttaatccagagaacagactgagaaactcccttgttactcaagatgactacttcaaggataaaagagttcatcactttagaagaagtcgagcagtgcttaagtctgtttgatcctctctcctaactttctagtcccatgggaaattgcaggttttgagtaatgagaagggactgaaagtttttccactcttatagaaatttaaatctgaattttgataaatcacactcagactttctatacaagttagattacctagttttattttacattgaagctatttttcattagacgtttacttagaaactggttctgtgttgaatatgtagttaaaagtaaaaaataattgatactgaaagaataactttaagatttatttgtaaggattttttaaatagactttttaagagtttaaaagcaaatactgattttcaaaatattgttttagagaacctgttttgaaaggtcagaattttgatagtttaaatataagcatttcatttctccaggcacctatgaacagtacagtatctacaatattgtgctttacatttatgattagttttgaaatttaccacaaaagcagagttttttaaactgcaattttaatcagtggaactcagtatatagttagctttattgatgtttctttgtctaaacccagtaaaacatattccaaacagtccaatcagtgggtcttacatttattcaaaatgttttatcttttatctagaatccacacatagagataaccgattgggatggtaattaggataactaaaattctgggcctcatttttttttaaagaatccaaaacaaactaaactttactaggtacataagcttcttggtgagtttccattctccttttttttttcttttttttttttttcatgaaatgctaaactcgatggcggtatcttaaattgtgcaaaatattcgtattgaggaaggctgaaactttttttatgtcacttgaaagttaatcagagtatctgaaaggaattgtttttctaaaaacattaaaatattagttatttgttaaagaacaggtaaaatttttacttttgttttttaacctattatatatttttttttaagtaaaatatgtccaggagagtgaaagtaattttagcatttctaaaccacaaatgtttagtaataagcgtatccccaaaatatacttgagttagagtaaacaatccatttctaattaggtaggctccttgttagatgtcgtaaatcgtgatgagcagtagaagtgccagaacccctttgctgacccacagtcttggttttctagtctgtagcaccattcttccaggaggtttgcttctagaataggtgatgtaaagaaaagtagcagtataagcagtttgagggcagtcaagccttgggcaggtagggaaacttgtatcagcatcatttgacctgttggggacatgagttcaaagtggctttctggcttttctttcattggccagtccctcactacaacatgtagactccaatttatccctatcttaatgaagttccagggaccatatcatcagtgcccattgttatctcataattcctaataattttcattctgaatctgaagggaagagtcttttaacttaaggattcccaagaaggctttcttatgccttagaatgtaaaagtgccctaaaatttgtcctttttaaaattctctatacttttttttgtgcgtgctataacattcttacatgtattattctgtgattaaatctctgggttactataaatgatacctaaattctaaagagccagttctaattatcccaatatgactttatgaaaagggagggaataaattttgtctgctgttttgttggactgatttaaaagagtaaggtagaagagacaagatcctgaacctttcagaatggagaattaattttaaacttaaaagtttgttcccattactatactgatactgtctttgaataaatgaataaataaagaaactttttttttcaaaa

SEQ ID NO: 20 - DNA, 2941 bp, Ovis aries
  source 1..2941: mol_type = other DNA
cgccacccgcctctgactctctgcccggtgtgccgccgctccctgtgcccagtccggccgggcagcgggcacgagcagacccaacagcagctccgagcgcggtgcccgaagccagcctctgtttcgtcctcgagcccctctgagggaacactggctgtcgccatcatgtctgcagcaaatcctgagactccaaactcagccgtctccagagaggccagcacccagtcttcttcagctgccaccagccaaggctatgttctgccagaaggcaaaatcatgccgaacactgttttcgttggtggaattgatgttaggatggatgaaacagaaattagaagtttctttgctagatatggttcagtaaaagaagtgaagataatcacggatcgaactggtgtgtccaaaggctatggatttgtttcattttataatgacgtggatgtgcagaagatagtagaatcacagataaatttccatggtaaaaagctgaaactgggccctgcaatcaggaaacaaaatttgtgtgcttatcatgtgcagccacgtcctttggtttttaatcctcctcctccaccacaatttcagagtgtctggagtaatccaaacactgaaacttacatgcagcctccaaccatgatgaaccctataactcagtatgttcaggcttatcctccttatccaagttcaccagttcaggttatcactggatatcagctgcctgtttataattatcagatgccaccgcagtggcctgctggggaacaaaggagttatgttattcctccggcgtatacaaccattaactaccactgtaatgaagttgatacaggagctgaagttttgcccagtgaatgttcagttcatgaagccactccatcctctggaaatggcccacaaaagaaatcggtggacagaagcatacagacggtggtatcttgtctatttaatccagagaatagactgagaaactccgttgtcactcaagacgactacttcaaggataaaagagttcatcactttagaagaagtcgggcagtgcttaagtctgtttgatcctctctgctaactttctagtctcataggaagtcacaggtgttgaatagtaagagactgaaagttttccactattatagaaatttaattctgaattttgataaatcacactcagactttctatataagttatattagattgcttagttttattttacattgaaagtttttcattagacatttacttagaaactggttctgtgttgaatatatagttaaaagtaaaaaaataattgagactgaaaagaataacttttaagatatatctgtaaggatttttacttgacattttaagagtttaaaaacaaatgttgattttttgaaaaaaaaaattgttttagaaaacctattttgaaaggtcagaattttgatagtttaaatacaagcacttcacttctccaacaagtacctgtgaacagtacaatatctgcaatattgtgcttttatgatttgtctagaaatttaccacaaaagcagagtttttaaaactgcatttttaatcagtgaaactcaatatatagttagctttattgaagttttccttatctaaacccagtaaaacagattgtaaacaaacagtccaatcagtgggtctgatgtttattacttcaaaatactttatcttttatctagaatccacacatagagatatccaattgggatggtaattaggataactaaaattctgggtcaaaaattttttttaaagaatctaaaacaaattaacttttctaggtacataaacttcttagttgagttaccattctccttttttcttttacttttttttttccttgaaatgctaaactcaatggctgtattttaaattgtgcaaaatattggtattaaagaaggctgaaacttttcgtcacttaaaagttaatcagagtatctgaaaaggaattgtttttataagaaacattaaaatattagttatttgttaaagaacagatagatttttacttttatttttttagcctgttatctttctttctttctttctttcttaagtaaaatacgtccaggagagtcagagtagttttgatgtttctaaaccacaaaagttgtctagtaataagcatatccccaaaatacacttgagttagttaacaatccatttttagattaggtggctcctcctcagacattgtaaatcgtgacaagcaactagagatccagagcccctttgctagacccacagtcttgctttcccagtctccaccgttgttgcaggtttgctcctggagcagcgacggaagggaaagaagtagtagtataagcattttgaaggttgagccttggaaagaaagagtagcagtaggataacccctttctaagggaatcacttgtagcagcatcgtgtgacctgccatggatttagaggagcttcctggcttttctttcttgggccagccccttcattaaaacgtggaaactccatatagccctgtcctaatgaagcttccatgggccattccatcagtgcccattgtcttatcattcgtagtaattttcattctgagtctgaagggaagaatcttttaacttaatgatacccaagagggctttcttacgcctcagaatttgaaagtgccctgaaatttgttctttttaaaattctctgtacttttttgtgtgtgctataacattcttacatgtattattctgtgattaaatctccaggttactataaatgatacctaaattctaaagaaccaattctcattattccagtatgaccttattcccttaagaaaaagggaataaacgttttgtctgctattttgttggactgaagtatttaaaagagtaaggtagaaagagacaaaatcctgaacctttcaaaatggaaaattaattttaaacttaaaaatgtgttcctattacc

SEQ ID NO: 21 - DNA, 2395 bp, Bos taurus
  source 1..2395: mol_type = other DNA
agcagcgcgcgctcactgcccgcccgccccccgcgcgtccgcctccgctccgcctccgtcccgggcgcgggcgaggtggcccgggcgggggagggcagggggcagggccgcggcggagaagccgcggggcggggaggagcgggtcccgataaaaggcggcgaggcagccccaccccgaggcaggccggcgggcaggctcggctcgtcccttccgtccggcccgcgccggcggcggggaggcggcgcgcgcggcccgcaacccgcccatggaggctttcccctgggcgccccgctcgccccgccgcggccgcgcccccccgcccatggcgctcgtgcccagcgcccgctacgtgagcacccagggcccggcgcacccgcagcccttcagctcgtggaacgactatctgggactcgccacgctcatcaccaaggcggtggacggcgagccgcgcttcggctgcgcccgcggcggggacggcggcggggacggctccccgccttcttcttcctcctcgtcgtgctgctccccccacgtgggggccgggcctggggcgctggggcccgcgctggggccgcccgactacgacgaggacgacgacgacgacgacagcgacgatccggggtcccggagccgctacctggggggcgcgctggagctgcgcgcgctggagctgtgcgcggaccctgccgaggccgggctgctggaggagcgtttcgctgagctgagcccgttcgctggtcgcgccgctgccgtgcttctgggctgcgcacccgccgccgccgccgcggccaccgccgaagcagcaccgcgagaggagcgggccccggcgtgggcggccgagcccaagctgcacgccgcctccggggcggccgccgcccggctgctcaagcccgagctgcaggtgtgcgtgttttgccggaacaacaaggaggcggtggcgctctacaccacccacatcctgaagggacccgacgggcgggtgctgtgccccgtgctgcgccggtacacgtgtcccctgtgcggtgccagcggcgacaacgcgcacaccatcaagtactgcccgctttccaaagtgccgccgccgcctgcagcccgcccgccgccgcgcagcgcccgggacggcctgcccggcaagaagctgcgctaagggcccggaccccggtctgctgctgccacctgatgccactggggtagccgcccgcccactctcgtgtttggtctgcgcaccatctcttcctcgctgccggggagtgtggagctcgtcttggtttttccagaggaagccgacggtaccgagtattttcctaacgaagagcagttgagactagacgttaaaattttgattaatgtttctagtttgtgcacatccagatggtgaaggctgggtattccactaactgaaatgtggcaacttagaggcgctgtggtttatttatacgtcgacctattttagatgcgcatcagtatgaaattgtctcagtctaatcttggatgtttaattttatgaatggaggcactttactaggtctagaatatttttttaaaagcctctcaactgaacttaaaactggcgattttatggagtgtcagcaaaatgactattttattgtctgaaacaatatttctgttgtccttacccagttgtaattccaggtgaagccctgcgtggtagcatcattaagtgaagacttggtatgctttacagtgttagtttgggtgggtgttccctccttgtggcttgtttttgtcctagctggagatgtataaaatgtacaatttgtaggtagcaggtagaatacagctcatgtaccagatctttttattactgaacgagcaactactaccgtttttcccctttaaaaatagtgccaagttataatcatattgtgtatacacttgaaaatggtgctgtttaaaaaaattgtgtatttatacagtaacagtatatgaattcattaaccttgcctttaactctacttggctttttctttatgccccttcctattccagttcttcaaaaatatgtgatacttaagatcaaacgggtgcaataactcattcactctgaattgctccatttcagggtctctaaatagtggaaatctcattccagctgttgcctctcagactaaatgtaagatggaatcctttgagctctggaaggttaatgaaacaactggtgttcaggaaggttccactctggactgtgtcagctttaaaccatcacagaagtcctcaaaccagtataagtaccaattaaaggaactgactgggtgtagggggggtaacacaaggaacacagcctccatctattgtgttcccattctcattagaagacaacccttctggaatcccaccagttattttcatcggtgagattaaatctaatcttgg

SEQ ID NO: 22 - DNA, 1897 bp, Sus scrofa
  source 1..1897: mol_type = other DNA
gcgtcctttccgtccggcccgcgccggcggcggggaggcggcgcgcgcggcccgcagcccgcccatggaggctttcccctgggcgccccgctcgccccgccgcggccgcgtccccccgcccatggcgctcgtgcccagcgcccgctacgtgagcgcccagggcccggcgcacccgcaacccttcagctcgtggaacgactacctgggactcgccacgctcatcaccaaggcggtggacggcgagccgcgctttggctgcgcccgcggcgaggacggcggcggcggcggcggctccccaccctcctcttcctcctcgtcgtgctgctccccccacgcgggggccgggcctggggcgctggggcccgcgctggggccacccgactacgacgaggacgacgacgacgacagcgacgagccggggtcccggggccgctacctggggggcacgctggagctgcgcgcgctggagctgtgcgcggacccctcggaggccgggctgctggaggagcgcttcgccgagctgagcccgttcgcgggtcgcgccaccgctgtgctgctgggctgcgcacccgccactgccgccaccgccgaggcggcaccgcgcgaggagcgggccccggcgtgggcggccgagccccggctgcacgcagcctctggggcggccggcgcccggctgctcaagcccgagctgcaggtgtgtgtgttttgccggaacaacaaggaggcggtggcgctctacaccacccacatcttgaagggacccgacgggcgagtgctgtgccccgtgctgcgccgctacacgtgtcccctgtgcggcgccagcggcgacaacgcgcacaccatcaagtactgcccgctctccaaagtgccgccgccgcgcagcgtcagggacggcctgcccggcaagaagctgcgctgagggcccggactccggtctgctactgccacctgacgccaccagggtcgccgcctgcccaatgtctagtttggcctgcgcaccatctctctctctcgctgctgaggagcgtggagctcagctgttggttgaacttgagatgtactgattttttttttttttttttcaaaagaacccggcggtactgagtcctttcctgtcgaagagcgcttaagactagaagctaaaatcttgatttgtttatctctagtttgtgcacatccagacggtgaaggctgggtgttcgttccactaactgaaatgtggcaacttagaagtgtttatttactctatacgtcaacctattttagatgcgcatcagtatatgaaattgtctcaatctaatcttggatgtttaattttatgaatggaggcactttactaggcctagaatatttttttaaaagcctctaaactgaacttaactggcgattttatggaatgtcagcaaaatgacttttattgtttgaaacaagtaataatatttctgttgtccttaatcagttattctaattccaggtgaagcaaccctcacctgcctggtagcatcattaagtgaaggcttagtaaactttccagtgttagtttgggtgggtgttccccccgtggcttgtttctgtcctagctggaggtgtaaagatgtacaatctgtggcaggtagaatacagctccttatccttttatgtaccacatcttttattactgaacgagcaactagcgtttcccatctttcaaagtcgtgccaggttatataatattgtgtatacacttggaaatggtgctgtttaaaagaatttgtgtatttatacagtaacagtatatgaattcattaatcttgtctttcactctggtacttggccttttgtctgtatacctccacctcgccatttctagttcaaaaatatttgattattgaagatcaaatggagccgtatgcactct

SEQ ID NO: 23 - DNA, 1792 bp, Ovis aries
  source 1..1792: mol_type = other DNA
gaggcaggccggcgggcaggctcggctcgtcccttccgtccggcccgcgccggcggcggggaggcggcgcgcgcggcccgcaccccgcccatggaggctttcccctgggcgccccgctcgccccgccgcggccgcgcccccccgcccatggcgcttgtgcccagcgcccgctacgtgagcacccagggcccggcgcacccgcagcccttcagctcgtggaacgactatctgggactcgccacgctcatcaccaaggcggtggacggcgagcggcgcttcggctgcgcccgcggcggggacggcggcggagacggctccccgccctcttcttcctcctcgtcgtgctgctccccccacgcgggggccgggtccggggcgctggggcccgcgctggggccgcccgactacgacgaggacgacgacgacgacagcgacgaccaggggtcgcggagccgctacctggggggcgcgctggagctgcgcgcgctggagctgtgcgcggaccctgccgaggccgggctgctggaggagcgtttcgctgagctgagcccgttcgctggtcgcgccgctgccgtgcttctgggctgcgcacccgcggccgccgcggccaccgccgaagcagcgccgcgagaggagcgggccccagcgtgggcggccgagcccaagctgcacgccgccaacggggcggccgccacccggctgctcaaacccgagctgcaggtgtgtgtgttttgccggaacaacaaggaggcggtggcgctctacaccacccacatcctgaagggacccgacgggcgggtgctgtgccccgtgttgcgccggtacacgtgtcccctgtgcggtgccagcggcgacaacgcgcacaccatcaagtactgcccgctttccaaagtgccgcctccgcctgcagcccgcccgccgccgcgcagcgcccgggacggcctgcccggcaagaagctgcgctaagggcccggatccccatctgctgctgccccatgatgccaccagggtagacgcccgcccactctcgtgtttggtctgcgcaccatttctccctcgctgccgggggagcgtggagctcgtcttggcttttccagaggaagccaacggtactgagtattttcctgtcgaagagcggttgagactagacgttaaaatctttgattgtttctagtttgtgcacatccagacggtgaaggctgggtactaactgaaatgtggcaacttagaggcgctgtggtttgtttatacgtcgacctattttagatgcgcatcagtatgaaattgtctcaatctaatcttggatgtttaattttatgagtggaggcactttactaggtctagaatatttttttaaaagcctctcaactgaacttaaaactggcgattttatggagtgtcagcaaaatgactattttattgtttgaaacaagtaatatttctgttgtccttacccagttataattccaggtgaagccctgcctggtagcagcattaagtgaagacttggtatactttacagtagtttgggtgggtgttccctccttgtggcttgtttttgtcctagctggagatgtataaaatgtacaatttgtaggtagcaggtagaatacagctcatgtatgtaccagatctttttattactgaacgagcaactaccgttttccccctttaaaaatagtgccaagttataatcatattgtgtatacacttgaaaatggtgctgtttaaaaaaaattgtgtatttatacagtaacagtatatgaattcattaaccttgcctttaactcta

SEQ ID NO: 24 - DNA, 558 bp, Bos taurus
  source 1..558: mol_type = other DNA
agatctcagctgctcctgtctgcgggcccccagcccacttctctccagccacccaccaccaacactcccccgggtgccatgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccggccgcaaggtcaagcgctgaagaccgtcaggtacccacccgctgcagccccaaccctccctggttcagccctcccaagtcgac

SEQ ID NO: 25 - DNA, 548 bp, Bos taurus
  source 1..548: mol_type = other DNA
tcagctgctcctgtctgcgggcccccagcccacttctctccagccacccaccaccaacactcccccgggtgccatgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccggccgcaaggtcaagcgctgaagaccgtcaggtacccacccgctgcagccccaaccctccctggttcagccctcccaag

SEQ ID NO: 26 - DNA, 946 bp, Sus scrofa
  source 1..946: mol_type = other DNA
atgcagctgccaccctttgacatgtggaaggactacttcaacctgagccaggtggtgttgggactgatccagaatcgtcgacaagggccagaggccccgggcaccggggagccaagacctgagcccccactggagcaggaccagggcccgggagagcggggggccagcggggggctggccaccctgtgcaacttttgcaaacacaatggggaatctcgccacgtgtactcctcgcaccagctgaagacaccggagggcgtggtggtgtgtcccatcctacgacactatgtgtgtcccctgtgcggggccaccggtgaccaggctcacacactcaagtactgcccgctcaacggcggccagcagtctctctatcgccgcagtgggcgcaattcagccggccgcaaggtcaagcgctgaggaccgcaaggtgcccacccctgcacacccagcctctgggctcagtcctaccgagtccctggactcctctccattcccggactcaccccgccttctggaagatctgacccccaggatgccacccctgctggatcctgacaccatggaggctgtggggagccaggccagccctgccccttggacctctcagcccggggggttctgtactgggctgcttgtgaattcttggtgcggcgggacccctgccccttggaccacacagccctatggcttcttctggttcctggatcttttagtccttctgggacactagccctgccccctttgacaactcaggatcccactgtgctctcccaggcccacccctcagaatctggggagcgtgaacaggcaggcctgcgccctccagcccaagaacttcaagatgggattctgactcaagccagccgtcaccttcttcagccaccggcgacccaccggctcaccatggacgccaccggctgggtccctccctgccctcagcggcccg

SEQ ID NO: 27 - DNA, 3142 bp, Ovis aries
  source 1..3142: mol_type = other DNA
tctcctgcattggcaggtgggttctttacccctgagccagtgagacagtggaaagttaaatggagtctgagacagaccttggttgaatggatggttgagacattcagcattgctggtccctgggtcttacatctaatcacacctccaatcactgtatcaaacccccaaacaccccatcaattccaaaacatctcctgagggaggtcatagtctgagagcagcccctgcacactgggctggcctcatctgggaccctcactccattctagctaccccttcccgcttgctgtccccaaacttcatgccttcagagcttgtgctccttttcctagaagattgttccatgtggtctcaacttccaggtcccctctcccagtccaggctatgaattagtagttggctctttatggtctattgtttctcatttgctggctgttccctcattggactgcttgttcacgacacaggcacaaagtcgctgtggccactgctgagtttctagaacccagtataaggggcctcaatgatttctgtgttgagtgatccaggggaccaactgggaaccacagatggaggaaatattcctggaggtgacatgggcaggagacatgaaggtcaggaaggatctgggacaatataccagcccaagggggaaaggtgggcattctaagggagactgtgccataccaaggttctctgctgcttttcagatctattcccagtgccattctctgggggcagacctgggtgcaaatccctactctgcaattttcctgctgtgtgaccttgagtactctccttaacctctctgagcctcatatccttttctataaaatagaaacgatggtatccgcctccttgagtggttacaaggttcagagagatcatttttgcaaaacattgaacactccagtgtgcacttagtagatatttggtgagtaagtggctcctagttccagatgtgatgaaaaatgaaattttaaccctaaataggttggaggagcattgagctgtggagtcacatggcctcaatttaaagccccagctctgccacatacctgttatgtgactctgggccactgagttttcctctctgagctttagtgacttgtcaataaaatgggatcataagtgtgttacccgcctggcattgctgtgaaaatgagatcagattgtgaatgagactgtgcggcacagtgtctggctcctggtaaggggttatgttgttatgattgggaatggctctgcagtatatggagggagcaggcccccagccctggaataagggaggatgggggtgggggcatttgcagacatgaagttttgttgaaaggacagaaaaactgaggcccagaaaaactgaggcccaggtcattccagaggtcacagagcaccttgcaggcccagcaagcaccagagacaacaggaaagaccgccttccagatctccatggagaattaaagtcagaagggtctcggtaaggagtgggccctttaaatcctaagctccacctttgcttagaagggtctttgggaatataaaagggggtgcagttcctctccactcctgaaaaccatcagctgctcctgtctgcaggcccccagcccacttctctccagccacccaccaccaacactcccccgggtgccatgcagctgccaccctttgacatgtggaaggactacttcaacctgagccaggtggtgctggcactgatccagagtcgggggcaggggctggagacccaagggactggggagccgagacctgggccccatgtggagcaggatcaggggcagggaggacgggggactggcgggggcctggccaccctgtgcaacttttgcaaacacaatggagagtctcgccacgtgtactcctcacaccagctgaagaccccggagggcgtggtggtgtgtcccatcctgcggcattatgtgtgtcccctgtgcggggccaccggggaccaggcccacacgctcaagtactgcccgctcaacggggggcagcagtctctctatcgccgcagtgggcgcaactcagccggccgcaaggtcaagcgctgaagaccgtcaggtacccacccgctgcagccccaaccctccctggttcagccctcccaagcccctggactctccccatctctggactcttccggccctttggaacgtctgactctcagtactcgatccctgctggatcctggaacaacgcaggcagtgggaaggcaggccagctctgccgccccttagaactctcagctctcgggactctgggagtgtttgtggattctccggtgcagcgggatcccctactccttggaccacacagccctgtggctcctcctgggccacagtccatggatctttagttcttctgggacgcttgccctgcctgacttctcagccctcagggttccattccttggggaattcttggaccgtcgggcttgcaggacttcgctgtgctctcccagggacacccctgagaatctggagagagtcaacaggctggcgtggaccttccagcacaagaactttaacatgggattctgacttaaaccagccttctccgcctccttcgggccaccggcagacccatcgtttcatcgtggacggcaccggatgggtcccaccctcagcatcctatccatcaagagctgggggaaggaggggtcgcgtcagagggacaccacgtcttcgcaagcttgtctggggctggagagccttggggtgagccagtacccttgaactcccccccacccccgctgttcttcccatcgttcattccttttactgcgctcctgcccccacggtgatggcaaatcaccgacgggtcttctccgtagcgagagggcatctctccgccacctcccacccgcccccacaaatgcccccagtttgtaaaatggtcctttctgtcctcccgtgtttttttcaacctccaacagtttagtaaacgaagctcccactagaaaggggttttaatgcccggaggggaatggcaaccgcctagattttattttaggggcaaagaaaaagccccagtcagatttttaagttgtttggtttttagctcagggctgggcccgggtgttgcgctttagtgaataaacaaaacgctttgctggttgtctttgaaa

SEQ ID NO: 28 - DNA, 283 bp, Bos sp.
  source 1..283: mol_type = other DNA
tgggaggcggcggccgcgggttcgagccggcgccggagccccgcggtcccctccccctgcccgcggcctggggagcccccgcccagccccggagccgccaaaatgcaatttcccgtgccggcgcctcgcggctcggggggcttttccgggcgggttttggacagaagagggggaaacaaggcggcggccccaaaacgaggttccaaaggtgccaggaagtcttctggaactcctccctcttcctgctgcccctcaacttctgcctaaggagactggcgtgggc

SEQ ID NO: 29 - DNA, 822 bp, Ovis aries
  source 1..822: mol_type = other DNA
ctcttcacacccccttggagtataaggagggaactgacagcccagactcctctgctccagagagggaagggaagggagaggaggcaggggtagagagaccagcttggaggcggctgggtttctcctctcttctgcccctccgcctggcacacggggccaagccatggggaccttcaacctgtggacagactacttgggtttggcacgcctggtgggggctcagcgtgaggaggacgagccggagaccaggctggaccgccagccagaagcagtgccagaaccggggggtcagcgacccagccctgaatcctcaccagctcccgaacgcctgtgctctttctgcaaacacaacggcgagtcccgggccatctaccagtcccacgtgctcaaggatgaagcaggccgggtgctgtgccccatcctccgcgactacgtgtgcccccagtgcggggccacccgcgagcgcgcccacacccgccgcttctgcccgctcaccggccagggctacacctcggtctacagctacaccacccggaactcggctggcaagaagctggtccgctcggacaaggcgaggacgcaggacactggacacggatcgcgccgaggaggaggcggcggaggaggtgcctgtgcaggtggctggggggactcgctgcacagggggttctgccctgggctgacttagagcctctgagaagtgggttaacccctgggctgacccacttcagagggttgggtgggggacagaatccatacataatagaaagcttagaactgtgttctagaactgttgccccagtgggtaagctagggctggga

SEQ ID NO: 30 - DNA, 3539 bp, Ovis aries
  source 1..3539: mol_type = other DNA
gaggctggctctaggtggggatctcgagggtggggctccgagcgccgcgctgcctctggccgcctccgcggaaggtcatttttgggttcactcaagctcagcacacctgggggctctggtttgccttaagtgttacgacaagcggttcatcctttggggctcgatactgccctcagtgaaatggaaggggactggaaatttgaagtccaggccgttctctggcgagggggtgggtcccagacctcccagacccagggaggtggggagcagcctcccctgagactgcgcgtgggggcaccctagggggtgggcggctaactctacagcggtccctggagggagggactggggaccgggtttgagggtgaagaaatggggaagggcagtaacggggcaaccctcgcgtggttatgcgcttgggcccgggtctgatcccgacaagggcctgtttggaagcccgggaccctctgcgatcctctagcttcgcccttgtccaaccggcagttggatccacaggcgggctgggcagcggccccacctcggggctcgaatttgcaaagtgcagactctgaccacccacccccccccaccaccgtgggttgttgtgattataaaccattgtgtccggaacacggtgaagctcactgaaggtattacattgtattaaaatgacttgtttatctctccgttgcatccatgcccccggggccagaaccacttggcctccagacctcttggggccactccgaatccctcccctgcctctcgctaagggtgccctctggcctgtttcccaaactgacaattggaagaaatatgcacctttgagggcccttttgagaatgctttgactaaatgggttagaagcccagcacccactgctgctatatttgcatagcaagggtgacagaagtatctgctgttactagtacttagatttatctcctttttcggtgtcctggagcagggagttggctcctgctgttttctaacttaatagattctctaagtctctcattgccttcccctctcctccctcaccctatccggttccttgaccccctcaacctccactccccacccccaaggagttgccaagggtttgggggaacattcaacctgtcggtgagtttgggcagctcaggcaaaccatcgaccgttgagtggaccccgaggcctggaactgccgtccacccacccatcacgacccccaactttcagatctggggcaggggcaggggatcccgaatacatcccctcccttaggccacagcgaaggtcacaatcaacattcattgttgtcggtgggttgtgacggagaccagacccaccgagggatgaatgtcactgtggctgggccagacacagcctcgggggaatgggatggaggatacaacccaccccggactcccctcctcccgcccccctccaaactgctcagccagaacctgaccctgaccctggcctctcacccctcgaggagggctggtgtctggaatccttacagacacaggctggatttgggggactcagtcctggagggctgtgaacaggaattacaagtttaggactgggcagctgaaaaacctttctgaaagggattaggggtccctgcttccagaaggctcagtgaagctttcttgaatgaatgaatgaatgagtgaatgactgaggtgtataggtggcacatcacctcttctctgagttccagtcttgggccctgctttctcaccccccttacctggtacctgcagacccctcctttaccttcagtcgcccacctagcccctgatgcctgttgatcacctgccagtctgtgtcccacctgggtgactcaggggcacactgcatcctcctgagatggagcacaggtctcatttgagagggtgatcaaggtgctggcgaatgctaggggtctcccctctggcccgttagccccacctgcgcctgtgctctcttcttccccataattctcagtctcaaacccttttccaccccaggaccaggagagactgaccccacaacacctaaggctcctgtaactggtaggggaggcaggctttgtgtttgtctttgtgaataacctcagggcagatggcttcaaaccccagtttgttcatcagttaaaaggaggttccactgacaaggggtgtgaaagctccctgagggtgaccagagctacgggccttggtcctgtcccccccaaccccataagtcaggcccatcctccttccaaagacagctggaacatcagcagctccccctccccctcccccggtccaggagaaggaagaaagaagttactaagttactggctacagcaatgctagtctttggggtggggcttccaatgcccccacctgcatcactctggttctctaggaggcttagacaagggtagccctctcaatgccctctgggtggggtgtgtggctgcttattgctggtgccccctgcagcctgtgtcttgtcacgccccctcacccctggcctacccagaggccatgcagccccgtggcaggtgcatttctgggggagctgcagcaagccccctgtgatgatagggaacctcctacagcctgctcctccctcttcacacccccttggagtataaggagggaactgacagcccagactcctctgctccagagagggaagggaagggagaggaggcaggggtagagagaccagcttggaggcggctgggtttctcctctcttctgcccctccgcctggcacacggggccaagccatggggaccttcaacctgtggacagactacttgggtttggcacgcctggtgggggctcagcgtgaggaggacgagccggagaccaggctggaccgccagccagaagcagtgccagaaccggggggtcagcgacccagccctgaatcctcaccagctcccgaacgcctgtgctctttctgcaaacacaacggcgagtcccgggccatctaccagtcccacgtgctcaaggatgaagcaggccgggtgctgtgccccatcctccgcgactacgtgtgcccccagtgcggggccacccgcgagcgcgcccacacccgccgcttctgcccgctcaccggccagggctacacctcggtctacagctacaccacccggaactcggctggcaagaagctggtccgctcggacaaggcgaggacgcaggacactggacacggatcgcgccgaggaggaggcggcggaggaggttccaaaggtgccaggaagtcttctggaactcctccctcctcctgctgcccctctacttctgcctaaggagactggcgcgggcaggatgacgccttcacctggggatggggacccagccggggtttcagggaagacccaccctccgaggatccgcccctagacggtgccttcagcccggggacttggcaaaggagccgggtctgggaccaccgcccaaagcgcgcccgaccccgtcactgaagggggtggtcctcaggcacccctgcccttcttccccagcgctgggcacccagtcagcgctcaataaatgtttatgaatgga

SEQ ID NO: 31 - DNA, 3551 bp, Ovis aries
  source 1..3551: mol_type = other DNA
gaggctggctctaggtggggatctcgagggtggggctccgagcgccgcgctgcctctggccgcctccgcggaaggtcatttttgggttcactcaagctcagcacacctgggggctctggtttgccttaagtgttacgacaagcggttcatcctttggggctcgatactgccctcagtgaaatggaaggggactggaaatttgaagtccaggccgttctctggcgagggggtgggtcccagacctcccagacccagggaggtggggagcagcctcccctgagactgcgcgtgggggcaccctagggggtgggcggctaactctacagcggtccctggagggagggactggggaccgggtttgagggtgaagaaatggggaagggcagtaacggggcaaccctcgcgtggttatgcgcttgggcccgggtctgatcccgacaagggcctgtttggaagcccgggaccctctgcgatcctctagcttcgcccttgtccaaccggcagttggatccacaggcgggctgggcagcggccccacctcggggctcgaatttgcaaagtgcagactctgaccacccacccccccccaccaccgtgggttgttgtgattataaaccattgtgtccggaacacggtgaagctcactgaaggtattacattgtattaaaatgacttgtttatctctccgttgcatccatgcccccggggccagaaccacttggcctccagacctcttggggccactccgaatccctcccctgcctctcgctaagggtgccctctggcctgtttcccaaactgacaattggaagaaatatgcacctttgagggcccttttgagaatgctttgactaaatgggttagaagcccagcacccactgctgctatatttgcatagcaagggtgacagaagtatctgctgttactagtacttagatttatctcctttttcggtgtcctggagcagggagttggctcctgctgttttctaacttaatagattctctaagtctctcattgccttcccctctcctccctcaccctatccggttccttgaccccctcaacctccactccccacccccaaggagttgccaagggtttgggggaacattcaacctgtcggtgagtttgggcagctcaggcaaaccatcgaccgttgagtggaccccgaggcctggaactgccgtccacccacccatcacgacccccaactttcagatctggggcaggggcaggggatcccgaatacatcccctcccttaggccacagcgaaggtcacaatcaacattcattgttgtcggtgggttgtgacggagaccagacccaccgagggatgaatgtcactgtggctgggccagacacagcctcgggggaatgggatggaggatacaacccaccccggactcccctcctcccgcccccctccaaactgctcagccagaacctgaccctgaccctggcctctcacccctcgaggagggctggtgtctggaatccttacagacacaggctggatttgggggactcagtcctggagggctgtgaacaggaattacaagtttaggactgggcagctgaaaaacctttctgaaagggattaggggtccctgcttccagaaggctcagtgaagctttcttgaatgaatgaatgaatgagtgaatgactgaggtgtataggtggcacatcacctcttctctgagttccagtcttgggccctgctttctcaccccccttacctggtacctgcagacccctcctttaccttcagtcgcccacctagcccctgatgcctgttgatcacctgccagtctgtgtcccacctgggtgactcaggggcacactgcatcctcctgagatggagcacaggtctcatttgagagggtgatcaaggtgctggcgaatgctaggggtctcccctctggcccgttagccccacctgcgcctgtgctctcttcttccccataattctcagtctcaaacccttttccaccccaggaccaggagagactgaccccacaacacctaaggctcctgtaactggtaggggaggcaggctttgtgtttgtctttgtgaataacctcagggcagatggcttcaaaccccagtttgttcatcagttaaaaggaggttccactgacaaggggtgtgaaagctccctgagggtgaccagagctacgggccttggtcctgtcccccccaaccccataagtcaggcccatcctccttccaaagacagctggaacatcagcagctccccctccccctcccccggtccaggagaaggaagaaagaagttactaagttactggctacagcaatgctagtctttggggtggggcttccaatgcccccacctgcatcactctggttctctaggaggcttagacaagggtagccctctcaatgccctctgggtggggtgtgtggctgcttattgctggtgccccctgcagcctgtgtcttgtcacgccccctcacccctggcctacccagaggccatgcagccccgtggcaggtgcatttctgggggagctgcagcaagccccctgtgatgatagggaacctcctacagcctgctcctccctcttcacacccccttggagtataaggagggaactgacagcccagactcctctgctccagagagggaagggaagggagaggaggcaggggtagagagaccagcttggaggcggctgggtttctcctctcttctgcccctccgcctggcacacggggccaagccatggggaccttcaacctgtggacagactacttgggtttggcacgcctggtgggggctcagcgtgaggaggacgagccggagaccaggctggaccgccagccagaagcagtgccagaaccggggggtcagcgacccagccctgaatcctcaccagctcccgaacgcctgtgctctttctgcaaacacaacggcgagtcccgggccatctaccagtcccacgtgctcaaggatgaagcaggccgggtgctgtgccccatcctccgcgactacgtgtgcccccagtgcggggccacccgcgagcgcgcccacacccgccgcttctgcccgctcaccggccagggctacacctcggtctacagctacaccacccggaactcggctggcaagaagctggtccgctcggacaaggcgaggacgcaggacactggacacggatcgcgccgaggaggaggcggcggaggaggtgcctgtgcaggttccaaaggtgccaggaagtcttctggaactcctccctcctcctgctgcccctctacttctgcctaaggagactggcgcgggcaggatgacgccttcacctggggatggggacccagccggggtttcagggaagacccaccctccgaggatccgcccctagacggtgccttcagcccggggacttggcaaaggagccgggtctgggaccaccgcccaaagcgcgcccgaccccgtcactgaagggggtggtcctcaggcacccctgcccttcttccccagcgctgggcacccagtcagcgctcaataaatgtttatgaatgga

SEQ ID NO: 32 - DNA, 4924 bp, Bos taurus
  source 1..4924: mol_type = other DNA
atgttggatatttgcttggaaaaacgtgtgggtacgaccttggctgcccccaagtgtagctccagtgctgtgaaggttccagggttggcagaggggactgaagggaccatgaaaatggacatggaggacgcggatatgactctgtggacagaggctgagtttgaggagaagtgtacatacattgtgaatgaccacccctgggattctggtgtggaaggaggcacttcggttcaggcggaggcttccttaccgaggaatctgcttttcaaatatgccacaaacagcaaagagattactggagtggtgagtaaagaatacataccaaagggaacacgttttggacctctaataggtgaaatctacaccagtgatgcagttcccaagaacgccaacaggaaatatttttggcggatctattccagaggggagcttcaccacttcattgatggctttaatgaggagaagagcaactggctacgctacgtgaacccagcgcacactgcccgggagcagaacctggccgcctgtcagaacggcatgaacatctacttctacaccattaagcccatccctgccaaccaggagcttcttgtgtggtactgccgggactttgcagaaaggcttcactacccttattccggagagctgacaatgatgaatctcacacaaacccagagccgtccaaagcagcagagcactgagaaacatgaactgtgcccaaagagtgtcccgaagagagagtatagcgtcaaagagatcctaaaattggactcccacccttccaaagggaaggacttgtaccgctccaacatttcacccctcacttcagagaaggacgcggaagacttcaggaaaaacgggagcccggagatgcccttctacccgcgcgtggtgtacccgattcgcgccccgctcccggaagactttctgaaagcacccctgggatgctacgggatggagcggccgccctacatcactcactcccccatatcgtcctccacgacgcccagcccctcggcgcgcagcagcccggaccggagcctgcagagctgcagcccgcacagcagccccgggaacacggtgtcgccgcgggcccccggcccccaggaggcccgcgactcctacgcctacttgaacgccccctacggggccgccgccgaggggctgggctccctagccggctacgggccgcccacccacctgccgcccgccttcatcccctcctacaacgcgcactacccgaagttcctgctgcccccttacggcatgaactgcaacggcctgggcgccgccgtgagcaacatcaacggccttaacagcttcggcctcttcccgaggctgtacccggtgtacagcagcctcttgggcgggggcggcctgcctcccccggtgctggcgtccgcctcgctccccagctcgctgccctcggagggggcgcgccgcctgctgcagcccgagcacccccgggaggtgctggtgcccgcgccgcacagtgccttctccctccccggggcggccgccagcctgaaggacaaggcctgcacccccaccagcggctccccgacagccgggaccgccgccacggccgagcacgtggtgcagcccaaagctacctcagcggccgctgcggcccccggaggcgaggaggcggtgaatctcatcaagaacaaaaggaacatgactggctacaagacactcccctacccgctgaagaagcagaacggcaagatcaagtatgagtgcaacgtctgcgccaagactttcgggcagctctcaaacctgaaggtccacctcagagtgcacagtggagaacgacctttcaaatgccagacgtgcaacaagggcttcactcagctcgcccatctgcagaaacactacctggtacacacgggagagaagccacatgaatgccaggtttgccacaagcgatttagcagcaccagcaatctcaagacccacctgcgactccactccggagagaaaccttaccagtgcaaggtgtgccccgccaagttcacgcagtttgtgcacctgaaactccacaagcgcctacacacgcgggagcggccccacaagtgcgcccactgccacaagagctacatccatttctgcagcctcaaggtccacctgaaggggcactgtcctgtggccccggccactgggctgcctctggaggacctggccagaatcaacgaagagattgagaagtttgacatcagcgacaatgccgaccggctggaggacatggaggacaacatcgacatgacctctgtggtggaaaacgagattctggccgtggtcagaaaagagaaggaagaaactggcctgaaagcgtctttgcaaagaaacctgggaaatggactgctctcagggtataacctctatgagtcaacagagccgtccctcctgaagttgcctcacagcaacccactacctctgggacctgtaaaggtcaaagaagaaacagtcgaacctatggatccttaagattttcagaaaacaaaaggtgttttgtcttgtttcttaacttacgacttggcgagtcagggtgcctgtcgcaagcgcttgtacataattccaattctgcaaagctctccctgacggcagatggtttccctcacctctctggaattaaagaaggaaccccaaagttactgaaatctcagggcataaatgaggcaaagacaatatatatacctattatatatacttatttacaccccatctatatatttgaacctgttgtattttgaatatttgtgtgaatatgtttgcatagcctccccattgctaagactattgcctagccataattattttttcaatgataatccttcataatttattatacagtttatcattcaaaaagcaataattaaaaaaagtttacaatgactggaagaattccttgtaatttgagttaagtgttatatttttgtctggtggccattctttgtagataatttctgcacatctgttttaagtatcttaagatttaataaacagatacattacttcaagtcaacctccctctataataatggtttgaaagtgaggtttgagtattgccaaagttaggcagttgatgtgttcattcataggattatattatttgaacagccttgtatcatttgaacataatttgggggtatgtttgcagaattaaccagtaataggtggaagaaataaaaaccttgtatgtttctgtagagagttcagcattcctgcccccaatcacagatttaccagaaaggtgataaggcgttgccagtctgtctccctcttcaaaaaaacagggttgtcccatccccaaagtcatgtgaccatttgcagtggccatgagactttagcatcccaatgtattatctgaaaatgtgaagaagacaaatgccatgtttaaaaccactgcaaaatgtttccaaagcacaggtggttttgtatgggtgaggtctgggggcatgagtccaggtgtttttgttgttggtcttttgttattacgtcagaattgcacaagcatggtgctccaccaggaaggatttgagttagataggctcaggccacactttaaaaaccaaaagccagaaaacgggtattctcgttgtcttagggtcaaagcgggtaatgaacattcctgtccccaacacatcagttgtatttgttttttctgtgcaactcaagacttttcctcagtatttgtgtttttacattttatggttaatttaattgaagaagaaagggcattgcaaagttgttcaacaacaattacctcattgagtgtgtccagtagtgcaggaagtgatggcatctttaatgatttgctactcaagaggagagagcacgtgtgcaccagagagatggaatgcatcccattctgcctcgtcctctgctaagcccaaagataacacgttggcattcaaggtgtagcggacaatgatgtatatttataaatctatttataccactataccatgtatatagaatatatttataaccacttaaaattgtgagccaaaacatgtaaaagatccttcttttcctaatggcaaaaaaaaaaaaaaaaaaatcaaaccctctctgactctttgcttaattagcactcgatgacctcacaattacattggtgagcttgggcacccccttggtattataagagatcctaaaattttggtgcaaaggtggggaccgcataacaaccagggagaaagagacataccttttttaactattaaggacaatctaagatggcttttttttttttttttttgtcacattctgatgtggtcacacccaagtcccagaaatagaaatcccatcaaccactgtaacttttctctcttcctctccttccttcttaaataactgatggatcactaatgaatctggtttatatattcagcattttggtctgtggcaactagtatcagcctacagaagagaacaacaaatgtaggataatccagggaaacctaagagccttactgatctgttcctttaaataatgaaactgacagttttttttaatattttttttaacagtgtttgacaaacagtccttgattataagtaaaccaaagcatcatactgacactgacatcaaatattttctattccccatcactcatttgttctcttttttccctctcatgctttctcattagtggaaatttttactgagacctgcacagtggcttgagaccccgtaattctctaccatctgactttccctctgaggcctcttacatgtgaatattgagcccacaatcaagagtggttttgtttttttctttcttctactcaaaactgaccaaagttactggctttttactttgctagaacaaactatcttatgtttacgtactggtttacattgttatttatgtgcaaattgtcaaaatgtaaattaaatataaatgttgatgcttta

SEQ ID NO: 33 - DNA, 5740 bp, Sus scrofa
  source 1..5740: mol_type = other DNA
aaggacttcgacagtgttatgtttacattctaacgaggacacgctcttacatggaaatgctaccctcgttccgacttttcccccaacgcgttggtatgggaaaactgcggacggtgtttacctctcttctctctctctctctctctctctggaaagcactcttcctgcgtgtgaggaggatttgcgggaatacccttctcaatttacaagtggaagcagagctcgctttctcagtcttctgtatttgttataactttagttacagtaaactgtagcccatcagcgacttctgggaattcgtacactttcagatttaaatggaaagtgctatttgtagccgaggactcctaaaggaattctctccagggaattctattgaacagtcttgtgttcggttttttgctttttcaagatgcccacggtcaaaagaaactgcggggagttgtcgcgcccccctcccccccatctttcctccctcttttcctatggtccaaatgtttctgaaaagatgcagctcagtgatgctggcaggatgcaacccttttcagccttccatgtgagaggatgaaagagatcgcgctgcagagggggttgggggggggagttgttttgaaagttgttttgcgttgggagctggtgggaaagttcagtcttccccatttggaaaagtcaggctgggttccgctcctgcaccacacgcgctttccatttttagcttcattcagaggcagacagagctccttcctcttcctctccttgtttgtgacacttttctgaggcagcttttccacagcgctgagggtctggcggccatgaccccaggcgtccagggacagaggactgggcgcctacagcatttttctgccctgagaggtaaagctgggaattgttcagaggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 34 - DNA, 5826 bp, Sus scrofa
  source 1..5826: mol_type = other DNA
gttccacattccctttaactaggaaacattaacgctctacactttgctcacacactcctgggaagtctgcctgtgggtttgggggaggagagaaaaggcaaagacattttaatatgtaggattataacttcttcctatttccctctttcctaaatagttgaaagaaggaagtcagttctactaaatttgagtgcgcttttagttagcaagagaaatggaagcttacaagtaggggaggagaaaatgggtatttcacaggcaatagcttgaggactagacagctccatctttattacagtcaatgcaaaaacaattgctcttttcacgtgctctcttcaagtaaattccgtgtgttttgtaaccggatctgcgccgaggaacaacgccaacagttgtaggtaatttttcggtttccccgaatttcaggaatgtgcgcccccttccaaagccgcagcaggaagaagggcggtctggagtatttagaatacaatgcggcccgagttaacgctgaggttaagcgccttcaaagggaagtcagaagattccaagtcaatgttgaaattcgcagagcagagaggaagcggcaggcgctggcgagctgggggaaagccctggccgagcgcggcgcggcgcggccgagtccaggtgtggccacgcccccacttcaggcagctggtaggagtgaatcagaaatccgcagcagggaggggaaagggccgagccgccggccgggttaacagacagaaagtgctgccgtgacactcggccctccagtgttgcggagaggcagagcagcgcggcacctgtccgcgtcgggagccgggacgcgggcgccggcggccggagcagagggcaccgcggcggtgcgcgacagccctcggcggggaggggaacgcggagcacatgcggactgtgtagggatgagcaacacttatctccgatgttggatatttgcttggaaaaacgtgtgggtacgaccttggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 35 - DNA, 5614 bp, Sus scrofa
  source 1..5614: mol_type = other DNA
ctgaaggacttcgacagtgttatgtttacattctaacgaggacacgctcttacatggaaatgctaccctcgttccgacttttcccccaacgcgttggtatgggaaaactgcggacggtgtttacctctcttctctctctctctctctctctctggaaagcactcttcctgcgtgtgaggaggatttgcgggaatacccttctcaatttacaagtggaagcagagctcgctttctcagtcttctgtatttgttataactttagttacagtaaactgtagcccatcagcgacttctgggaattcgtacactttcagatttaaatggaaagtgctatttgtagccgaggactcctaaaggaattctctccagggaattctattgaacagtcttgtgttcggttttttgctttttcaagatgcccacggtcaaaagaaactgcggggagttgtcgcgcccccctcccccccatctttcctccctcttttcctatggtccaaatgtttctgaaaagatgcagctcagtgatgctggcaggatgcaacccttttcagccttccatgtgagaggatgaaagagatcgcgctgcagagggggttgggggggggagttgttttgaaagttgttttgcgttgggagctggtgggaaagttcagtcttccccatttggaaaagtcaggctgggttccgctcctgcaccacacgcgctttccatttttagcttcattcagaggcagacagagctccttcctcttcctctccttgtttgtgacacttttctgaggcagcttttccacagcgctgagggtctggcggccatgaccccaggcgtccagggacagaggactgggcgcctacagcatttttctgccctgagaggtaaagctgggaattgttcagaggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 36 - DNA, 5716 bp, Sus scrofa
  source 1..5716: mol_type = other DNA
aggacttcgacagtgttatgtttacattctaacgaggacacgctcttacatggaaatgctaccctcgttccgacttttcccccaacgcgttggtatgggaaaactgcggacggtgtttacctctcttctctctctctctctctctctctggaaagcactcttcctgcgtgtgaggaggatttgcgggaatacccttctcaatttacaagtggaagcagagctcgctttctcagtcttctgtatttgttataactttagttacagtaaactgtagcccatcagcgacttctgggaattcgtacactttcagatttaaatggaaagtgctatttgtagccgaggactcctaaaggaattctctccagggaattctattgaacagtcttgtgttcggttttttgctttttcaagatgcccacggtcaaaagaaactgcggggagttgtcgcgcccccctcccccccatctttcctccctcttttcctatggtccaaatgtttctgaaaagatgcagctcagtgatgctggcaggatgcaacccttttcagccttccatgtgagaggatgaaagagatcgcgctgcagagggggttgggggggggagttgttttgaaagttgttttgcgttgggagctggtgggaaagttcagtcttccccatttggaaaagtcaggctgggttccgctcctgcaccacacgcgctttccatttttagcttcattcagaggcagacagagctccttcctcttcctctccttgtttgtgacacttttctgaggcagcttttccacagcgctgagggtctggcggccatgaccccaggcgtccagggacagaggactgggcgcctacagcatttttctgccctgagaggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 37 - DNA, 5244 bp, Sus scrofa
  source 1..5244: mol_type = other DNA
tgtttttaaatccgaaaactttttttttttttcgtgctaacgcagtcgcggtagacaccagaaatgcttgtgctttagggcacttggtgctgtcaaacatttataagactttttccttatgaatcattaaccctttcagttctaggcataattgtcagttcattgaaatttcagtagtggctcccgcccaccccaccgtcacacgaagtgactattttgggtcggcttcatttaaattttttgttttattttaacatttatttggcatcgtgactatatctgtggcaaccacatagacgtgagcacttttaaatcgtcacggagggaaagacaacttcgcatccgacttttttttttttttttttttttaagagcatcatattcagggaagaactagaaggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 38 - DNA, 4944 bp, Sus scrofa
  source 1..4944: mol_type = other DNA
gggtttctgagaggcgagctgtttggatcattcatgatttgctgccattgtgaacactcttaaggagagaactctgcctgagaaatgctgattaataaaggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 39 - DNA, 5706 bp, Sus scrofa
  source 1..5706: mol_type = other DNA
cagtgatcagttccacattccctttaactaggaaacattaacgctctacactttgctcacacactcctgggaagtctgcctgtgggtttgggggaggagagaaaaggcaaagacattttaatatgtaggattataacttcttcctatttccctctttcctaaatagttgaaagaaggaagtcagttctactaaatttgagtgcgcttttagttagcaagagaaatggaagcttacaagtaggggaggagaaaatgggtatttcacaggcaatagcttgaggactagacagctccatctttattacagtcaatgcaaaaacaattgctcttttcacgtgctctcttcaagtaaattccgtgtgttttgtaaccggatctgcgccgaggaacaacgccaacagttgtaggtaatttttcggtttccccgaatttcaggaatgtgcgcccccttccaaagccgcagcaggaagaagggcggtctggagtatttagaatacaatgcggcccgagttaacgctgaggttaagcgccttcaaagggaagtcagaagattccaagtcaatgttgaaattcgcagagcagagaggaagcggcaggcgctggcgagctgggggaaagccctggccgagcgcggcgcggcgcggccgagtccaggtgtggccacgcccccacttcaggcagctggtaggagtgaatcagaaatccgcagcagggaggggaaagggccgagccgccggccgggttaacagacagaaagtgctgccgtgacactcggccctccagtgttgcggagaggcagagcagcgcggcacctgtccgcgtcgggagccgggacgcgggcgccggcggccggagcagagggcaccgcggcggtgcgcgacagccctcggcggggaggggaacgcggagcacatgcggactgtgtagggatgagcaacacttatctccgatgttggatatttgcttggaaaaacgtgtgggtacgaccttggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 40 - DNA, 6916 bp, Sus scrofa
  source 1..6916: mol_type = other DNA
tcatagcaatgatgaatcccagacccactgagcaaggccagggattgaacctgcatggtcatggatactaatcgggtttgttactgctgagccacaatggaactcccaaaatatatatttgcataacatgtacataattactgggaaaccattatttaaattcttttaaggaacatgtacatggtgacaacaaaaattgcattacacttactctggtcagtatttacctagaacgcttatatctttaggacaagtatctttataagagacatatttggggatcattcagaaactgttcctaaggaacacaattttttttttaatccaggaagttgtgtgtttttagaaatactttagtatttcccaaactgagaattgtccactaagaattgtgtgctcacaaattttatttaataattactcatgcttaaagtatatttaatacctgaatatttgatacctggcaaaatgggagtgcattttcacctttcttgccagtaaggtcttctcagaagtgagctatcgtgatgtgtaattcttgggagtggaaggtcagaattaagacaatccagcccaagtctctttaatgcgtgattcttttgaaaaaagacaagaaatacttacaaacctcctatctctgtcccagttctctggttcatggaagattgactctgagcaactgaagtgctttccttgggaggatttctttgaagcaggtctttctttcacgtgcaaaggatattctagggagataacttcccactgcttgttcaaagcatcctgattttgctggcaggaatatgaacatctgttgtggggaaagaaaaagtttcatgcaaattacacagtcaaagaagattagggatgttcaagttgagaaaccagtgacatttcttgtaactgtgcaatgagtcagcacgtttttaatcttcttgataatatatggaaatgctgtgagggtgagagggagcggcatccacctgacgtatcattactttattttcttgtgggtgtgtgtgtgtgtgcgtgtgtgacttcatggcatcgacatttctgtttttaaatgaggatacagtaaattgtagtccgtggaaggctgactggaatcaacacagccgtagctttagaaagcagtttccgcccagcgaagtgcagagcgatggaaccccatgttcctggaagtttgcacatcagagtaaacaaacttgaaaacccctcttgatagcagaattcacccagccttgttccattttctcttaacaaaacacaccgcaaaagctctcacaagctgctttgatgaagccacatgtatttcccccttcacaatttacaggaagttactcttaaaagaaagtgattctggtgtttactgcccgtgttaaagggacagagttcctttttgtctctgataacgctgacgcgaaatgcagaaagaatttatacacgaacataatcagtaggtgactaaggacaaagccatcacctgctgtctggttagagccaaacttctgctatgagccctgcccaccggcttggagcccccaagttgcagctaagcaggtactgagtgtgtagatgcagggagctttcttaagtctgaagacaaggtgggtacatcttaaacggagggaaagagaagcttttctgtggaaggataaggagtttgtttataaagccggctcaggaagaatgtagatgttcatttctcctattccctctgccttttcaccgagtggcctgagggcatctgttaaccagaattactcttactctagggcaaaccaaattcttcattttggttgctgtcttgaaagaataagctggttgcttccagttctttttccaaacaccccaggcctgtggctctgtgagtggtgtcttagccaacctcctctcctctcctttggctttgaaagaaagctcaaggccaaagctgtttatcaagaggaaaagtgacttctcagtagactgtcaacttttggcttctgtcccggtgttcgttttgttgtggcaggtgaagttcaccttggccccacccagtgtttccacaaaaaggcaaggttccaagtattcagatgaacaagtgtcactgctggacttggaggcttgggggtggaggatgtttgcatagttttagccttgctcgggcaggcgggggtgtaggaaacgggcaagtacagaggccatagaaaacgttgagagactcagtttgacgtactcagtcaggctggtccaatacccagtgactcaaagcattaaaagtcagcataatcggaactcaagttagtggcatcgcctgtgagccattcactgcgcagtagcagaaatagtactaggtggtggtttaatccttctgaagggagctctttaaacgacattttgtttcattctccccccccacgcccccaacatgaaaagacactaaaactgaaatggaaaagatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 41 - DNA, 5010 bp, Ovis aries
  source 1..5010: mol_type = other DNA
gtttgtgacacttttctgaggcagcttttccacagcgttgagggtctggcggccatgaccccaggcgtcctgggacagaggactgggcgcccacaacatttttctgctgtgagaggctgcccccaagtgtagctccagtgctgtgaaggttccagggttggcagaagggaccgaagggaccatgaaaatggacatggaggacgcggatatgactctgtggacagaggctgagtttgaggagaagtgtacatacattgtgaatgaccacccctgggattctggtgtggaaggaggcactgcggttcaggcggaggcttccttaccgaggaatctgcttttcaaatgtgccacaaacaccaaagagattactggagtggtgagtaaagaatacataccaaagggaacacgttttggacctctaataggtgaaatctacaccagtgatacagttcccaagaacgccaacaggaaatatttttggcggatctattccagaggggagcttcaccacttcattgatggctttaatgaggagaagagcaactggctgcgctacgtgaacccagcacactctgcccgggagcagaacctggccgcctgtcagaacggcatgaacatctacttctacaccattaagcccatccctgccaaccaggagcttcttgtgtggtactgccgggactttgcagaaaggcttcactacccttatcccggagagctgacaatgatgaatctcacacaaacccagggccgtccaaagcagcagagcactgagaagcatgaactgtgccccaagagtgtcccgaagagagagtacagcgtcaaagagatcctgaaactggactcccgcccttccaaagggaaggacctgcaccgctccaacatttcacccctcacttccgagaaggacgcggatgacttcaggaaagacgggagcccggagatgcccttctacccgcgcgtggtgtacccgattcgcgccccgctcccggaagactttctgaaagcacccctgggatgctacgggatggagcggccgccctacgtcactcactcccccataccgtcctccgcgacgcccagcccctcggcgcgcagcagcccggaccggagcctgcagagctgcagcccgcacagcagccccgggaacacggtgtcgccgctggcccccggcccccaggaggcccgcgactcctacgcctacttgaatgccccctacggggccgcctccgaggggctgggctccttcgccggctacgggccgcccacccacctgccgcccgccttcatcccctcctacaatgcgcactatcccaagttcctgctgcccccttacggcatgaactgcaacggcctgggcgctgccgtgggcaacatcaacagcctcaacagcttcggcctcttcccgaggctgtacccggtgtacagcagcctcttgggcgggggcggcctgcctcccccgatgctggcgcccgcctcgctccccagctcgctgccctcagagggggcgcgccgcctgctgcagcccgagcacccccgggaggtgctggtgcccgcgccgcacagtgccttctccctccccggggcggccgccagcctgaaggacaaggcctgcacccccaccagcggctccccgacggccgggaccgccgccacggccgagcacgtggtgcagcccaaagctacctcagcggccgctgcggcccccggaggcgaggaggccgtgaatctcatcaagaacaaaagaaacatgactggctacaagacgctcccctacccgctgaagaagcagaacggcaagatcaagtatgagtgcaacgtctgcgccaagactttcgggcagctctcaaatctgaaggtccacctcagagtgcacagtggagaacgacctttcaaatgccagacgtgcaacaagggcttcactcagcttgcccatctgcagaaacactacctggtacacacaggagagaagccacatgaatgccaggtttgccacaagcgatttagcagcaccagcaatctcaagacccacctgcgactccactccggagagaaaccttaccagtgcaaggtgtgtcctgccaagttcacccagtttgtgcacctgaaactccacaagcgcctacacacccgggagcggccccacaagtgtgcccactgccacaagagctacatccatttctgcagcctcagggtccacctgaaggggcactgtcctgtggccccagccactgtgctgcctctggaggacctgaccagaatcaacgaagaaattgagaagtttgacatcagcgacaatgccgaccggctggaggacatggaggacaacatcgacgtgacctctgtggtggaaaacgagattctggccgtggtcagaaaagagaaggaagaaactggcctgaaagtgtctttgcaaagaaacatgggaaatggactgctctcagggtgtagcctctatgagtcaacagagccgcccctcctgaagttgcctcacagcaacccactacctctgggacctgtaaaggtcaaacaagaaacagttgaacctatggatccttaagattttcagaaaacaaaaggtgttttgtcttgtttcttaacttacgacttggcgagtcagggtgcccgtcgcaagcgcttgtacataattccagttctgcaaagctctccctgacagcagatggtttccctcacttctctggaattaaagaaggaaccccaaagttactgaaatctcagggcataaatgaggcaaagacaatatatatacctattatatatacttacttacaccccatctatatatttgaacctgttgtattttgaatatttgtgtgaatatgtttgcatagcctccccattgctaagactattgcctagccataattattttttcaatgataatccttcataatttattatacaatttatcattcaaaaagcaataattaaaaaaagtttacaatgactggaagaattccttgtaatttgagttaagtgttatatttttgtctggtggccattctttgtagataatttctgcacatctgttttaagtatctaagatttagtaaacagataacattacttcagtcaacctccctctataataatgatttgaaaatgaggtttgagtattgccaaagttaggcagttgatgtgttcattcacaggattatattatttgaacagccttgtatcatttgaacataatttgggggtatgtttgcagaattacccagtaataggtagaagaaataaaaatcttgtatgtttctgtagagagttcagcattcctgcccccagtcacggatttaccagaaaggtggtaagaaggctttgccagtctgtctccctcttcaaaaaaacagcgtcgtcccacccccaaagtcatgtgaccatttggagtggccatgagactttagcatcccaatgtattatctgaaaatgtgaagaagacaaaatgccatgtttaaaaccactgcaaaatgtttccaaagcacaggtggttttttatgggtgaggtctgggggcacgagtccaggtgtttttgttgttggtcttttttgttatcacgtcaaaattgcacaagcatggtgctccaccaggaaggatttgagttagataggttcgggccacactttaaaaaccaaaaaccggaaaacgggtattctcgttgtcttagggtcaaagcgggtaatgaacattcctgtccccaatacatcagttgtatttgttttttctgtgcaactcagactttcctcagtatttgtgtttttacgttttatggttaatttaattgaagaagaaagggcattgcaaagttgttcaacaacagttacctcattgagtgtgtccagtagtgcaggaagtgatggcatctttaatgatttgctactcaagaggagagagcatgcgtgcgccagaaagatggaatgcgtcccattctgcctcgtcctctgctaagcccaaagatgacacgttggcattcaaggtgtagcggacaatgatgtatatttataaatctatttataccactataccatgtatatataatatatttataaccacttaaaattgtgagccaaaacatgtaaaagatccttctttcctaatggcaaaaaaaaaaaaaaaaaacaaaaaaaaaccctttctgactctctgcttaattaacaatgacctcacaactacattggtgagcttgggcacccccttggtattataagagatcctaaaatcttggtgcgaaggtggggaccgcataacaaccagggagaaagagacatacctttttaaactattaaggacaatctaagatagcttttttttttttttttgtcacattctgatgtgatcacacccaagtcccagaaatagaaatcccatcaaccactgtaacttttctctctcttcctctccttccttcttaaataactgatggatcactaatcaacctggttaattctatattcagcattttggtctgtggcaactaatgtcagcctacagaagagaacaacaaatttaggataatccagggaaacctaagagccttactgatctgttcctttaaataatgaaactgactatgatatttttttttgaacagtgtttgacaaacagtccttgattataagtaaacaaaagaatcatactctgacattgacatcaaatacattctattccccatcactcatttgttctcttttttccctctcgtgctttcttgttagtggaaatttttactgagacctgcacagtggcttgagacttcgtaattctctgtaccatctgactttccctctgaggcctcttacatgtgaatgttgagcccacaatcaacagtggttttttgttttttttttctttcttctactcaaagttaaaactgaccaaagttattggctttttactttgctagaacaaactatcatgtttacgtactggtttacattgttatttatgtgcaaattgtcaaaatgtaaattaaatataaatgttgatgcttta

SEQ ID NO: 42 - DNA, 5115 bp, Ovis aries
  source 1..5115: mol_type = other DNA
actcggccctccagtgttgcggagaggcagagcagcgcggcacctgtccgcgccgggagccgggacgcgggcgcgggcggccgagcagaagggcaccgccgcggtgcgcgccagccttcggcccggaggggacgcggagcactgcggactgtttaggcatgagcgacatttatctcagatgttggatatttgcttggaaaaacgtgtgggtacgaccttggctgcccccaagtgtagctccagtgctgtgaaggttccagggttggcagaagggaccgaagggaccatgaaaatggacatggaggacgcggatatgactctgtggacagaggctgagtttgaggagaagtgtacatacattgtgaatgaccacccctgggattctggtgtggaaggaggcactgcggttcaggcggaggcttccttaccgaggaatctgcttttcaaatgtgccacaaacaccaaagagattactggagtggtgagtaaagaatacataccaaagggaacacgttttggacctctaataggtgaaatctacaccagtgatacagttcccaagaacgccaacaggaaatatttttggcggatctattccagaggggagcttcaccacttcattgatggctttaatgaggagaagagcaactggctgcgctacgtgaacccagcacactctgcccgggagcagaacctggccgcctgtcagaacggcatgaacatctacttctacaccattaagcccatccctgccaaccaggagcttcttgtgtggtactgccgggactttgcagaaaggcttcactacccttatcccggagagctgacaatgatgaatctcacacaaacccagggccgtccaaagcagcagagcactgagaagcatgaactgtgccccaagagtgtcccgaagagagagtacagcgtcaaagagatcctgaaactggactcccgcccttccaaagggaaggacctgcaccgctccaacatttcacccctcacttccgagaaggacgcggatgacttcaggaaagacgggagcccggagatgcccttctacccgcgcgtggtgtacccgattcgcgccccgctcccggaagactttctgaaagcacccctgggatgctacgggatggagcggccgccctacgtcactcactcccccataccgtcctccgcgacgcccagcccctcggcgcgcagcagcccggaccggagcctgcagagctgcagcccgcacagcagccccgggaacacggtgtcgccgctggcccccggcccccaggaggcccgcgactcctacgcctacttgaatgccccctacggggccgcctccgaggggctgggctccttcgccggctacgggccgcccacccacctgccgcccgccttcatcccctcctacaatgcgcactatcccaagttcctgctgcccccttacggcatgaactgcaacggcctgggcgctgccgtgggcaacatcaacagcctcaacagcttcggcctcttcccgaggctgtacccggtgtacagcagcctcttgggcgggggcggcctgcctcccccgatgctggcgcccgcctcgctccccagctcgctgccctcagagggggcgcgccgcctgctgcagcccgagcacccccgggaggtgctggtgcccgcgccgcacagtgccttctccctccccggggcggccgccagcctgaaggacaaggcctgcacccccaccagcggctccccgacggccgggaccgccgccacggccgagcacgtggtgcagcccaaagctacctcagcggccgctgcggcccccggaggcgaggaggccgtgaatctcatcaagaacaaaagaaacatgactggctacaagacgctcccctacccgctgaagaagcagaacggcaagatcaagtatgagtgcaacgtctgcgccaagactttcgggcagctctcaaatctgaaggtccacctcagagtgcacagtggagaacgacctttcaaatgccagacgtgcaacaagggcttcactcagcttgcccatctgcagaaacactacctggtacacacaggagagaagccacatgaatgccaggtttgccacaagcgatttagcagcaccagcaatctcaagacccacctgcgactccactccggagagaaaccttaccagtgcaaggtgtgtcctgccaagttcacccagtttgtgcacctgaaactccacaagcgcctacacacccgggagcggccccacaagtgtgcccactgccacaagagctacatccatttctgcagcctcagggtccacctgaaggggcactgtcctgtggccccagccactgtgctgcctctggaggacctgaccagaatcaacgaagaaattgagaagtttgacatcagcgacaatgccgaccggctggaggacatggaggacaacatcgacgtgacctctgtggtggaaaacgagattctggccgtggtcagaaaagagaaggaagaaactggcctgaaagtgtctttgcaaagaaacatgggaaatggactgctctcagggtgtagcctctatgagtcaacagagccgcccctcctgaagttgcctcacagcaacccactacctctgggacctgtaaaggtcaaacaagaaacagttgaacctatggatccttaagattttcagaaaacaaaaggtgttttgtcttgtttcttaacttacgacttggcgagtcagggtgcccgtcgcaagcgcttgtacataattccagttctgcaaagctctccctgacagcagatggtttccctcacttctctggaattaaagaaggaaccccaaagttactgaaatctcagggcataaatgaggcaaagacaatatatatacctattatatatacttacttacaccccatctatatatttgaacctgttgtattttgaatatttgtgtgaatatgtttgcatagcctccccattgctaagactattgcctagccataattattttttcaatgataatccttcataatttattatacaatttatcattcaaaaagcaataattaaaaaaagtttacaatgactggaagaattccttgtaatttgagttaagtgttatatttttgtctggtggccattctttgtagataatttctgcacatctgttttaagtatctaagatttagtaaacagataacattacttcagtcaacctccctctataataatgatttgaaaatgaggtttgagtattgccaaagttaggcagttgatgtgttcattcacaggattatattatttgaacagccttgtatcatttgaacataatttgggggtatgtttgcagaattacccagtaataggtagaagaaataaaaatcttgtatgtttctgtagagagttcagcattcctgcccccagtcacggatttaccagaaaggtggtaagaaggctttgccagtctgtctccctcttcaaaaaaacagcgtcgtcccacccccaaagtcatgtgaccatttggagtggccatgagactttagcatcccaatgtattatctgaaaatgtgaagaagacaaaatgccatgtttaaaaccactgcaaaatgtttccaaagcacaggtggttttttatgggtgaggtctgggggcacgagtccaggtgtttttgttgttggtcttttttgttatcacgtcaaaattgcacaagcatggtgctccaccaggaaggatttgagttagataggttcgggccacactttaaaaaccaaaaaccggaaaacgggtattctcgttgtcttagggtcaaagcgggtaatgaacattcctgtccccaatacatcagttgtatttgttttttctgtgcaactcagactttcctcagtatttgtgtttttacgttttatggttaatttaattgaagaagaaagggcattgcaaagttgttcaacaacagttacctcattgagtgtgtccagtagtgcaggaagtgatggcatctttaatgatttgctactcaagaggagagagcatgcgtgcgccagaaagatggaatgcgtcccattctgcctcgtcctctgctaagcccaaagatgacacgttggcattcaaggtgtagcggacaatgatgtatatttataaatctatttataccactataccatgtatatataatatatttataaccacttaaaattgtgagccaaaacatgtaaaagatccttctttcctaatggcaaaaaaaaaaaaaaaaaacaaaaaaaaaccctttctgactctctgcttaattaacaatgacctcacaactacattggtgagcttgggcacccccttggtattataagagatcctaaaatcttggtgcgaaggtggggaccgcataacaaccagggagaaagagacatacctttttaaactattaaggacaatctaagatagcttttttttttttttttgtcacattctgatgtgatcacacccaagtcccagaaatagaaatcccatcaaccactgtaacttttctctctcttcctctccttccttcttaaataactgatggatcactaatcaacctggttaattctatattcagcattttggtctgtggcaactaatgtcagcctacagaagagaacaacaaatttaggataatccagggaaacctaagagccttactgatctgttcctttaaataatgaaactgactatgatatttttttttgaacagtgtttgacaaacagtccttgattataagtaaacaaaagaatcatactctgacattgacatcaaatacattctattccccatcactcatttgttctcttttttccctctcgtgctttcttgttagtggaaatttttactgagacctgcacagtggcttgagacttcgtaattctctgtaccatctgactttccctctgaggcctcttacatgtgaatgttgagcccacaatcaacagtggttttttgttttttttttctttcttctactcaaagttaaaactgaccaaagttattggctttttactttgctagaacaaactatcatgtttacgtactggtttacattgttatttatgtgcaaattgtcaaaatgtaaattaaatataaatgttgatgcttta

SEQ ID NO: 43 - DNA, 1716 bp, Bos taurus
  source 1..1716: mol_type = other DNA
atggccctgcggccgccgggggaggccggcccccaggacaagggatgctaccctcccgactggctccagggcagcccccaacacctggccgcctactacacgcctttcccgccctacggccactacgggaacaccctgaccgcggcagaggagggcttccagcctttccggcccctggaagccctggcagcgtccccggctctgccccccttcggcttccggaccgcgcctcccttgctaagccagggcctggccctcccgagggagccgctatgcgacctgccctggtacagcaagctgccgacctggtaccctgttccccacctccccagggaggtgccgcacttccagaacagcagtcgtgagtttaccggcgccgccagcgaggacttggcacacaccgccggccggagcgacggtggccagtgttggggacctgagattttcgttccgccgccccccgtggatgcttcgttgttacctgagaggccgaagccctcgcagctgttatcggggtcccccagcgagccctctgaggatggccccaaactcgtgaaccgaaaagggaaggcttctcgccgctaccacttcacccaagaagacctgcacttggttctgtacggggtcatccccagcccgcagcactcagcccgcctgcagcacgcgatctcgggcctcctgatccccaccgatagcgccggggctgattctcttcctcagactctggataacgactcccttcagctaccagaaggtctctgcctccagcagacgaagtttggtgaagtcgcacattttggggtgttctgcagtagtcttgtggccaaaggagtcaggtttgggccctttcagggtaaagtggtcaacaccagcgaagtcaagacacacggagacaattccctgatgtgggagatctttgaagatggtcagttgagccactttattgatggaaaaggaggtgcagggaactggatgtcctatgtcaactgtgcccgtttcccgtcggagcagaacctggtggcagtacaatgtcaagggcagatcttctacgagagctgcaaggagatctaccagaaccaggagctcctcgtgtggtacggagattgctatgagaagtttctggacattcccgtgagcctgcaggttctcaagcaggggaagcaggcatctgggtcctctgaagagtctgcggaagggtacaggtgtgaaagatgtggcaaagtatttacctacaaatattacagagacaagcatctaaagtacacgccttgtgtggacaagggtgacaggaaatttccctgttctttatgcaaacgatcctttgagaagagagaccggctccggatccacatccttcacgttcatgagaagcatcggcctcacaagtgttctacatgcgggaaatgtttctctcagtcttccagtctaaacaaacacatgagagtccactctggagacagaccctaccactgtgtgtattgtactaagaaattcactgcctccagtatactccgcacgcatatcaggcagcactcgggggagaagcccttcaaatgcaagtactgtggtaaatcttttgcatcgcacgctgctcacgacagccatgtccggcgctcgcacagggaggatgacggctctccctgtaacacctgtgggaagaccttctcagatcaagaagccttccactcccacatgaagtctcatgaagactgctag

SEQ ID NO: 44 - DNA, 1915 bp, Sus scrofa
  source 1..1915: mol_type = other DNA
tcgcggtggcccaggatggctctgcggctgccaggtgagaccggtccccaggacaaggagtgctactcgcccgacaggcttcagagcagcccccaacacctggccgcctactacacccctttcccgcactatggccattacgggaacaccctcaccacggcggaggagggtttccaacctttccggcagctggaggccctgtcggcgtccccggctctgccccccttcgccttccggacagcgcctcccttgctgagccccgggctggcgctgccgagggagccgctctatgatgtgccctggtacagcaagctgccgacgtggtacccgatcccccacctgcccagggaggtgccacacttcctgaacagcccgcgcgagttcacgggcgcccccgccgaaaacttgggccaggtcggcggccggagcgatggtggccagtgttggggacctgaagttttccttccgccgcccccagtggatgcttctctgctacccgagaggccgaagccctcccagttgttacctggctcccccggcgagcgctctgaggatggccccaaactcgtgaaccaagagagaaaggcatcgccccgttaccacttcacccaggaagacctgcacttagttctgtacggggtcattcccagccctcagcacccagcccgcttgcaccacgcgatgtcgggcctcctggtccccaccaacagctctgggtctgattctcttcctcagactctggataacagctcccttcagctcccggaaggtctctgcctgcggcagacgatgtttggtgaggtcgcacattttggtgtgttctgcaatagcctcgtggccaaaggagtcaggtttggaccctttcagggtaaagtggtcaatgccagtgaagtcaagacccacggagacaattctctgatgtgggagatttttgaagatggtcatttgagccactttattgatggaaaaggaggtgctgggaactggatgtcctatgtcaactgtgcccgtttccccacggagcagaacctagttgcggtgcaatgtcaagggcagatattctatgaaagctgcaaggagatccaccagaaccaagagctcctggtgtggtatggagattgctacgaaaagtttttggacattcctgtgagcctgcaggtcataaagcaggggaagcaggcatctgggccctcagaagagtctgcagaagggtacagatgtgaaagatgtggaaaagtatttacctacaaatattacagagacaagcatctcaagtacaccccctgtgtggacaagggtgacaggaaatttccctgtaatttgtgcagacgatcctttgagaagagagaccggctccggatccatatccttcacgtccatgagaagcaccgaccccacaagtgttctacgtgtgggaaatgtttctctcaatcttccagcctaaacaagcacatgagagtccactctggagacagaccctaccagtgtgtgtattgtacaaagaagttcactgcctctagtatactccgcacccacatcaggcagcactccggggagaagcccttcaaatgcaagtactgtggtaaatcttttgcgtcccacgccgcgcacgacagccatgtccggcgttcacacaaggaggatgacagctgttcctgtagcatctgtggcaagaccctccccaatcaagacgctttctatgcccacatgaagtttcatgaagactactaactagcctttagagattttgaggatatttcttgtttatgggtgtgtcttgtggggaaaagtgatgcttatattgagatgagacacagagtagtggaactgaccaattagcagaatttgccagagttcttggttgtggtattaacatcctttgcagaaatgttttttgttttttgtttttgtcttt

SEQ ID NO: 45 - DNA, 1710 bp, Ovis aries
  source 1..1710: mol_type = other DNA
atggccctgcggccgccgggggaggccggaccccaggacaagggatgctacccggccgactggctccagagcagcccccaacacctggccgcctactacacgcctttcccgccctacggccactacgggaacaccctgaccgcggcggaggagggcttccagcctttccggcctctggaagccttgggagcgtccccggctctgccccccttcggcttccggaccgcgcctcccttgctgagcccgggcctggccctgccgagggagccgctgtgcgacctgccctggtacagcaagctgcagacctggtaccccgttccccacctccccagggaggtgccacacttccaaaacagcagtcgcgagtttaccggcgccgccagcgaggactcggcacacaccgccggacggagcgacggtggccagtgttggggacctgaaattttcgttccgccgccccccgtggatgcttcgttgttacctgagaggccgaagccctcgcagctgttatcggggtcccctagagagccctctgaggatggcgccaaactcgtgaaccaagaggggaaggcttctcgccgctaccacttcacccaggaagacctgcacttggttctgtacggggtcatccccagcccgcagcactcagcccgcctgcaccacgcgatctcgggcctcctgatccccaccgacagcgccggagctgattctcttcctcagactctggataacgactcccttcagctaccagaaggtctctgcctccagcagacgaagtttggtgaagtcgcacattttggggtgttctgcagtagtcttgtggccaaaggagtcaggtttgggccctttcagggtaaagtggtcaacaccagcgaagtcaagacacacggagacaattccctcatgtgggagatctttgaagatggtcagttgagccactttattgacggaaaaggaggtgcagggaactggatgtcctatgtcaactgtgcccgtttcccgtcggagcagaacctggtggcaatacaatgtcaaggacagatcttctacgagagctgcaaggagatctaccagaaccaggagctcctcgtgtggtacggagattgctatgagaaatttctggacattcccgtgagcctcctcaagcaggggaagcaggcgtctgggtcctctgaagagtctgcggaagggtacaggtgtgaaagatgtggcaaggtgttcacctacaaatactacagggacaagcatctgaagtacacgccttgtgtggacaagggtgacaggaaatttccctgttctttatgcaaacgatcctttgagaagagagaccggctccggatccacatccttcacgttcatgagaagcataggcctcacaagtgttctacatgcgggaagtgtttctctcaatcttccagtctaaacaaacacatgagagtccactctggagacagaccctaccactgtgtgtattgtactaagaaattcactgcctccagtatactccgcacgcatatcaggcagcactcgggggagaagcccttcaaatgcaagtactgtggtaaatcttttgcatcgcacgctgctcacgacagccatgtccggcgctcgcacagggaggatgatggctctccctgtaacacctgtggaaaaaccttctcggatcaagaagctttccactcccacatgaagtctcatgaagactgctag

SEQ ID NO: 46 - DNA, 5144 bp, Bos taurus
  source 1..5144: mol_type = other DNA
cttggcgctcgcggctctgggggctcggctttgccgcgctcccggcactcgggcgagagccggaacgtggaacagagctccggtcccagcgcagccaccgcgatgagaggcgctcgcggcgcctgggatttcctcttcgttctgctgctcctgctcctcgtccagacaggctcttctcagccatctgtgagtccaggggaactgtctctaccatctatccacccagcaaaatcagagttaattgtcagcgttggcgacgagattaggctgttatgcaccgatccaggatttgtcaagtggacttttgagatcctgggtcaactgagtgagaaaacaaacccggaatggatcaccgagaaagcagaggccacaaatacaggcaattacacgtgcaccaataaaggcggcttgagcagttccatttatgtgtttgttagagaccccgagaagcttttcctgattgaccttcccttgtacgggaaagaagaaaacgacacgctggttcgctgtcccctgacagaccccgaggtgaccaattactctcttacggggtgtgaggggaaacctctccccaaggatttgacatttgtggccgaccccaaggcaggtatcacaatcagaaatgtgaagcgcgagtaccatcggctctgtctgcactgctcagcgaatcagaggggcaagtccatgctgtcgaagaaattcactctgaaagtgcgggcagccatcaaagctgtgccagttgtgtctgtgtccaaaaccagctatcttctcagggaaggagaggaatttgcagtgacatgcttgattaaagacgtgtctagttccgtggactctatgtggataaaggaaaacagccagcagactaaagcacagatgaagaagaatagctggcatcagggtgacttcagttatctccgtcaggaaaggttgactatcagctcagcaagagtgaatgattccggtgtgttcatgtgttacgccaataacacttttggatcagcaaatgtcacaacaaccttagaagtagtagataaaggattcattaatatcttccctatgatgaacacaacagtatttgtaaatgatggagagaatgtggatctggttgttgaatatgaggcatatcccaaacctgtacaccgacaatggatatatatgaacagaacctccactgataagtgggacgattatcctaagtctgaaaatgaaagtaatatcagatacgtaaatgaacttcatctaaccagattaaaagggactgaaggaggcacttacacatttcacgtgtccaattctgatgtcaattcttccgtgacatttaatgtttacgtgaacacaaaaccagaaatcctgacgcatgacaggctggtgaatggcatgctacagtgcgtggccgcagggttcccggagccaaccatcgattggtacttttgtccaggaactgagcagaggtgttctgttcccgttgggccagtggatgtacagatccaaaactcatctgtctcaccatttggaaaactagtggtttatagcaccattgatgacagcacattcaaacacaatgggacggtggagtgcagggcttataacgatgtgggcaagagttctgcctcttttaactttgcatttaaaggtaacagcaaagaacaaatccatgctcacaccctgttcacgccgttgctgattggttttgtgatcgcagctggtttaatgtgtatcttcgtgatgattcttacatacaaatatttgcagaaacccatgtatgaagtacagtggaaagttgtcgaggagataaatggaaacaattatgtttacatagacccaacacaacttccttatgatcacaaatgggagtttcccaggaacaggctgagttttgggaaaaccctgggtgctggcgccttcgggaaagttgttgaggccaccgcttatggcttaattaaatcagatgcagccatgactgttgctgtcaagatgctcaaaccaagcgcccatttaaccgaacgagaagccctaatgtctgaactcaaagtcttgagttacctcggtaatcatatgaatattgtgaatcttctgggagcgtgcaccattggagggcccaccctggtcattacagaatattgttgctatggtgatcttctgaattttttgagaagaaaacgtgattcatttatttgctcaaagcaggaagatcacgccgaagtggcgctttataagaaccttcttcattcaaaggagtcttcctgcaatgatagtactaatgagtacatggacatgaaacctggagtttcttatgttgtaccaaccaaggcagacaagaggagatctgcaagaataggttcatacatagaaagagacgtgactcctgctatcatggaagatgatgagctggccctggacctggaggacttgctgagcttttcttaccaggtggcaaaaggcatggcgttccttgcctcaaagaattgtattcatagagacttggcagccagaaatatcctccttactcatggtcgaatcacaaagatttgtgattttggtctagccagagacatcaagaatgattctaattatgtggtcaaaggaaacgctcgactccctgtgaagtggatggcaccagagagtattttcaactgtgtatacacatttgaaagtgatgtctggtcctatgggatttttctgtgggagctgttctctttaggaagcagcccctaccctggaatgccagtcgattctaagttctacaagatgatcaaggaaggtttccgaatgctcagccccgagcatgcacctgcggaaatgtatgacatcatgaagacctgctgggatgctgatcccttgaaaaggccaacatttaagcagattgtgcagctgattgagaagcagatctcagagagcaccaatcatatttattccaacttagcaaactgcagcccccaccgggagaaccccgccgtggaccattctgtgcgcatcaactctgtgggcagcagcgcctcctccacgcagcctctgcttgtccacgaagatgtctgaagcagtctgcatctgggggtctcctgacaaccccgaaccccctctcttttggttttcacaatggttcctttgttttccttccactcgaatcctactccagggtagtggacaccctgatgtaatcctgtctttatgagcaccctttagtggctgatgatttttgtcatcagctaccatcaagctgcctatgttcctaatagcacacaagcccctgctaacaaggaaacttcagacttggagaaagggagggtgggatggactagacacactgagtccttcccaaggtttctccaatttctatctgaaaaatgtagttgatagttttgagtacatagcagtagtcaccttcattcctcataatcatccatgatggtatgatgatgtagcaagactagaagctgaaaccttatcccttgatgcggaaaatagaatgttattcaaaggccagaagcctatgaatatctgggctcacgaaatctagtatttcatgctgggagtaagacataggccatggaaaaatgttctccaggcctgaataaagactgctgggcatgagccttcgtgcttctgacctggtttctaaatcaaattcactaatagtagctgaattggagagatggcctcccagcccacattttgtatatactcatctataaattgtatgcattcacatatttgaggggggaaaacccacaaggtgtagtttctgaatacaattctggcttgagtctgctgtgtataggaataactgatgagccagaacaagtttgaaggaaacagtgctttaaaaaaaaaaaattattgccaagcacagtcttaacaaaaatctccttttgtagccgatgaacttgctctagagattgtccagaacaagccgaccagcttcagaatgtcatcgtgttcagtggatttgatgccgtttgaaaaagtgatttattgcatgactctggcaggagtgacaaccagtgctatcttagtttggattcttctgtagccaaaaataaactttagattcagcctccatcacaggcatgtcctggacacctggccagtatctatataagtgtgtatgtgtgtgtgtgtatgtatgcttgtagacaaaatattttgggagtagtcttgccttgagcccagaagggtccttcagcacccaagaagtagcctggctttcagcagcagcccagcttttgtttcacttcaccttgctgtatagggaagcttaccaaaagcctacatagaggagtgcttaagtccatatttatttgcaatccacctgcacttaaggcactctgttacttatactcaacatactgtacctgttccttagaccttattaatgctactgtcctactgaaacatatttcagttctatctaccctttaggctgtagcctggatattattcttagtgttacctgtttgtttgtttgttttttttttttgcctctacccaattataagaggtccatgggtacatggcagggtttatgttgtctcgcaggattcaggtatgctgccctcacggttttcccctcctaggtgtctttgctttcatttagagaactgtggccatttatctgaaagtaaccatttgcactggagttctatgctctcgcacctttccaaaggtaacaggttggggcgttttgttgctaatgtaagacgtttttgctgtttgccacactttgtctgaaaattttctttacgtttctattgactttggttatagtaagaaaagtggttgttaattgtagatgtctaggtacttcaggggcacttcactgagagttttgtcttggatattcttgaaagtttatatttttataattttttttttttttacatctgatgtttctttgcagtagctttatgtttgaaattattttgtggcttttttttttgtaaatattgaaatgtagcaataatgtcttttgaatattcccaatcccatgagtccttgaaaatattttttatatatacagtaactttatgtgtaaatatgtaagcggtgcaggtttaaaggttgtttatgttctatgtgtttttttcctgatacgttgtccaactgttcacagttctaaagaattctaataaaagtgtaaatataaa

SEQ ID NO: 47 - DNA, 3719 bp, Sus scrofa
  source 1..3719: mol_type = other DNA
ggagctccggtcccagagcagccaccgcgatgagaggcgctcgccgcgcctgggattttctcttcgtcctgcagctcttgcttcgcgtccagacaggctcttctcagccatctgtgagtccagaggaactgtctccaccatccatccatccagcaaaatcagagttaatcgtcagtgctggcgatgagattaggctgttctgcaccgatccaggatctgtcaaatggacttttgagaccctgggtcagctgagtgagaatacwcacgcagagtggatcgtggagaaagcagaggccatgaatacaggcaattatacatgcaccaatgaaggcggtttaagcagttccatttatgtgtttgttagagatcctgagaagcttttcctcgtcgaccctcccttgtatgggaaggaggacaatgacgcgctggtccgmtgtcctctgacggacccagaggtgaccaattactccctcacgggctgcgaggggaaaccccttcccaaggatttgaccttcgttgcagaccccaaggccggcatcaccatcaaaaatgtgaagcgcgagtatcatcggctgtgtctacactgctccgccaaccaggggggcaagtccgtgctgtcgaagaaattcaccctgaaagtgagggcagccatcagagctgtacctgttgtggctgtgtccaaagcaagctaccttctcagggaaggggaggaatttgccgtgatgtgcttgatcaaagacgtgtctagttcygtggactccatgtggatcagggagaacagccagactaaagcacaggtgaagaggaatagctggcatcagggtgacttcaattttctgcggcaggaaaagctgacgatcagctcagcaagagttaatgattctggcgtgttcatgtgttacgccaataatacttttggatctgcaaatgtcacaaccaccttagaagtagtagataaaggattcattaatatcttccctatgatgaataccactgtgtttgtaaacgatggagaggatgtggatctaattgttgagtacgaggcgtaccccaaacctgaacaccgacagtggatatatatgaaccgcactgccactgataagtgggaggattatcccaagtctgagaatgaaagtaacatcagatatgtaagtgaacttcacttgaccagattaaaagggaccgaaggaggcacttacacatttctcgtgtccaatgctgatgtcaattcttctgtgacatttaatgtttacgtgaacacaaaaccagaaatcctgactcatgacaggctcatgaacggcatgctccagtgtgtggcggcaggcttcccagagcccaccatcgattggtatttctgtccaggcaccgagcagagatgttccgttcccgttgggccagtggacgtgcagatccaaaactcatctgtatcaccgtttggaaaactagtgattcacagctccattgattacagtgcattcaaacacaacggcacrgtggagtgcagggcttacaacgatgtgggcaagagttctgccttttttaactttgcatttaaagaacaaatccatgcccacaccctcttcacgcctttgctgattggttttgtgatcgcagcgggtatgatgtgtatcatcgtgatgattctcacctataaatatctacagaagcccatgtatgaagtacagtggaaggttgtcgaggagataaatggaaacaattatgtctacatagacccaacgcaacttccttatgatcacaaatgggaatttcccaggaacaggctgagttttggcaaaaccttgggtgctggcgccttcgggaaagtcgttgaggccactgcatacggcttaattaagtcagatgcggccatgaccgttgccgtgaagatgctyaaaccaagtgcccatttaacggaacgagaagccctratgtctgaactcaaagtcttaagttacctcggtaatcacatgaatattgtgaatcttctcggcgcctgcaccattggagggcccaccctggtcattacagaatattgttgctatggtgatctcctgaattttttgagacggaaacgtgattcgtttatttgctcaaagcaggaagatcacgcagaagcggcgctttataagaaccttctgcattcaaaggagtcttcctgcagtgacagtactaacgagtacatggacatgaaacccggagtgtcttatgtggtaccaaccaaggcagayaaaaggagatctgcgagaataggctcatacatagaacgagatgtgactcctgccatcatggaagatgatgagttggccctagacctggaggacttgctcagcttttcttaccaagtggcaaagggcatggccttcctcgcctcgaagaattgtattcacagagacttggcggccagaaatatcctccttactcatggtcgaatcacaaagatttgtgattttggtctagccagagacatcaagaatgattctaattacgtggtcaaaggaaacgctcggctacccgtgaagtggatggcacccgagagcattttcaactgtgtctacacatttgaaagcgatgtctggtcctatgggatttttctgtgggagctcttctctttagggagcagcccctacccyggaatgccagttgattctaaattctacaagatgatcaaggagggtttccgaatgctcagccctgagcatgcacctgcggaaatgtatgacatcatgaagacttgctgggatgcggatcccctcaaaagaccaacgtttaagcagatygtgcagctgattgagaagcagatttcggagagcaccaatcacatttattccaacttagcgaactgcagcccccaccgggagaaccccgcggtggatcattctgtgcggatcaactccgtgggcagcagtgcctcctccacgcagccgctgcttgtccacgaagacgtctgaagcagaatgggtgtccggggtggggggtgggggggctcccccacagcaccgacctactgccattctttttggttttcataatggttattttgtttcccttcaacttgcatcctactccagggtagtggatgctccgctgtaatcctctttacgagcacactttagtggccaatgatttttgtcatcagctgccattgagctgtatatgttcccaatagcacgctagcccccattaacggagagcattcagacttagggaagaggagggtaggacgggctggacaccccaggtccttgacaagtcttctccagtttctgtccaataagtgctgtaatggtttatttgagcacctggctgtcgtcacctccggtccttgtcatcatctgtaacaatatgatgatgatgatgccagaacctaatcccttgatgtggaaaataggatgttaatcaaacaaagggcagaaagaagcctgtgactatctgggctcgagaagtcaagtatttcatgctgggagtaagacgtaagccatggaaaaatgctctccgggcatgaataaggctgctggccatgagcctttttactcctgacctggtttctaagtagtttgttattagggagctggatcggagggaaggcttctgcctgcattttgtatatactcatctataaattgttcatgttcacatatttgaggggggaaaacccgcaaggtgtagtttctggatacaatcctggctcgagtctgctgcgtgtagaaatagctgaagag

SEQ ID NO: 48 - DNA, 3719 bp, Sus scrofa; identical to SEQ ID NO: 47
  source 1..3719: mol_type = other DNA

SEQ ID NO: 49 - DNA, 3789 bp, Ovis aries
  source 1..3789: mol_type = other DNA
ccgcgctcccggcactcgggctagagccggaacgtggaacagagctccggtcccagcgcagccaccgcgatgagaggcgctcgcggcgcctgggatttcctcttcgttctgctgctcctgctcctcgtccagacaggctcttctcaaccatctgtgagtccaggggaactgtctctaccatctatccatccagcaaaatcagagttaattgtcagtgttggcgacgagattaggctgtcatgcactgatccaggatttgtcaagtggacttttgagatcctgggtcaactgagtgagaagacaaacccggaatggatcaccgagaaagcagaggccacaaatacaggcaattacacgtgcaccaataaaggcggcttgagcagttccatttatgtgtttgttagagaccctgagaagcttttcctgattgaccttcccttgtacgggaaagaagaaaacgacacgctggttcgctgtcccctgacagaccccgaggtgaccaattactctctcacggggtgtgaggggaaacctctccccaaggatctgacatttgtggctgaccccaaggcaggcatcacaatcagaaatgtgaagcgcgagtaccatcggctctgtctgcactgctcagcgaatcagaagggcaagtccatactgtcgaagaaattcaccctgaaagtacgggcagccatcaaagctgtgccggttgtgtctgtgtccaaaaccagctatcttctcagggaaggagaggaatttgcagtgacatgcttgattaaagacgtgtctagttccgtggactctatgtggataaaggaaaacagccagcagagtaaagcacagacgaagaagaatagctggcatcagggtgacttcagttatctccgtcaggaaaggctgactatcagctcagcaagagtgaatgattccggtgtgttcatgtgttacgccaataatacttttggatcagcaaatgtcacaacaaccttagaagtagtagataaaggattcattaatatcttccctatgatgaacacaacagtgtttgtaaatgatggagagaatgtggatctggttgttgagtatgaggcatatcccaaacctgaacaccgacagtggatatatatgaacaggacctccactgataagtgggacgattatcccaagtctgagaatgaaagtaacatcagatacgtaaatgaacttcatctaaccagattaaaagggactgaaggaggcacttacacatttcacgtgtccaattctgatgtcaattcttccgtgacatttaatgtttacgtgaacacaaaaccagaaattctgacgcatgacaggctggtgaatggcatgctacagtgcgtggccgcagggttcccggagccaaccattgattggtacttttgtccaggaaccgagcagaggtgttctgttcctgttgggccagtggatgtacagatccaaaactcatctgtcccaccatttggaaaactagtggtttatagcaccattgatgacagcacattcaaacacaatgggacggtggaatgcagggcttacaacgatgtgggcaagagttctgcctcttttaactttgcatttaaagaacaaatccatgctcacaccctgttcacgccgttgctgattggttttgtgatcgcagctggtttaatgtgtatcttcgtgatgattcttacatacaaatatttgcagaagcccatgtatgaagtacagtggaaagttgtcgaggagataaatgggaacaattatgtttacatagacccaacacaacttccttatgatcacaaatgggagtttcccaggaacaggctgagttttgggaaaaccttgggtgctggcgcctttgggaaagtcgttgaggccactgcttacggcttaattaaatcggatgcagccatgactgttgctgtcaagatgctcaaaccaagcgcccatttaacagaacgagaagccctaatgtctgaactcaaagtcttgagttacctcggtaatcatatgaatattgtgaatcttctgggagcatgcaccattggagggcccaccctggccattacagaatattgttgctatggtgatcttctgaattttttgagaagaaaacgtgattcatttatttgctcaaagcaggaagatcatgccgaagtggcactttataagaaccttcttcattcaaaggagtcttcctgcaatgatagtactaatgagtacatggacatgaaacctggagtttcttatgttgtaccaaccaaggcggcagacaagaggagatctgcaagaataggttcatacatagaaagagacgtgactcctgctatcatggaagatgatgagctggccctggacctggaggacttgctgagcttttcttaccaggtggcaaagggcatggcgttccttgcctcaaagaattgtattcatagagacttggcagccagaaatatcctccttactcatggtcgaatcacaaagatttgtgattttggtctagccagagacaccaagaatgattctaattatgtggtcaaaggaaacgctcgactccctgtgaagtggatggcaccggagagtattttcaactgtgtgtacacatttgaaagtgatgtctggtcctatgggatttttctgtgggagctgttccctttaggaagcagcccctaccctggaatgccagtcgattctaagttctacaagatgatcaaggaaggtttccgaatgctcagccccgagcatgcacctgcggaaatgtatgacatcatgaagacctgctgggatgcagatcccttgaaaaggccaacatttaagcagattgtgcagctgattgagaagcagatctcagagagcaccaatcatatttattccaacttagcaaactgcagcccccaccgggagaaccccgcggtggaccattctgtgcgcatcaactctgtgggcagcagcgcctcctccacgcagcctctgcttgtccacgaagatgtctgaagcagagtctgcatccaggggtctcctgaacaaccccggcccccctctcttttggtttttacaatggttcctttgttttccttcgactcgaatcctactcaagggtagtggacaccctgtctttatgagcaccctttagtggccgatgatttttgtcatcagctaccatcgagctgcctatgttcctaatagcacactagcccctgctaacgaggaaacttcagacttggagaaagggtgggtgggatggactggacacactgagtccttcccaaggtttctccaacttctatccgaaaaatgtagttgatagttttgagtacatagcagtagtcaccttcattcctcataatcacccatgatggtatgatgatgcagcaaggctagaagctgaaaccttatcctttgatgtgggaaatagaatgttattcaaaggccagaagcctatgaatatctgggctcaagaaatctagtatttcatgctgggagtaagacataggccatggaaaaatgctctctgctcctgaataaagactgctgggcatgagcctttgtgcttctgacctggtttctaagtcaaattcactaatagtagctgaattggagagatggcctcccagcccacattttgtatatactcatctataaattgtacacattcacatattgaggggggggaacccacaaggtgtagtttctgaatacaattctggcttgagtctgctgtgtataggaataactgatgagccagaacaagtttgaaggaaacagtgctttaaaaaaaaaaaaaaaaaa

SEQ ID NO: 50 - DNA, 1816 bp, Bos taurus
  source 1..1816: mol_type = other DNA
ggggaaatgtgttccttgctaattcttccaagccatggcgcttcccaacaaattcttcctttggttttgctgctttgcctggctctgttttcctattagccttgattctcagccttctaggggagaagctcagattgtagctaggactgcgttggaatctgaggctgagacttggtccttgctgaagcatctagatgggagacacagacctggtctcctttcccctctcttaaacgttctgtatgatgggcacagggaaccccccaggcttcagccagatgacagagctttgagctacatgaagaggctctataaagcatatgctaccaaggaggggacccctaaatccaacagaagccacctctacaacactgttcggctcttcaccccctgtgctcagcacaagcaggctcctggggaccaggcagcaggaacccttccatcagtggatctgctgtttaacctggatcgtgttactgttgtggaacatttattcaagtcagtcttgctatatactttcaacaactccatttcttttccctttcctgttaaatgtatatgcaacctggtgataaaagagccagagttttctagcaagactctccctagagctccatactcatttacctttaactcacagtttgaatttagaaagaaatacaaatggattgagattgatgtgacagctcctcttgagcctctggtggcctcccacaagaggaatattcacatgtctgtaaattttacatgtgtgaaagaccagctgcagcatccttcagcacgggacagcctgtttaacatgactcttctcttagcgccctcactgcttctatatctgaacgacacaagtgctcaggcttttcacaggtggcattccctccaccctaaaaggaagccttcacaggatcctgaccagaagagagggctgtctgcctgtcccatgggagaagaagctgctgagggtgtaagattgtcccgtcaccggagagaccaggagagtgtcagctctgaattgaagaagcctctggttccagcttcattcaatctgagtgaatacttcaaacagtttctttttccccagaatgaatgtgagctccatgactttagacttagctttagtcaactgaagtgggacaactggattgtggccccacacaaatacaaccctcgatactgtaaaggggactgtcccagggcggtcggacatcggtatggctctccagttcacaccatggtgatgaacatcatccatgagaaacttgactcctcagtgccaagaccatcctgtgtacctgccaagtatagtcctttgagtgttttggccatcgagcctgatggctcaattgcttataaagaatatgaagatatgatagccactaagtgtacctgtcgttaacacagactcctgtcaagtaaaaccgtgagtgtcctggccagtgtaaatgccgtgcacctgtctatgcctttgggaggaagtttcatgtgtcaaatctctaactgtactacagtgtgtgttgtgtaaggaggagcctgtatagattagcgtattctgtggcatctatcaatgtaaagggataacagctgccttcctcatggccaaatgtttaaatgaaatgtatttcttttggagaactttaaattttttcccaagtaattcttttttctttttataggagtcttctttttgatgtaagaaaattaaaaaacaattagcataaatcttgggtggaattgcagttctagacaattcatagtcttatttaatggaggaataaattcctgtgagtggcaaaaaaaaaaaaaaaaaaaaaaaaaa

SEQ ID NO: 51 - DNA, 2754 bp, Bos taurus
  source 1..2754: mol_type = other DNA
gtgttccttgctaattcttccaagccatggcgcttcccaacaaattcttcctttggttttgctgctttgcctggctctgttttcctattagccttgattctcagccttctaggggagaagctcagattgtagctaggactgcgttggaatctgaggctgagacttggtccttgctgaagcatctagatgggagacacagacctggtctcctttcccctctcttaaacgttctgtatgatgggcacagggaaccccccaggcttcagccagatgacagagctttgagctacatgaagaggctctataaagcatatgctaccaaggaggggacccctaaatccaacagaagccacctctacaacactgttcggctcttcaccccctgtgctcagcacaagcaggctcctggggaccaggcagcaggtgtgtgggagcagattggttaatgggtggagggaagaagaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggcattactgttggattgtttttcttctcaggaacccttccatcagtggatctgctgtttaacctggatcgtgttactgttgtggaacatttattcaagtcagtcttgctatatactttcaacaactccatttcttttccctttcctgttaaatgtatatgcaacctggtgataaaagagccagagttttctagcaagactctccctagagctccatactcatttacctttaactcacagtttgaatttagaaagaaatacaaatggattgagattgatgtgacagctcctcttgagcctctggtggcctcccacaagaggaatattcacatgtctgtaaattttacatgtgtgaaagaccagctgcagcatccttcagcacgggacagcctgtttaacatgactcttctcttagcgccctcactgcttctatatctgaacgacacaagtgctcaggcttttcacaggtggcattccctccaccctaaaaggaagccttcacaggatcctgaccagaagagagggctgtctgcctgtcccatgggagaagaagctgctgagggtgtaagattgtcccgtcaccggagagaccaggagagtgtcagctctgaattgaagaagcctctggttccagcttcattcaatctgagtgaatacttcaaacagtttctttttccccagaatgaatgtgagctccatgactttagacttagctttagtcaactgaagtgggacaactggattgtggccccacacaaatacaaccctcgatactgtaaaggggactgtcccagggcggtcggacatcggtatggctctccagttcacaccatggtgatgaacatcatccatgagaaacttgactcctcagtgccaagaccatcctgtgtacctgccaagtatagtcctttgagtgttttggccatcgagcctgatggctcaattgcttataaagaatatgaagatatgatagccactaagtgtacctgtcgttaacacagactcctgtcaagtaaaaccgtgagtgtcctggccagtgtaaatgccgtgcacctgtctatgcctttgggaggaagtttcatgtgtcaaatctctaactgtactacagtgtgtgttgtgtaaggaggagcctgtatagattagcgtattctgtggcatctatcaatgtaaagggataacagctgccttcctcatggccaaatgtttaaatga

SEQ ID NO: 52 - DNA, 1335 bp, Sus scrofa
  source 1..1335: mol_type = other DNA
atggcgcttcccagaaaattcttcctttgtttttgctgctttgccttgttctgttttcctgttagctgtggttctcaggcttctagaagagaagctcactttgcaactagtgctgtgttggaatctgaggctgagccttggtccttgctgcggcctccagatgagagacacagatctggcctcccttcccccctcttcaatgttctgtatgatgggcatggggggttgcccaggctgcagccagactccagagctttgcgctacatgaagaggctgtataaaacatatgctaccaaggaggggatccccaaagccaacagaagtcacctctacaacactgtccggctcttcacaccctgtgcccagcataagcaggctcctggggaccaggtgacaggaacccttccatcaggggacctgcggtttaacctggatcgtgttactgctgttgaacatttactcaagtcagtcttgttatacacgtggaacaactccatttcttttccctcccctgttaaatgtgagtgcagcctggtggtaaaagaaccagagctttctaacaagactctgcctaaagctccatactcatttaccttgaactcaccattcacatttcaaaagaaacataaatggattgagattgatgtgacggccatccttcagcccctagtggtctccaacaagagagagattcacatgtccatcgactttacgtgtgtgaaagaccagctccagcatctttcagcacaggaaagtccatgtaacatgactcttctggcagccccctcactgcttttatatctgaacgacacaagcacacaggcctatcacaggtggtattccctcccctggaaaaggaggccttcccagggtcctgaccagaaaagagggctgtctgagggtgtaccatcctctcgtcaccggagagctcaggacactgtaagctctgaattgaagaagcctctggttccagcttcattcaatctgagtgaatacttcaaacagtttctttttccccaaaacgagtgtgaactccacgactttagactgagcttcagtcagctgaagtgggacaactggattgtggccccacacaaatacaaccctcgctactgtaaaggggactgtccccgggcggtcgggcaccggtatggctctccggttcacaccatggtgcagaacatcatccacgagaagctcgactcgtcagtgccgcgaccatcctgcgtacctgccaagtacagccctttgagtgtgttggccattgagcccgatggctccattgcttataaagaatacgaagacatgatcgcaacaaagtgcacgtgtcgttaa

SEQ ID NO: 53 - DNA, 1371 bp, Sus scrofa
  source 1..1371: mol_type = other DNA
cctgctagttcttccaaactatggcgcttcccagaaaattcttcctttgtttttgctgctttgccttgttctgttttcctgttagctgtggttctcaggcttctagaggagaagctcactttgcaactagtgctgcgttggaatctgaggctgagccttggtccttgctgcggcctccagatgagagacacagatctggcctcccttcccccctcttcaatgttctgtatgatgggcatggggggttgcccaggctgcagccagactccagagctttgcgctacatgaagaggctgtataaaacatatgctaccaaggaggggatccccaaagccaacagaagtcacctctacaacactgtccggctcttcacaccctgtgcccagcataagcaggctcctggggaccaggtgacaggaacccttccatcaggggacctgcggtttaacctggatcgtgttactgctgttgaacatttactcaagtcagtcttgttatacacgtggaacaactccatttcttttccctcccctgttaaatgtgagtgcagcctggtggtaaaagaaccagagctttctaacaagactctgcctaaagctccatactcatttaccttgaactcaccattcacatttcaaaagaaacataaatggattgagattgatgtgacggccatccttcagcccctagtggtctccaacaagagagagattcacatgtccatcgactttacgtgtgtgaaagaccagctccagcatctttcagcacaggaaagtccatgtaacatgactcttctggcagccccctcactgcttttatatctgaacgacacaagcacacaggcctatcacaggtggtattccctcccctggaaaaggaggccttcccagggtcctgaccagaaaagagggctgtctgagggtgtaccatcctctcgtcaccggagagctcaggacactgtaagctctgaattgaagaagcctctggttccagcttcattcaatctgagtgaatacttcaaacagtttctttttccccaaaacgagtgtgaactccacgactttagactgagcttcagtcagctgaagtgggacaactggattgtggccccacacaaatacaaccctcgctactgtaaaggggactgtccccgggcggtcgggcaccggtatggctctccggttcacaccatggtgcagaacatcatccacgagaagctcgactcgtcagtgccgcgaccatcctgcgtacctgccaagtacagccctttgagtgtgttggccattgagcccgatggctccattgcttataaagaatacgaagacatgatcgcaacaaagtgcacgtgtcgttaacacaggctcctctgaa

SEQ ID NO: 54 - DNA, 1795 bp, Bos taurus
  source 1..1795: mol_type = other DNA
agaggggctggaagaaaaacagagtccgtctgcgccagtctcattatattcaaatattcattttaggagccattccgtagtgccatccagagcaacgcactgccgcagctcctctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctccttcgggacttcgaggccacacttctgcagatgttcgggctgcgccgccgcccgcagcctagcaagagcgcagtcatcccggattacatgcgggatctttacagacttcagtctggggaggaggaagaggaagagcagatccagggcatcggtctggagtatcctgagcgccccgccagtcgggccaacaccgtgaggagcttccaccacgaagaacatctggagaacatcccagggaccagcgaaaactctgcttttcgtttcctctttaacctcagcagcatcccagagaacgaggtgatctcgtctgccgagcttcgactcttccgggagcaggtggaccagggccctgactgggatcagggctttcatcgtataaacatttatgaggttatgaagcccccggcagaagtggtgcctgggcacctcatcacacgactactggacacaagactggtccaccacaatgtgacgcggtgggaaacttttgatgtgagccctgcagtccttcgctggacccgggagaagcagcccaactatgggctggccattgaggtgacccacctccatcagacacggacccaccagggccagcatgtcaggattagccgatcgttacctcaagggagtggggattgggcccagctccggcccctcctggtcacctttggccatgatggccggggacatgccttgacccgacgcaggagggccaagcgtagccccaagcatcacccacagagggcccggaagaagaataagaactgccggcgccactcgctctacgtggacttcagtgatgtgggctggaatgactggattgtggccccaccaggctaccaggcattctactgccacggggactgcccctttccactggccgaccacctcaactcaaccaaccacgccattgtgcagaccctggtcaactctgtcaattccagtatccccaaagcctgttgtgttcccaccgaactcagcgccatctccatgctgtacctggatgagtatgacaaggtggttctgaaaaattatcaggagatggtagtggagggatgtgggtgccgctgagatcaggccttctttggggacacacacacacacacacacacacacacacacacacacacatcccatccactactcacccacacactacacggactgcttccttatagctggacttttatcttaaaaaaaaaaaaaaggaaaaaaaaatctaaacattcaccttgaccttatttatgactttacgtgcaaatgttttgaccatattgatcatatattttgacaaaatatatttataactacatattaaaagaaaaaaataaaatgagtcattattttaaaggtaaaaaaaaaaaaaaaaaaaaaaaaaaaaaaaaaaaaaaaaaaaaaaaaaaaaaaaaaaaaaaaaaaaataaaaaaaaaaaaaaaa

SEQ ID NO: 55 - DNA, 1667 bp, Sus scrofa
  source 1..1667: mol_type = other DNA
attatattcaaatattcattttaggagccattctgtagtgccatccagagcaacgcactgccgcagctcctctgagcctttccagcaagtttgttcaggattggctgtcaagaatcatggactgttattatatgccttgtttcctgtcaaaacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctccttcgggacttcgaggctacacttctgcagatgttcgggctgcgtcgccgcccgcagcctagcaagagcgccgtcatcccagattacatgcgggatctttaccggcttcagtctggagaggaggaggaggaagagcagacccacagcgttggtctcgagtatcccgagcgccctgccagtcgggccaacaccgtgagaagctttcaccacgaagaacatctggagaacatcccagggaccagcgaaaactctgcttttcgtttcctctttaacctcagcagcatcccagagaacgaggtgatctcatccgcagagcttcgactcttccgggagcaggtggaccagggccctgactgggagcagggcttccatcgtataaacatttatgaggttatgaagcccccaccagaagtggtgcctggacacctcatcacacgactactggacacgagactggtccaccacaatgtgacacggtgggaaacttttgatgtgagccctgcggtccttcgctggacccgggagaagcagccaaactatgggctggccattgaggtgacccacctccatcagacacggacccaccagggccagcatgtcaggattagccgatcgttacctcaagggagtggggactgggcccagctccggcccctcctggtcacatttggccatgatggccggggacatgccttgacccgacgccggagggccaagcgtagccccaagcatcacccacagagggctcggaagaagaataagaactgccggcgccactcgctctatgtggacttcagtgacgtgggctggaatgactggattgtggctccaccaggctaccaggccttctactgccatggggactgcccctttccactggctgaccacctcaactcaaccaatcatgccatcgttcagaccctggtcaactctgttaattccagtatccccaaagcctgttgtgttcccaccgaactgagtgccatctccatgctatacctggatgagtatgacaaggtggtactgaaaaattatcaggagatggtagtagagggatgtggttgccgctgaatcaggccttccgtggggagagacagacagacacacacacacacacgccccatccactactcactcacacactacacagaccgcttccttatagctggacttttatctttaagaaaaaaaaaaaaaaaaggaaaaaaccctaaacattcaccttgaccttatttatgactttatgtgcaaatgttttggccatattgatcatatattttgacaaaatatatttataactacatattaaaaaataaaatgagtcattattttaaagataaaaaaaaaaaaaa

SEQ ID NO: 56 - DNA, 7147 bp, Sus scrofa
  source 1..7147: mol_type = other DNA
cggtgcatgctttctagtacctctgcacgtggtctccaggtgagccccggcagcttacccgtgcgggaggcagcggcgtcccagcccgggcggcagctggggactcgggcgctgcccgggtttccgggaccagggcctgcgaggcgaggccccggcggctggatgggaggatgtgggcggggctcccatcccagaaagagagaccagcgagggaggagggaagaagggaggggctgctggagaagaagaggaggaggaaggaaagaaagaaagggagagggaaagaggaggaagaagatgcaagagggcagaggaggagggagggaggtaaagggagtggagctctgcccggaagctaggtgagttaggcatcccagccgagggaagggagcctgccacactgctgctgatcccggctgtctcgcctttctccctgatgggattcccgtcggcgccttctcgagcctgctgcgccccagttcccggccctcgcccaggttcactgcagccgttcagaggtctccaggaactgcaactggggagcccgctaccgcagggacctatggtgagcaaagctacctggcaaggggctgaggcgcatccttgggaggggggagagggtggctctggctgtggcgaaaggtggccccctttttcccttcctagcggtgttgctagaggtagggatgctggcgctgagagacatttccgtatatgctacagcctctacccggaccacttttcccgtccggtggcccggggcgattaccacctttggtaggttcatggcaggagagaaggggagaatagaagcctttagttggagatcgttgggaaagactgtggggtcttgagcggaacacgtcgcccttggactagctttggcaatcccaggacggaaagggccgggagcgaagggggagtgacggctctgagatccagagagagtgagggggctgatgggcggcgagagtacacctaactaaggctggttctgcagtggggagcgcagttctgcttctggggcgtgcgtgcgctgcaggaggggctgcgtctaggggagtgtcacccgacccggcactcagccggcgaaaacccctgagagctggcgaactgccgctgcccgacgcttcttctccatgagctatttattatcttggacttggcggcatccaaggagcctctcctctatcatttcaccgggtagtggctgtagggaggccaggatgctagctgctctgggtacgcggcttcttgaggtgtcgtgtcctttggggaggcactggggagatgcgtcgtggactcgtggctcttactgaccctcccggccccaaaaccaactcgaaagggtaccgggattcacgaatctttgcggacccatccccatggcctcagacgcttcccgggcaccgatttcgtggtaggtttctgaggatcgcgttttgtgagctcaaggctttttgagatgttaaaaccccgaaaaacagattcggtaaactgaggtcacctctgaacaaattttggcgttgggagaaaactcgtagggaagtgaaggacagtcgccatgagttgcatctccacccctcaaaaaaaaaaaaaaaaaaaaaagaaagaaagagagagaaagaaaaaagctggagacgaaagtgcttttcagctctttttttaattaaaaaaaaatgtgattagagacgaatcctattgagtccgaaattgaaacaacccgctcttccctcgcctctgagttttagcgcagcgcgctcccgccgtagccctgggatttctagggtcggaggattgtccaggaactgtgggcgcccgccccttccactgtccttcgcccgcggggagctgcccttctctgcttctcccatctctggagctttcccccccaccccggtgcattttaagggttccaggcgtctcaacgaaactctttgctcttcccacccgttcgcagtcagcctcagcggtggcgcggtagcgggaaaacttaaaggcgctcaaaaggcggcagctctgggcgggcaaggatcgtcgcgcaggggcgcccgcgcacacccaggccagcgacgggtagggccgcgcgggacaaccgcggccgggagggagggctaagcgtttgatccttcacacttcttccaggccctcctgcaagtgcccccaagataaaaccgagttttgctgggtgaggccgggaacctgagccttcgagctctgaatagacccaaggcagaaactgggaacaggcttaggaaagggcggcagggtacagtgtgctgtgcagccgggatcgcgcgactgtgacccctgcgcaggccgggcagattcagtttcgagggtcacggtttcggaaaggaccctgtattgagtgatccacgcatgcagcatctttggatagagtcgggatatcacagtaataccttgagcggtgggttggtaaaattcttgaagcatttgctgagattctgaagctggtgcaaaggaggaagggggggggaggaagtggcgggagaggagtagtggtttaaaaaagacgagctggggtgggggtggggggaaagacagacgcagagatcgagcgtaggccgaaagctgttcacggttttctggactcctgggaacccattgtgatttcctttctgccctggctcgggagttgtaaaacctctgagacttggagacctgaaacaagtgttgagcccttccgaggagacgcctctcttttgagtccgcccctccgcctttaagggcctcaggaccgctttctttggggatttactttgctcggtgggcacgccgtggtgtggagattctctccgtaaaggaaccggaggaaagagcccagcggccattcccttaccacgttcccacactggtcaccgcctgaccatctacccctccgaccacttatgacaggtcctcagcttttggtcgggagctaaagaggctgctcaattcctctaaacactcgcagaagttcgccagggggtgactgtagctgggagcaatttttggcctctcggagatcctggggagaaagaggggtgggtgcgcgcggcacgtgtatatctgtctttcagtgcttttctttttaaacgaccctgggtggtgcggctttcagctgctgagactccgccgcccccaaatcccggcccaacttgagccaggctcacactgaaaggcgaagcccctaccgtgctccccagatcctctcgcctaaggcagctggcgggagggatcaggccgacacgacggaggctgcaggtagaggcgaggaccacgtcctccgccagctgcacccggggaccagctgaggactcggccgggagctcctcccggacgctggtggttggggcagcaggcggggaggctggcctctcagagagggggcggggcggagatggggcgggtccactgcggggaaggcggacgcttcggtgcgcggggcggagtctttgcattcacgtgactcagaggggctggaagaaaaacagagtcagcctgcgccagtctcattatattcaaatattcattttaggagccattctgtagtgccatccagagcaacgcactgccgcagctcctctgagcctttccagcaagtttgttcaggattggctgtcaagaatcatggactgttattatatgccttgtttcctgtcagtgagtagacacctcttccttccctcttcccgggaattcactctgccctccccacccgtgctcgccttgtgtccctcccgtcggacctgccttccagagtccacactccgctttctggcagcgctgtcgctttcttcttggcgccgcagccgctgcgctctgagcgtaccggttctatttaaagtgctgcttgctccactctcttcctgatctgtgcgttgcaaggaaggagagcgaaagggggcaaaaaagaaaagtccgcagccaggggtgcttaccatccccttctcccacttgactgcggatcacccagtgcccccgtggcatattcccagctgcttgtcaaactcagacgtcgctcttggaatcatcccccccacacacccttctctgggagtgagtgagcgggctcaatcaaacgctttttgctgggcatttcaaattcatcagcccccgtaaaataaaccttccagacccagccgccgggtgtcttcgccctgcccctgcttctctgttttcccttcctcctgctccgaaccagctggaagttgtggaagtcggctcggagggcggaggaagaagggggggtggaggaaggggggggggagagactgaaggtttgaagtggagagtagagagatggtattttaattctccctcccccatccttttttacctcctcaatgttaactgtttatccttgaaaaagccaagctgagatcatggctcaggtagcagttgggacaaaaaaagattaacaggatggaggctatctgatttggggttatttgactgtaaacaagttagaccaagtaattacagggcaattcctactttcaggccgcgcatggctgcagctggtggtggggtgtgtgtgagggggaagacacaaacttgatctttctgacctgttttacatcttgaccctctctgtctagccctgtatgagtttgctgagacatctctatttttctctagttattggtgtttatttattctttaaccttccacctcctccccctccctagagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctccttcgggacttcgaggctacacttctgcagatgttcgggctgcgtcgccgcccgcagcctagcaagagcgccgtcatcccagattacatgcgggatctttaccggcttcagtctggagaggaggaggaggaagagcagacccacagcgttggtctcgagtatcccgagcgccctgccagtcgggccaacaccgtgagaagctttcaccacgaaggtcagtcccagctcagggtctgggctgtggagggccggtagggcagctctgggggagaggagttcaggttacatcaaagccccagatcaggaggctagggaaccgagctgctcacctccaaggattttcagagctgtggcccagtttattttttggagacggaggggaggggtttgggaggagaatgacaccgctcaagtctctggctagccccagaggtgtgcttctgtgatatcaaagccttttatgccagattttctgcgcatttttttttccaaagcacctactgaatttaatattacagctgtgtgtttgtcaggtttattcaataggggccttgtaatctgatctgaatgtttcctagcggatgtttcttttccaaagtaaatctgagttattaatccaccagcatcattactgtgttggagtttatttccctctctgtaacatgatcaacaaggcatgctctgtgtttccaaagatcactggggaaatgtttggtaacatactcgaaagtggaagaagagggagagggtggttgtgtgcatgttccctcctgcctctactctgttggcccctctttttctttacaaccacttgtaaagaaaactgtgtacacaaagccaggagggctttaaaaggggagtcttatggtggtggagtaaggagttgacacatggaaattattagacatataaaggaggttgggagattgtctttggtgcctgctgaatgctatctaagcttcactagtttgctagctgccccattgatctgctctttcaaattaaaaggtatgtttatttccccctagtaggcttccactgtataagcagagttccccactcatcacccagctcttagctgtttcttgacttttcagtctctcaaaaagctcatgggcttttttttcttttctcttttctttttcctccccaactgtgcctagaacatctggagaacatcccagggaccagcgaaaactctgcttttcgtttcctctttaacctcagcagcatcccagagaacgaggtgatctcatccgcagagcttcgactcttccgggagcaggtggaccagggccctgactgggagcagggcttccatcgtataaacatttatgaggttatgaagcccccaccagaagtggtgcctggacacctcatcacacgactactggacacgagactggtccaccacaatgtgacacggtgggaaacttttgatgtgagccctgcggtccttcgctggacccgggagaagcagccaaactatgggctggccattgaggtgacccacctccatcagacacggacccaccagggccagcatgtcaggattagccgatcgttacctcaagggagtggggactgggcccagctccggcccctcctggtcacatttggccatgatggccggggacatgccttgacccgacgccggagggccaagcgtagccccaagcatcacccacagagggctcggaagaagaataagaactgccggcgccactcgctctatgtggacttcagtgacgtgggctggaatgactggattgtggctccaccaggctaccaggccttctactgccatggggactgcccctttccactggctgaccacctcaactcaaccaatcatgccatcgttcagaccctggtcaactctgttaattccagtatccccaaagcctgttgtgttcccaccgaactgagtgccatctccatgctatacctggatgagtatgacaaggtggtactgaaaaattatcaggagatggtagtagagggatgtggttgccgctgaatcaggccttccgtggggagagacagacagacacacacacacacacgccccatccactactcactcacacactacacagaccgcttccttatagctggacttttatctttaagaaaaaaaaaaaaaaaaggaaaaaaccctaaacattcaccttgaccttatttatgactttatgtgcaaatgttttggccatattgatcatatattttgacaaaatatatttataactacatattaaaaaataaaatgagtcattattttaaagat

SEQ ID NO: 57 - DNA, 1671 bp, Sus scrofa
  source 1..1671: mol_type = other DNA
gaaaattatattcaaatattcattttaggagccattctgtagtgccatccagagcaacgcactgccgcagctcctctgagcctttccagcaagtttgttcaggattggctgtcaagaatcatggactgttattatatgccttgtttcctgtcaaaacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctccttcgggacttcgaggctacacttctgcagatgttcgggctgcgtcgccgcccgcagcctagcaagagcgccgtcatcccagattacatgcgggatctttaccggcttcagtctggagaggaggaggaggaagagcagacccacagcgttggtctcgagtatcccgagcgccctgccagtcgggccaacaccgtgagaagctttcaccacgaagaacatctggagaacatcccagggaccagcgaaaactctgcttttcgtttcctctttaacctcagcagcatcccagagaacgaggtgatctcatccgcagagcttcgactcttccgggagcaggtggaccagggccctgactgggagcagggcttccatcgtataaacatttatgaggttatgaagcccccaccagaagtggtgcctggacacctcatcacacgactactggacacgagactggtccaccacaatgtgacacggtgggaaacttttgatgtgagccctgcggtccttcgctggacccgggagaagcagccaaactatgggctggccattgaggtgacccacctccatcagacacggacccaccagggccagcatgtcaggattagccgatcgttacctcaagggagtggggactgggcccagctccggcccctcctggtcacatttggccatgatggccggggacatgccttgacccgacgccggagggccaagcgtagccccaagcatcacccacagagggctcggaagaagaataagaactgccggcgccactcgctctatgtggacttcagtgacgtgggctggaatgactggattgtggctccaccaggctaccaggccttctactgccatggggactgcccctttccactggctgaccacctcaactcaaccaatcatgccatcgttcagaccctggtcaactctgttaattccagtatccccaaagcctgttgtgttcccaccgaactgagtgccatctccatgctatacctggatgagtatgacaaggtggtactgaaaaattatcaggagatggtagtagagggatgtggttgccgctgaatcaggccttccgtggggagagacagacagacacacacacacacacgccccatccactactcactcacacactacacagaccgcttccttatagctggacttttatctttaagaaaaaaaaaaaaaaaaggaaaaaaccctaaacattcaccttgaccttatttatgactttatgtgcaaatgttttggccatattgatcatatattttgacaaaatatatttataactacatattaaaaaataaaatgagtcattattttaaagataaaaaaaaaaaaaa

SEQ ID NO: 58 - DNA, 1320 bp, Ovis aries
  source 1..1320: mol_type = other DNA
gtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctccttcgggacttcgaggccacacttctgcagatgttcgggctgcgccgccgcccgcagcctagcaagagcgcagtcatcccggattacatgcgggatctttacagacttcagtctggggaggaggaagaggaagagcagatccagggcatcggtctggagtatcctgagcgccccgccagtcgggccaacaccgtgaggagcttccaccacgaagaacatctggagaacatcccagggaccagcgaaaactctgcttttcgtttcctctttaacctcagcagcatcccagagaatgaggtgatctcgtctgccgagcttcgactcttccgggagcaggtggaccagggccctgactgggagcagggctttcatcgtataaacatttatgaggttatgaagcccccggcagaagtggtgcctgggcacctcatcacacgactactggacacaagactggtccaccacaatgtgacacggtgggaaacttttgatgtgagccctgcagtccttcgctggacccgggagaagcagcccaactatgggctggccattgaggtgacccacctccatcagacacggacccaccagggccagcatgtcaggattagccgatcgttacctcaagggagtggggattgggcccagctccggcccctcctggtcacctttggccatgatggccggggacatgccttgacccgacgccggagggccaagcgtagccccaagcatcacccacagagggcccggaagaagaataagaactgtcggcgccactcgctctacgtggacttcagtgatgtgggctggaatgactggattgtggccccaccaggctaccaggcgttctactgccacggggactgcccctttccactggccgaccacctcaactcaaccaaccacgccattgtgcagaccctggtcaactctgtcaattccagtatccccaaagcctgttgtgttcccaccgaactcagcgccatctccatgctgtacctggatgagtatgacaaggtggctctgaaaaattatcaggagatggtagtggagggatgtgggtgccgctgagatcaggccttctttggggaca

SEQ ID NO: 59 - DNA, 1230 bp, Ovis aries
  source 1..1230: mol_type = other DNA
atgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctccttcgggacttcgaggccacacttctgcagatgttcgggctgcgccgccgcccgcagcctagcaagagcgcagtcatcccggattacatgcgggatctttacagacttcagtctggggaggaggaagaggaagagcagatccagggcatcggtctggagtatcctgagcgccccgccagtcgggccaacaccgtgaggagcttccaccacgaagaacatctggagaacatcccagggaccagcgaaaactctgcttttcgtttcctctttaacctcagcagcatcccagagaatgaggtgatctcgtctgccgagcttcgactcttccgggagcaggtggaccagggccctgactgggagcagggctttcatcgtataaacatttatgaggttatgaagcccccggcagaagtggtgcctgggcacctcatcacacgactactggacacaagactggtccaccacaatgtgacacggtgggaaacttttgatgtgagccctgcagtccttcgctggacccgggagaagcagcccaactatgggctggccattgaggtgacccacctccatcagacacggacccaccagggccagcatgtcaggattagccgatcgttacctcaagggagtggggattgggcccagctccggcccctcctggtcacctttggccatgatggccggggacatgccttgacccgacgccggagggccaagcgtagccccaagcatcacccacagagggcccggaagaagaataagaactgtcggcgccactcgctctacgtggacttcagtgatgtgggctggaatgactggattgtggccccaccaggctaccaggcgttctactgccacggggactgcccctttccactggccgaccacctcaactcaaccaaccacgccattgtgcagaccctggtcaactctgtcaattccagtatccccaaagcctgttgtgttcccaccgaactcagcgccatctccatgctgtacctggatgagtatgacaaggtggttctgaaaaattatcaggagatggtagtggagggatgtgggtgccgctga

SEQ ID NO: 60 - DNA, 1644 bp, Bos taurus
  source 1..1644: mol_type = other DNA
acattggactggatggctcttatcttacaggtgactcctttcattccgtcttttttaaataaatactttcacacccggagatcttcacctttctctaagtttcttctacctagattcttcagcctggacttttcctagcatctacctcttaggttttttccttctccttcaactcagcaacatgagtgtgggcccagcttgtccccaaagcctgcttggccccgaagcatccaactctagggaatcttcacccatgcctgaagaaagttacgtgtccttgcaaacgtcatctgctgacaccctcgacacggacactgtctctcctcttccctcctccatggatctgcttattcaggacagtcctgattcttccacaagccccagagtgaaaccactgtccccgtctgtggaggagagcacagagaaggaagagacggtcccggtcaagaaacaaaagattagaactgtgttctcgcagacccagctgtgtgtgctcaatgacagatttcagaggcagaaatacctcagtctccagcaaatgcaagaactttccaacatcttgaacctcagctacaagcaggtgaagacctggttccagaaccagagaatgaaatgtaagaaatggcagaaaaacaactggccgaggaatagcaatggcatgcctcaggggccagcaatggcagaatacccaggcttctattcctaccaccaggggtgtttggtgaactctcctggaaacctgcccatgtggggtaaccagacctggaataaccccacgtggagcaaccagagctggaacagtcagtcttggagcaaccactcctggaacagtcaggcctggtgcccccaagcctggaataaccagccttggaacaatcagttcaacaactacatggaggaattcctgcagcccgggatccagctccagcagaattctcccgtctgtgatctggaggccaccctgggaactgctggggaaaattataacgttatacagcaaactgtcaagtatttcaattcccagcagcaaatcactgatttattcccaaactaccctctcaacatacagcctgaagatttgtaacgtctatttgtgacggctattgtatgggatcttaatgtgagcagagactgtattctctagatttttttctcattctagaattctgtaccacttccccttagtaagctgcttttcactataccttctgtgtcaatttgagggaagggtaatgagtccagtaagaggtttcagaagcattgtataacatgacaaaaggtgtctggctatcgataactcgattataatactttggatataatatataatattttggatacttttaggatctagaatctaactttaagcacagggggcaaaagtacaaagacgtatgatgaggatttttcatattttctggcatagggaggctttacttgctaagagtcccagtccttaagatgaagagtgctttactgatttcctccaccccttttagccattttactacaacgcctaatttgttcatcactttttggagaacgatattttgtattttgccacatcataagtagtaccagtttggctggtttgttgaagtacaaataaaaaatagccatttcaaaaaaaaaaaaaaaaaaaaa

SEQ ID NO: 61 - DNA, 1644 bp, Bos taurus; identical to SEQ ID NO: 60
  source 1..1644: mol_type = other DNA

SEQ ID NO: 62 - DNA, 10971 bp, Sus scrofa
  source 1..10971: mol_type = other DNA
gaattcttgaatctggaatttgggaatggagaaaattgaaaactgtagatgttatcttatccataaatatgttagtgctcaagtccaatattaaaagttatgcctttggaagagaaattaaaaaaaaaaaaaaaagtgaggatggagtggagtttgacaggagtgcctggacagagaaggaaaaaccaattaaataaagagccagagaggagttcctgtcgtgcctcagtggttaaccaaccctactaggaaccatgaggtttcaggttcgatccctggccttgctcagtgggttaaggatcttgcattgctgtgagctgtggtgtaggctgcagacgtagcttggatcccgagttgctgtagctctggtgtaggccagtggctatatctacgattagacccctagcctgggaacctccatatgccctgggagtggccctagaaaagacaaaaaaaaaaaaaaaaaaaaaaagaaaaagccagaggggggaaaagcaaaatgcaagaggaagatgaccagcttgggaaggcgggacagagggacagacagaagggccaacatcggttctaaattttgaaactgtcatatttgctttggcaggtccattacaggaatatcaattgccaagagaatgttcttttttccccctcttttctttctttctttttttttttttgagttttttttttttttttgcagaacacattctagtttttattaaaaacctccagcaaggcaagttacctttagtgcaatctgtgatgttatccatattaaaatctaaaatgtttttgcatacaaaatggagttatcagtgacattagaagaatgccttaaagacaaatttcctggaaacaaagttgaagggtgttttgttcttgatgaacatagaaaaagcagggtgacaatacaagctggctcaggacatcagtccattgccatatattttgctcttgaaaaataacagtttaaagaattttattttgtttgtttggttttttttttgtttttttttttatctttttgctatttctttgggccgcttccgcggcatatggaggttcccaggctaggggtccaatcggagctgtagccactggcctacgccagagccacagcaacgcaggatccgagccgagtctgcaacctacacacaccacagctcacggcaacgctggatcattaacccactgagcaagggcagggaccaaacccgcaacctcatggttcctagtcggattcgttaaccactgcgccaggacgggaactccttaaagaattttattttacacgcaaatgaattttgaatgtactcaattcattaaacaaggtcatttgtcagtgtgagaataatatagtggcacaaaagggaaatttaaattagcattttataattgtatcattgataaattaatgctatatcttgttttaaagaatttttttatattactcaatgattttattacctttatagctgtacaatgatcatcacaacccaggtttatagcagagatttttttaaatataatgatttatattttttccattatagctggtttacagtgttccgttgattttctcctgtacagcaacgtgacccagttacccatacatgtatacattcttttttctcacattatcatgctccatcaccatcacaagtgactagactgagttcccagtgtctccctcttttcaattgggttggtttataggtcaataaattttcttattttatattggttaatctaataacgtcccaggtcaaagaaggagatggtgctctcattctatcacgaactgttaggtttcatgtttgatggattaaaataaaaagcaaaccaggagttcccagcagggctcagcagaaacaaatctgactagcatccatgaggacacacgttccatccctggccttgctcaggtgggttaaagatccggctattgcaatgagctgtgatggaggtccaagcctctgttcagatctgacatggttgtggctgtggtgtaagagccagtggcaacagctctgactggacccctagcctggggacctgtgggtgcggccctaaaaagaccaaaaaaaaaaagcacaaccaaagggtaatttcaaaacttagaggtaggtcacaggcataggatcattgaaagaatttctattaacagctaccatttgtagctcgctgatgtgtcaggtgttatgagtttctagcactttattcgccctgattcttacaacacagagaaggaaggtactgtcatctctatttatttatttttgtctttttgccatttcttgggctgctccctctatggaggttccccgtctaggggtctaatcagagctgtagccactagcctatgccagaaccacaacaacacgggacccaagccgagtctgcaacctacaccacagctcatggcaacgctagatccttaacccactgagcaaggccagggatcaaacctgcaacctcatggtttctagtcggattagttaaccgctgagccacgatggaaactcctgtcatctctatttataactaaggaaactgagacattaaaatggctgagccgagctgttatccagtactgtgtgtacaaaagcgacttaaaaatcctccagagtaataatggtttatatgtaatgactaaccggacacctagggtcctatgtctgcaagtcctttcctctggcttttgtcctgctgaaaagacttgcgtgtctttctgaaagctaaaaacacgagaaagaagtaaagtgggagttaaaaacaagtattatttttaccatcacttgatgcgccaacattcaacatgtgttttattgttaccttagggtgaatcaaagagaaaaatggagctaacatgtttctgcagaataagcctgaactggagacccaaaggagtctcaggtcaagaaattcattgtcccagtgggagtttcagtcaccggaaatagcctcaggaactgaaggtgcatcttccatttgatctgattttttttttttaatttttaaaaaatttttttgcatctttgattttaaaaagtggaaacaaggtggacctgcaagtagttcactgcggggtttattttgtttccaggttccatggtccccgttccccacccagtctgggttactcagcagccctctctttgcattacaatggccttggtgaggctggcagacaggattaactgggaattcgcaagggtgtgtgtgggcgtggggctgccaggaggggcgggcttaagtatggtcgatccttccttataaatctagagcctccaaaatttttctcattttgctagattggggtggttagctcctgttctcgtaagggtgactcacttcatcccattttttgatacttttaacacctggaggagatcttcatgattctaagctcttctatctagacacttaagcctggacttttcctacaatccagctctttggtggtttttttttttttcccccttcttcaactcaacatgagtgtggatccagcttgtccccaaagcctgctttgccccgaagcatccatttccagcgaatcttcaccaatgcctgaggtttatgggcctgaagaaaattatgcctccttgcagatgtcatctgctgagaccctcgacaccgagactggtaagaaaatcttgaatcttgacaatcttgaaaaattgaaaagctaagctgcttcagggaaatagaaagaaggaaaggggccgagggtcgcctgtttcaccttgagcccatcagtataggcaggtgtccttatctattaatcctctcgtcacctctttttcaactgtaacttgataaggtcatgccctttcccctgtgtcccaggcatcgggaactgtggacagagagacgagggattctgcaggccacctgaatttttttttttttccttcccttttagggccgcacccacggcatatagatgttcccaggctaggggtcaaattggagttcagctgccggcctacaccacagccacagcaatgcgggatccaagccgcatcttggacctacaccaccgctcacagcaacactggatccttgaccgactgagtgaggccagggatctaacccgcatcctcacggatgctagtcagattcgtttccttggcaccacaatgagaactcctgtatggagtatttgaaggctgactgcacaggcactgaggaaaggaaatgtgttgtgatcgactgacgtgtaaatcttacatggtatcactaagtccaagtcctgacatcacgcttgaggcggtgagggtcaccttttgggtttgtctatttcaggcatgccggaaatggcttcgctaatttaatcgtgtgaagcgcaccttgtagaaatggtttgtctttctattacagtggcaactggcactcccaacctatgagggtattaactgggcagcttgcgtttgtgagcacctgttatggtcaagtcgcctaaaccccaaggaagtgttcaccctgttgcatggtagatgattccagaagatggaatatgctgacagccaaagtggctcttcagaagagctcagatttgaaattaagtttggcttcaggagttcccgtcgtggcgcagtggttaacgaatccgactaggaaccatgagtttgcgggttcggtccctgcccttgctcagtgggttaacgatctggcgttgccgtgagctgtggtgtaggttgcagacgcggctcggatcccgcgttgctgtggctctggcgtaggccagtggctacagctctgattggacccctagcctgggaacctccatatgccgcggaagaggcccaagaaatagcaaaaagccaaaaaaaaaaaaaaaaaaaagtttggcttcaaaaatttatactcaggtgctcaggcttttaactggtaactgcgggtgtgcagggtgctcggcatnctataaaggcaggactacctcttanttttttcttttacatttttggtcaccctgtgacctatggagtttctagccaggggtcaggctggagctgcagctgcagcaacgccggatcctttaacccactgttctggcctgggaacaaacctgggccctgaccctgcagagacaccaccagttccatgtgccatagtgggaactccactatcccagcaccacctcttttggaaatcaatttggcagaagttgttagaaataaaaaggtaggcgttctcaagtggctcagtgggttaaagatgcatcgttgtcactgctgcggctcgggtttgaaccctggtccaggcacttcatatgctgtaggcttagccccccccaaaaaggtagtaattacaaataaaaaacatatgggagttccctcacgtagctcagcggttaacgaacctgactaggatccatgagggaggggggtttcgatccctggcctcgctcagtgggttaagggatccggcattgccttaagctatggggtaggtcacagatgctgtttggatcctctgttgctgtggctgtggcgtaggctggcagccataaaaggcaaaaataaacaaaccaaggtatggagttccccgttgttctggctcagaaggctaagaatctgatatagtgtccctttggcctcactcagtgtggcttgagggtctagcattgtcgcaagctgtggctgcggtgtaggccagcggctacagctcccattaaaccgctagcctgggaacctccatatgccatgggtgtggcctcaaaaagacaaaaaataaataaaaataagacgtgagtcactatattgtacacctgtaatttatagagtattatacaattatacttcaatagaaaacagtaacttccttggtggtctagtgtttgggattcgattctttgacaaggttatggcccaggttccataccttgtctgggaactgggatcccacatcaggccgaggtgtgctgcgggcaaaaaataaatagtaaggacttcccgtcgtggctcagtggttaacgaatccgactaggaaccaccaggtttcgggttcgatccctggccttgctcagtgggttaaggatccggcattgccgtgaactgtggtgtaggttgcagacgaggctcagatcccgtgttgctgtggctctggtgtaggccagtggctatagctccgattggacccctagcctgggaacctccatatgctgcgggagcagcccaagaaatggcctccatatgctgcgggagcagcccaagaaatggcaaaaagacaaaaaaaaaagtaaaagattatgattggcttgtcccaaatgccaaagtaaaacgaacccaaaaactccatgaaggaggaatgactttggaaatgaggtcacaaagttaatgaaggtaaagtctggatggaaacagggctggtcagaattcatattaccctaccaggcctggtatcagaactgggcagtatgctcttgattttataggtccctggaatcgggatcatgttttaaaaattcagcgggtgaagactgatggaaaaaaagttatggggggaacacaggagggggagtcaagtgatcattgtgacctttgagaacagccccgggtgtgggctcctgagcttagatgggcaggaggaagtggttttgagcccctctctgctttccagcgatgcataatatcagccatgtccatggtttgtcaaacagcactgcacaaaggcgagcaaggtgttcagggtcacttttaagattaaaatcagtacttaactacctagctctgaggttgtgagaactctaatattagtgctttgaacacaagacatttttaatattggaagttttagagaaaagtgtcattttattcccaacagtctctcctcttccttcctccatggatctgcttattcaggacagccctgattcttccacaagccccagagtaaaaccactgcccacatctgcagagaagagcacagagaaggaggaaaaggtcccagtcaagaagcagaagatcagaactgtgttctcgcagacccagctctgtgtcctcaacgacagatttcagaggcagaagtacctcagcctccagcagatgcaagaactttccaacatcctgaaccttagctacaaacaggttagggttgttttgttgttgatgtaataggaaataaaaataaggccgatttctatgttctgtgtgtgtgtgcaccccgacagataacttcatgtttcatgtgaaaatggaaacttttttttttttttgctttttagggctgcaactctggcatatgcaggtttccaggctaggggtttaactgacgctacagctgccggcctacaccacagccacaagcaacatcagatcggagttgtgtctgcaacctacaccacagctcacagcaatgccagatccttaacccactgagcgaggccagggattgaacccgcagcctcgtggttccttgtcgggttcatttccactgctccatgatgggaactcctgaaaatgtaatctttttttttttttgtctttgttgttgttgttgttgttgctatttcttgggccgctcccgcggcatatggaggttcccaggctaggggttgaatcggagctgtagccactggcctacgccagagccacagcaatgcgggatccgagccgcgtctgcaacctacaccacagctcacggcaacgccggatcattaacccactgagcaagggcagggaccgaacccgcaacctcatggttcctagtcggattcgttaaccactgcgccacgacgggaactcctgaaaatggaatctttaatgtgaaaatggaatcaaatattgtgtgaatgtgttatagtttacttaaccacttttccttgttgcacaaaccaaagttgtggaaacatctttataatcttccttaatcaaaggactatcttctgaatgaggagagtccctaaccaacacgggttctcattaaaattgcccgttttgtttttctctgtgcatttggttacttagtgaggttaatcatttttcatattattagaccttttttttttttctttttttttggtctttttgttatttcttgggccactcctgcggcatatggaggctcccaggttaggggtcgaatcggagctctagccaccggcctatgccagagccacagcaacgcgggatccgagccgcgtctgcaacctacaccacagctcacggcaacgccggatcgttaacccactgagtaaggccagggatcaaaccctcaacctcatggttcctagtcggattcgttaaccactgcgccacgacgggatctcctagccattttattcaatcacatggttttctttttttttttttttggccttttctagggctgcgatcccgaggcatatggaggttcccaggctaggggtcgaatcggagctggtagataccagctacatcagagccacagcaacatgggatctgagctgcatctgcgacttacaccacagctcacagcaatgccggatccttaacccgttgagcaaggccagggatcaaaccctcaacctcatggttcctagtcggattcgtgacccactgcgccacgacaggaactcccagatggttttcttactggtgccatacttagaaagtccttccctattccatgacctttctttttttcccccagctgcactcaagtcacgtgcaagttcccaggccagggagcaaacctgtgctgctgcagtgacaacgctgattccttatcccactgccccacaagagaactcccatgatcttattttacacatacatattcttctacttctgtgatcctgttttctgactcatagtttataaatcatctaaaaattttagtgcattctgtattgtaatataatttaatattttctaaatagttgtccctgtggtatgtttcataattcccgaattaaagtaacatttttatattcaatgtaaatgtgcttacttggagtggggtttcttactgcaaacaaataacgtgttttgttgggtttttttggctttttagggctgcacctgcagcatatgggctaggggtcgaattggacctgcagctgctggcctatgccacagccacagcaacatgggttccgagcctcgtctgcgacctacatcacagctcatggcaacgccagatccttaacccactgagtaaggccagggatcgaacccgcatcctcatggatactagtctcgttccttaaccactgagccacaaagggaactcctaactggttatttctgagtctgagatagttgtagctgccaggggggaaaaaaaagcctaatatctttgatttttagtacccctgtattagtattttgtttttctgttaccttttccttacaggttaaaacctggttccagaaccagcgaatgaaatgtaagaggtggcagaaaaaccactggccaaggaatagcaacagtgtgattcaggtaacaggaaacttatttttctgctctttcctagcaaggactttgtagttttgtccattgctgaagcatgtaatcccaacaaaagacatttctgagtttccttgtgctttttcagttaatccatccttctctttcaaagggctcagccagtacagaatacccgggcttctattcctatcaccaaggatgcctggtgaacgcttctggaaacgtgcccgtgtggggtaatcagagctggaataacccatcatggagcaaccagacctggaacagccagtcttggagcaaccaaacctggaacagccagacctggtgcccccaagcctggaataaccagacttggaatagccagctcaacaactatgttgaggaattcctgcagccccagctccagtttcagcaaaattctcctatcagtgatttggaggccgtcttggaaactgctggggaaaatcataatgtaatacagcagacttcaaagtactgcagtacccagcagcaaatcatggatttattcccaaattactccatgaacatacagcctgaagatatgtgacgatcattttatttttttaaaaaattttattgggagtatagttgatttacaatgttgtgtcaatttctgctgcaccgtaaagtgacccagtcatacatatacacattttttttttttttttttaggcatatgtaggctctcgggctaggggttgattcggagcagctacagctgctggcctaggccacaatcacagcaatgcaggatctgagctgcatctgtgacctacaccacagcttatggcaacgccggattcttaacctactgagaaaggccagggattgaacctgcgtcttcatgggtgctagtcagattcgtttctgctgagccacaaaggcgactccggtatacattctttttctcatattccatcatccagggacttaatatagttctctgtgctctacagtgggacctcattgtttctccattctaaatgtaatagtttgcatctaccaaccctcaaatccctgtctatccctctgcgcgcgcccccctcccccgccttggcaaccacaggtctgttctctacatctgtgcatctgtttctgttttgtagataggttcatttttgtcatattttagattccacatgtaggtgatatcatatagaatttgtcattctctttctgatttcacttagtataagcatctagttgcatccgtgtcattggaaatggcattatttgggggttttttatggctgggtagtaatccactgtatgatgattctattcaatctgggaagtgatagtattttctagaacttttgtttaggtttgtttctctgacttgataaaacctgctctcatgccttctgtgctaatttggaggtggggctgggcaggtataattgaagtctcatcaaagaggcttgagtagcactggcttcttacatggagaacatgataaaagaagcgtctgaatatagcgactagatagaatactatttgctggatgtctttatgattgagaatctaaccctc

SEQ ID NO: 63 - DNA, 1181 bp, Sus scrofa
  source 1..1181: mol_type = other DNA
attttgctagattggggtggttagctcctgttctcgtaagggtgactcacttcatcccattttttgatacttttaacacctggaggagatcttcatgattctaagctcttctatctagacacttaagcctggacttttcctacaatccagctctttggtggtttttttttttttcccccttcttcaactcaacatgagtgtggatccagcttgtccccaaagcctgctttgccccgaagcatccatttccagcgaatcttcaccaatgcctgaggtttatgggcctgaagaaaattatgcctccttgcagatgtcatctgctgagaccctcgacaccgagactgtctctcctcttccttcctccatggatctgcttattcaggacagccctgattcttccacaagccccagagtaaaaccactgcccacatctgcagagaagagcacagagaaggaggaaaaggtcccagtcaagaagcagaagatcagaactgtgttctcgcagacccagctctgtgtcctcaacgacagatttcagaggcagaagtacctcagcctccagcagatgcaagaactttccaacatcctgaaccttagctacaaacaggttaaaacctggttccagaaccagcgaatgaaatgtaagaggtggcagaaaaaccactggccaaggaatagcaacagtgtgattcagggctcagccagtacagaatacccgggcttctattcctaccaccaaggatgcctggtgaacgcttctggaaacctgcccgtgtggggtaatcagagctggagtaacccaacctggagcaaccagacctggaacagccagtcttggagcaaccaaacctggaacagccagacctggtgcccccaagcctggaataaccagacttggaatagccagctcaacaactatgttgaggaattcctgcagccccagctccagtttcagcaaaattctatcagtgatttggaggccgtcttggaaactgctggggaaaatcataatgtaatacagcagacttcaaagtactgcggtacccagcagcaaatcatggatttattcccaaattactccatgaacatacagcctgaagatatgtgacgatcattttatttttttaaaaaattttattggaatatagttgatttacaaaaaaaaaaaaaaaaaaaaaaaa

SEQ ID NO: 64 - DNA, 1065 bp, Ovis aries
  source 1..1065: mol_type = other DNA
tcttattttacaggtgacccctttcatcctgtctttttttttaatactttcacacctggggatcttcatctttctctaagttcttctatctagattcttcagcctggacttttcctagcatctagctcttaggcttttttccttctccttcaactcaacaacatgagtgtggacccagcttgtccccaaagcctgcttggccccgaagcatccaactctggggaatcttcacccatgcctgaagaaagttacgcgtccttgcaaatgtcatctgctgacaccctcgacacggacactgtctctcctcttccttcctccatggatctgcttattcaggacagtcctgattcttccacaagccccagagtgaaaccactgtccccatctgcagaggagagcacagagaaggaagagaaggtccccgtcaagaaacaaaagatcagaactgtgttctcacagacccagttgtgtgtgctcaatgacagatttcagaggcagaaatacctcagtctccagcaaatgcaagaactttccaacatcttgaacctcagctacaagcaggtgaagacctggttccagaatcagagaatgaaatgtaagaaatggcagaaaaacaactggccgaggaatagcaatggtgtgcctcagggcccagcaacagcagaatacccgggcttctattcctaccatcaggggtgtttggtgaactctcctggaaacctgcccatgtggggtaaccagacctggaataaccccacgtggagcaatcagagctggaacagtcagtcttggagcaaccactcctggaacagtcaggcctggtgcccccaagcctggaataaccagccttggaacaatcagttcaacaactacatggaggaattcctgcagccagggatccagctccagcagaattcccccgtctgtgatctggaggccaccctgggaactgctggggaaaattataacataatacagcaaactgtcaagtatttcagttcccagcagcaaatcactgatttattcccaaactaccctctcaacatacagcctgaagatttgtaa

SEQ ID NO: 65 - DNA, 1020 bp, Ovis aries
  source 1..1020: mol_type = other DNA
tcttattttacaggtgacccctttcatcctgtctttttttttaatactttcacacctggggatcttcatctttctctaagttcttctatctagattcttcagcctggacttttcctagcatctagctcttaggcttttttccttctccttcaactcaacaacatgagtgtggacccagcttgtccccaaagcctgcttggccccgaagcatccaactctggggaatcttcacccatgcctgaagaaagttacgcgtccttgcaaatgtcatctgctgacaccctcgacacggacactgtctctcctcttccttcctccatggatctgcttattcaggacagtcctgattcttccacaagccccagagtgaaaccactgtccccatctgcagaggagagcacagagaaggaagagaaggtccccgtcaagaaacaaaagatcagaactgtgttctcacagacccagttgtgtgtgctcaatgacagatttcagaggcagaaatacctcagtctccagcaaatgcaagaactttccaacatcttgaacctcagctacaagcaggtgaagacctggttccagaatcagagaatgaaatgtaagaaatggcagaaaaacaactggccgaggaatagcaatggtgtgcctcaggggtgtttggtgaactctcctggaaacctgcccatgtggggtaaccagacctggaataaccccacgtggagcaatcagagctggaacagtcagtcttggagcaaccactcctggaacagtcaggcctggtgcccccaagcctggaataaccagccttggaacaatcagttcaacaactacatggaggaattcctgcagccagggatccagctccagcagaattcccccgtctgtgatctggaggccaccctgggaactgctggggaaaattataacataatacagcaaactgtcaagtatttcagttcccagcagcaaatcactgatttattcccaaactaccctctcaacatacagcctgaagatttgtaa

SEQ ID NO: 66 - DNA, 5948 bp, Bos taurus
  source 1..5948: mol_type = other DNA
ggatcctgggatcgcatgggatcgcagagtcggacacaactgaagcaactgagcatgcaccatgcacgcacaagacccaagagagttggaggagccaggtctgatgactcaattggcttgcagtctcctctgggcctgcagagagctggtctgggcagaagtgaagctacctacccttcaggtttaacagaagggtggggatgggaggggagggtgcctcagccttgccagcctcagcaccccaagacatagctggaaggaactgcagacaaacttaactttccactaaggttctgggtcggactccaggcaagcacaggactgatctggctcagatgtctggctgacagccaaccaggaaccctggcaccctcccctcccatctctccctctggtatgggactcttgccaagaaacataccgcttagagcaatcagattactgattgtaatctcaaaagggtattgttgagttgggggaaaggcagaaggtgttttaggggctgctgggaagacagcagtgcaggggccacatgagaagggattctgagagcctagagggtggcgatcgaggcaaggagcattgctgtagatttcctaggtctgcccctccccctcctctgggaggctgtcttcttggaaacagcaaataaatgcatgacaaagggaccctgatggctctgtcttgggggttggggacatggctggggaggggcccctcctggtccaaagcacatctccccacccccaccaggtcccttaatctatcaggtttttttggcaattagtggcttagaggcaaatatagctccagttttgctgccctgtagatctttcatcagacctatgggaagtcttgaaatgcacacatatattagttgcccaaaaagtactcacagatgagctggaactggggctatgtgatacgaatttaggagagagttaagaaaaaaaaaacaggctcaaaagattcctagcatacgaccaaggtgaagacagcaggccacagtcaagaggacccccagcacctcagaaggcagatgctgagcaaatagcctcagacagggcggggatgtagagatgtctaagtcaacagaggtccatgctccaggctcaggattctggccttccaaggtgtatggagctctggttcacagcctttccggagtccccccaattcacctcggggccttcatttggggcaagtttggggagcagacagacaaacatcattcctagcagacaggcaatctgaaagctattcttcttgcaaacagagaataagcactaagccttcagtgtgagccctcaggaccagcccagaccctgtgggagagcttaggtcaggcttcttgcacctccaccccaagtcgctctcctcttcctcctcatctttttgccagcccccctaaacaaggcctggggggcggggggaggggtacaacatgaggctgaatgcctgcgtcctctacagggggacgggatacctaggccccagtgggcggccctgtctgaggctcacagtctttgaggggttggtgggggggtggttgctgcaggagctcttttagctgctctgagggggattcttacggaggggattggggctgggggttggggggcggaagctgtccccaggggagccatccaggcccattcaagggttgagcacttgtttagggttagagctgcccctctggggaccaggattgtccagccaaggccattgtccgggccccttcccccagtccctcccaagcccctttgaacctgaagtcagatattttttttctccccttctacctcctaggcttttcccacccagggcctagggctggaggtctggggtggtaggagggggagggagaaccttcaactgtggttctagatatttgggtctctgaagaggggggcaaggaaacttgatgcacgggacccacagtgagggacccgtggtgtggtgtgtccaatccctctggctggacaaaggtagggagactcaggcctagtcagtccagagcctggccccgaaagtagggtttccatccctgggtctggtaggtgaggggcctctggagtcaggaaagctggggatggaatggaagctggtctggcagcctgcccctggtgatggggtggattttagcaggctgggcagaggggtaccaggcatcacagttttgaaaagtggaagtgttagtcatgtccgactctttttgacccacagattgtagcccgccgagttcctctgtccatgggatttcccaggcaagcatactggagtgggtcgccatttctttcttcaggagatcttcccaacccagggatacaaccagggtctctggcattgcaggcagattctttatcatctgagccaccagggaaacgaatctgcccagttgtctaaatgggaactgcctaggttcctggactgggatgctggaggacaacggggcaaggagcctgttaagaattgaggggtagccccagtgggacttgacacataacagcataaatgtgtttgaggagttgagagggtggctggtctcaggtgccagggtgtctggagaggggtcctgaacaacgtgcagggggtatggaagggacccctcacccctggtgtatgtgggcctggggagggctgggcagcctgttgggagctggaagtgaaggcccgcatgggggacctgcaccgagagtctaggagtctgggggctggagaggggcctgggtggagatccctggctttccccttccagacaccaccgccaccagcaggcaaacaccctccgcctcagtttctcccacccccacggtcccttccccccacccatccagggggcggggccagaggtcaaggctagtgggtgggattggggagggagagaggtgttgagcagtctctaggagatccctcgttttcctaggcccccggctcggggtgccttccttccccatggcgggacacctcgcttctgacttcgccttctcgcccccgccgggcggtggaggcgatgggccgggagggccagagccgggctgggttgatcctcggacctggatgagcttccaagggcctcccggtgggtcggggatcgggccgggggttgtgcctggcgccgaggtgtgggggcttcccccgtgccccccgccctatgacttgtgtggagggatggcctactgtgcgccgcaggttggagtggggccggtgcccccaggcggcctggagacccctcagcccgagggcgaggcgggagccggggtggagagcaactccgagggggcctccccggacccctgcgccgcacccgcaggcgccccgaagctggacaaggagaagctggagccgaaccctgaggaggcgagtgagctgctgggggcggggggccggtgaccgcgtaggccgggggcggccacgcgaggggaggtggtcggggacccaggaagggagcaggcgaaggcggctccccggggcagggaccagtgtgtggcggctgcaggccgctctgagctggagccaggtgggaaccaggggccggaatttaagagagaaccgcaggtgagggcagggcggcctggggcggtcggaagccggccttgcttcccccctcggcccttgtcaagtaggtccttccacttcctaggtctgacctgggtcgggtcactcattaccagccagcaccagacctaccttggggtctgagccccttctacccggatccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctagatgcaaaacttcctcaaattgggttaatactagagtcaaaagcttttgtgagtactaaagaactaaaaggcaggagcccccttcctaacctgacatgtgtcctcattgaggttagagcttctctttgcattattgaatggtgctctgcatctggtggatgttgctttctgggctagatgacatttgtgttatttctggttgttaggtgggcagctttgaggcagagatactctcacataccttggggccccagtggaagagggggtgaggtggatagctgtcatatgttcttacatatcctctgccttttaaaatgcagtcccaggacatcaaagctcttcagaaagaccttgaacaatttgccaagctcctaaagcagaagaggatcacactaggatatacccaggccgatgtggggctcaccctgggggttctctttggtgagtctcctacagcatgttttgacccacaaaacacagggactacatctgccctggaatgaacatccctgaattggggcctttccctccccaccaagggagtggtggagggtggggtggaggggaagatactcgacatggacttttgagttctctgggaagagtagccggtgtggggtcttggaagggtggggacaggtggcaaggcctgtgtcggtcttgtggcaaaggggccctacctgctacctcactttgcttcttccaacccacctccccaggaaaggtgttcagccaaacgactatctgccgttttgaggctttgcagctcagtttcaagaacatgtgtaagctgcggcccctgctgcagaagtgggtggaggaagctgacaacaacgagaatctgcaggaggtgagggtgggagggaggcacagtgactgccctatctcaagtctgatttccactgctttcatctctggctggcagcgcgccctctaggagccagtggccctcaatggcatgaagtgtgattcctctgttctgctgagagaaggtccaggatcacttgtctaaacctatctttccaggatctttttcatacctcctggccctagtgacaactggctggtgctcctgaatagttggctcttgagtcattgtcccccctgatactggtcctgagcagctttggggcttgcctgcccatttctttccttgtcatctcctccccacctgctcagatatgcaaggcagagacccttgtgcaggcccgaaagagaaagcggacgagtatcgagaaccgagtgagaggcaacctggagagcatgttcctgcagtgcccgaagcccaccctgcagcaaattagccacatcgcccagcagctcgggctggagaaagacgtgagtgcacctgtccctgtccctgtgtgcgccgtctcctcctccctggtgtcacacctcccagatctcaggaccaaatgcccctcccgtgtcctccactcctaggtggtccgagtgtggttttgcaaccgtcgccagaagggcaaacgatcaagcagtgactactcccaacgtgaggattttgaggctgctgggtctcctttcacagggggacccgtatcctctcctctggcgccagggccccattttggtaccccaggctacggggggcctcacttcactactctgtactcttcggtcccattccctgagggtgaggtctttccctcggtgtctgtcaccgctctgggctcccctatgcatgcaaactgaggtgcctgatcaccccaggaatagggggcagaggaaggggagagctagggagagaaccctggggtttgtaccaggcctttgggattaagtttttcattcactaagaaaggaattgggaacacaatgggtgttggggcagggagtttggggaaactggttggagggaaggtgaagttcaatgatgctcttgactttaatccccacatcactcatcactttgttcttaaataaagaagcctgggacctagtaggtggatatttttgtctttggtttatcctctagttattgaagagtggataggatattaatagtgggtattaatagctctctgacccaaaatgaccccgaaacgaaggttgctgtttacattgcgcagagtcacaatgtcagagggaggtgtggggttggatcc

SEQ ID NO: 67 - DNA, 1083 bp, Bos taurus
  source 1..1083: mol_type = other DNA
atggcgggacacctcgcttctgacttcgccttctcgcccccgccgggcggtggaggcgatgggccgggagggccagagccgggctgggttgatcctcggacctggatgagcttccaagggcctcccggtgggtcggggatcgggccgggggttgtgcctggcgccgaggtgtgggggcttcccccgtgccccccgccctatgacttgtgtggagggatggcctactgtgcgccgcaggttggagtggggccggtgcccccaggcggcctggagacccctcagcccgagggcgaggcgggagccggggtggagagcaactccgagggggcctccccggacccctgcgccgcacccgcaggcgccccgaagctggacaaggagaagctggagccgaaccctgaggagtcccaggacatcaaagctcttcagaaagaccttgaacaatttgccaagctcctaaagcagaagaggatcacactaggatatacccaggccgatgtggggctcaccctgggggttctctttggaaaggtgttcagccaaacgactatctgccgttttgaggctttgcagctcagtttcaagaacatgtgtaagctgcggcccctgctgcagaagtgggtggaggaagctgacaacaacgagaatctgcaggagatatgcaaggcagagacccttgtgcaggcccgaaagagaaagcggacgagtatcgagaaccgagtgagaggcaacctggagagcatgttcctgcagtgcccgaagcccaccctgcagcaaattagccacatcgcccagcagctcgggctggagaaagacgtggtccgagtgtggttttgcaaccgtcgccagaagggcaaacgatcaagcagtgactactcccaacgtgaggattttgaggctgctgggtctcctttcacagggggacccgtatcctttcctctggcgccagggccccattttggtaccccaggctacgggggccctcacttcactactctgtactcttcggtcccattccctgagggtgaggcctttccctcggtgtctgtcaccgctctgggctctcctatgcattcaaactga

SEQ ID NO: 68 - DNA, 1644 bp, Bos taurus; identical to SEQ ID NO: 60
  source 1..1644: mol_type = other DNA

SEQ ID NO: 69 - DNA, 1083 bp, Sus scrofa
  source 1..1083: mol_type = other DNA
atggcgggacacctggcttccgacttcgccttctcgcccccgccgggcggtggaggcgatgggccgggagggccggagccgggctgggttgatcctcggacctggctgagcttccaagggcctcccggtgggtcagggatcgggccgggggttgggccgggcgccgaggtgtgggggcttcccgcgtgccccccgccctatgacttctgcggagggatggcctactgcgcacctcaggtcggagtggggctggtgccccagggcggcctggagacccctcagcccgagggcgaggcgggggccggggtggagagcaactccgagggggcctcccccgagccctgtgccgcccccgctggcgccgcgaagctggacaaggagaagctggagccgaaccccgaggagtcccaggacatcaaagcgcttcagaaagatctcgaacaatttgccaagctcctaaagcagaagaggatcaccctgggatatacccaggccgatgtggggctcactttgggggttctctttgggaaggtgttcagccaaacgaccatctgccgttttgaggctttgcagctcagtttcaagaacatgtgtaagctgcggcccctgctgcagaagtgggtggaggaagctgacaacaacgagaatctgcaggagatatgcaaggcagagaccctcgtgcaggcccggaagagaaagcggacaagtatcgagaaccgagtgagaggcaacctggagagcatgttcctgcagtgcccaaagcccactctgcagcagatcagccacatcgcccagcagctcgggctagagaaggatgtggtccgcgtgtggttctgcaaccgtcgccagaagggcaaacgatcaagcagtgactattcgcaacgagaggattttgaggctgctgggtctcctttcccagggggaccagtatcctttcctctggcgccagggccccattttggtaccccaggctatgggggccctcacttcaccaccctgtactcctcggtcccattccctgagggtgaggcctttccctcggtgtctgtcacccctctgggctcccccatgcattcaaactga

SEQ ID NO: 70 - DNA, 1360 bp, Sus scrofa
  source 1..1360: mol_type = other DNA
cccctagcctgggaacttccatatgctgcaggtgcggccctaaaaagcaaaaagaagaaaaagaaaaaagagaatcagagaggtgctctctatttctctagtctcaaaacttcctcaaactggtaggtaatactaaagtcaaaacgcttttgtgagtattaaagaactaaagggcaggagcctttttaatgacctgacaggtgttctcactgaggctggggcctctttccactgaatggtcttctgcatttgataggtgtgtcactttctggagtaaatgaccttcaaataaaggttatttctggttgttaggtgggtagcctacagatacccccacagaccatttactccttggggcctaagtggaaagggggtggggtgggtaagtggtacgtttatgttcctacatgtcctctgcctttttaaatgcagtcccaggacatcaaagcgcttcagaaagatctcgaacaatttgccaagctcctaaagcagaagaggatcaccctgggatatacccaggccgatgtggggctcactttgggggttctctttgggaaggtgttcagccaaacgaccatctgccgttttgaggctttgcagctcagtttcaagaacatgtgtaagctgcggcccctgctgcagaagtgggtggaggaagctgacaacaacgagaatctgcaggagatatgcaaggcagagaccctcgtgcaggcccggaagagaaagcggacaagtatcgagaaccgagtgagaggcaacctggagagcatgttcctgcagtgcccaaagcccactctgcagcagatcagccacatcgcccagcagctcgggctagagaaggatgtggtccgcgtgtggttctgcaaccgtcgccagaagggcaaacgatcaagcagtgactattcgcaacgagaggattttgaggctgctgggtctcctttcccagggggaccagtatcctttcctctggcgccagggccccattttggtaccccaggctatgggggccctcacttcaccaccctgtactcctcggtcccattccctgagggtgaggcctttccctcggtgtctgtcacccctctgggctcccccatgcattcaaactgaggtgcctgcccttcccaggagtgggggggggtgaggaaggggtgagctagggagagaagcctggggtttgtaccagggctttgggattaagttcttcattcactaagaaaggaattgggaacacaaagggtgtgggggcagggagtctaggggaactggttggagggaaggtgaagttcaatgatgctcttgattttaatccccacatcactcatcactttgttcttaaataaagaagcctgggacccag

SEQ ID NO: 71 - DNA, 1671 bp, Sus scrofa
  source 1..1671: mol_type = other DNA
cccctagcctgggaacttccatatgctgcaggtgcggccctaaaaagcaaaaagaagaaaaagaaaaaagagaatcagagaggtgctctctatttctctagtctcaaaacttcctcaaactggtaggtaatactaaagtcaaaacgcttttgtgagtattaaagaactaaagggcaggagcctttttaatgacctgacaggtgttctcactgaggctggggcctctttccactgaatggtcttctgcatttgataggtgtgtcactttctggagtaaatgaccttcaaataaaggttatttctggttgttaggtgggtagcctacagatacccccacagaccatttactccttggggcctaagtggaaagggggtggggtgggtaagtggtacgtttatgttcctacatgtcctctgcctttttaaatgcagtcccaggacatcaaagcgcttcagaaagatctcgaacaatttgccaagctcctaaagcagaagaggatcaccctgggatatacccaggccgatgtggggctcactttgggggttctctttggtgagtcttcctcagtatgttctgacccacagaacacagggccacacgtgtccctagaaggaacatccccaaatgtgggcctttcctccccaccaagggagtgaggggagggggtttccaccctctttatgggtgagggcaggggggagaaagataccgtactttgctttggtgcttggccttgcattctctgggaggagttcctgccatggggtcccagggagggcggaagaggtggcagggcttgagtcttggggtgcggagggccctgcctgctgcctcaccttgcttctttccacccacctccccagggaaggtgttcagccaaacgaccatctgccgttttgaggctttgcagctcagtttcaagaacatgtgtaagctgcggcccctgctgcagaagtgggtggaggaagctgacaacaacgagaatctgcaggagatatgcaaggcagagaccctcgtgcaggcccggaagagaaagcggacaagtatcgagaaccgagtgagaggcaacctggagagcatgttcctgcagtgcccaaagcccactctgcagcagatcagccacatcgcccagcagctcgggctagagaaggatgtggtccgcgtgtggttctgcaaccgtcgccagaagggcaaacgatcaagcagtgactattcgcaacgagaggattttgaggctgctgggtctcctttcccagggggaccagtatcctttcctctggcgccagggccccattttggtaccccaggctatgggggccctcacttcaccaccctgtactcctcggtcccattccctgagggtgaggcctttccctcggtgtctgtcacccctctgggctcccccatgcattcaaactgaggtgcctgcccttcccaggagtgggggggggtgaggaaggggtgagctagggagagaagcctggggtttgtaccagggctttgggattaagttcttcattcactaagaaaggaattgggaacacaaagggtgtgggggcagggagtctaggggaactggttggagggaaggtgaagttcaatgatgctcttgattttaatccccacatcactcatcactttgttcttaaataaagaagcctgggacccag

SEQ ID NO: 72 - DNA, 1217 bp, Ovis aries
  source 1..1217: mol_type = other DNA
cccatggcgggacacctcgcttctgacttcgccttctcgccgccgccggggggtggaggcgatgggccgggagggccagagccgggctgggttgatcctcggacctggatgagcttccaagggcctcctggtgggtcagggatcggaccgggggttgtgcccggcgccgaggtgtgggggcttcccccgtgccccccgccctatgacttgtgcggagggatggcctactgtgcgccacaggttggagtggggccggtgcccccaggcggcctggagacccctcagcccgagggcgaggcgggagccggggtggagagcaactccgagggggcctccccggacccctgtgccgcccccgcaggcgccgcgaagctggacaaggagaagctggagccgaaccctgaggagtcccaggacatcaaagctcttcagaaagaccttgaacaatttgccaagctcctaaagcagaagaggatcaccctaggatatactcaggccgatgtggggctcactctgggggttctctttggaaaggtgttcagccaaacgactatctgccgttttgaggctttgcagctcagtttcaagaacatgtgtaagctgcggcccctgctgcagaaggtggaggaagctgacaacaatgagaatctgcaggagatatgcaaggcagagacccttgtgcaggcccgaaagagaaagcggacgagtatcgagaaccgagtgagaggcaacctggagagcatgttcctgcagtgcccgaagcccaccctgcagcaaattagccacatcgcccagcagctcgggctggagaaggacgtggtcagagtgtggttctgcaaccgtcgccagaagggcaaacgatcaagcagtgactactcccaacgcgtggattttgaggctgcgggggctcctttcgcagggggacccgtatcctttcctctgaagccagggccccattttggtaccccaggctaccgggaccctcatttcactacactgtactcttcggtcccatttcctgagggtgaggcctttccctcggtgtctgtccccgctctgggttctcctatgcattcaaactgaggtgcctgctcaccccaggaatggggggcagaggaaggggagagctagagagagaaccctggggtttgtactagggctttgggattaagtttttcattcactaagaaaggaattgggaacacaatgggtgtggg

SEQ ID NO: 73 - DNA, 1591 bp, Ovis aries
  source 1..1591: mol_type = other DNA
gagtctggggcctggggaggcgcctgggtggagatccctggctttccccttccagacaccactgccaccagcaggcaaacaccctccgcctcagtttctcccacccccaccgtcccttccccccacccatccagggggcggggccagaggtcaaggctagtgggtgggattggggagggagagaggtgttgagcagtctctaggagatccctcgttttcctaggcccccggctcggggtgccttccttccccatggcgggacacctcgcttctgacttcgccttttcgcccccgccgggcggtggaggcgatgggccgggagggccagagccgggctgggttgatcctcggacctggatgagcttccaagggcctcctggtgggtcggggatcggaccgggggttgtgcccggcgccgaggtgtgggggcttcccccgtgccccccgccctatgacttgtgcggagggatggcctactgtgcgccgcaggttggagtggggcccgtgcccccaggcggcctggagacccctcagcccgagggcgaggcgggagccggggtggagagcaactccgagggggcctccccggacccctgtgccgcccccgcaggcgccgcgaagctggacaaggagaagctggagccgaaccctgaggagtcccaggacatcaaagctcttcagaaagaccttgaacaatttgccaagctcctaaagcagaagaggatcaccctaggatatacccaggccgatgtggggctcaccctgggggttctctttggaaaggtgttcagccaaacgactatctgccgttttgaggctttgcagctcagtttcaagaacatgtgtaagctgcggcccctgctgcagaagtgggtggaggaagctgacaacaacgagaatctgcaggagatatgcaaggcagagacccttgtgcaggcccgaaagagaaagcggacgagtatcgagaaccgagtgagaggcaacctggagagcatgttcctgcagtgcccgaagcccaccctgcagcaaatcagccacatcgcccagcagctcgggctggagaaggacgtggtccgagtgtggttctgcaaccgtcgccagaagggcaaacgatcaagcagtgactactcccaacgcgaggattttgaggctgctgggtctcctttcgcagggggacccgtatcctttcctctggcgccagggcccccttttggcaccccaggctacgggggccctcacttcactacgctgtactcttcggtcccattccctgagggtgaggcctttccctcggtgtctgtcaccgctctgggttctcctatgcattcaaactgaggtgcctgctcaccccaggaatggggggcagaggaaggggagagctagggagagaaccctggggtttgtactagggctttgggattaagtttttcattcactaagaaaggaattgggaacacaatgggtgtgggggcagggagtttggggaaactggttggagggaaggtgaagttcaatgatgctcttgactttaatccccacatcactcatcactttgttcttaaataaagaagcctgggacctagtaggtgga

SEQ ID NO: 74 - DNA, 1527 bp, Ovis aries
  source 1..1527: mol_type = other DNA
gggaagaaacgctgttcgtgggttaaaaacaaaaagcagaatgaaggaatttatgggcaggttgagagaggagcgagctgcatttaggtctctggttctcaaactgctcaacaattaacatctgcgggctgtgaaaacgcagatctgcccgggtccgcggaaattcccagtcgtaggtccgaagggagatcccggaacccgacttatttcaagcaccctctgggtcgattctgaatagggagatacttttccgaggcccccgagcccacggggcctctcttgaattctaggatccaagtacattccctaccgcctcgcgtcccctctggaaggaaagcgccagccgcggagctggccttcgaatcccgtccacccaccccagaggaaagcaaaaacaggctccagggaaacatacacactagacccagtgtgactttcttcaaagaccagggtgacttccggaagtgatataatagctgcagagccgtgcagatgctgccaaaatgcccgagtgagctctgacttgggctttggtcctggaccagccccagatgagtgacctgtgattgaaaatggagggataaactgtaaagtcccaggacatcaaagctcttcagaaagaccttgaacaatttgccaagctcctaaagcagaagaggatcaccctaggatatacccaggccgatgtggggctcaccctgggggttctctttggaaaggtgttcagccaaacgactatctgccgttttgaggctttgcagctcagtttcaagaacatgtgtaagctgcggcccctgctgcagaagtgggtggaggaagctgacaacaacgagaatctgcaggagatatgcaaggcagagacccttgtgcaggcccgaaagagaaagcggacgagtatcgagaaccgagtgagaggcaacctggagagcatgttcctgcagtgcccgaagcccaccctgcagcaaatcagccacatcgcccagcagctcgggctggagaaggacgtggtccgagtgtggttctgcaaccgtcgccagaagggcaaacgatcaagcagtgactactcccaacgcgaggattttgaggctgctgggtctcctttcgcagggggacccgtatcctttcctctggcgccagggcccccttttggcaccccaggctacgggggccctcacttcactacgctgtactcttcggtcccattccctgagggtgaggcctttccctcggtgtctgtcaccgctctgggttctcctatgcattcaaactgaggtgcctgctcaccccaggaatggggggcagaggaaggggagagctagggagagaaccctggggtttgtactagggctttgggattaagtttttcattcactaagaaaggaattgggaacacaatgggtgtgggggcagggagtttggggaaactggttggagggaaggtgaagttcaatgatgctcttgactttaatccccacatcactcatcactttgttcttaaataaagaagcctgggacctagtaggtgga

SEQ ID NO: 75 - DNA, 1476 bp, Bos taurus
  source 1..1476: mol_type = other DNA
gtcaaggcagagaagagagtgtttgcaaaagggggaaagtagtttgctgcctctttaagactaggactgagagaaagaagaagagagagaaagaaagggagagaagtttgagccccaggcttaagcctttccaaaaaataataataacaatcatcggcggcggctggatcggccagaagaggagggaagcgcttttttgatcctgattccagtttgcctctttcttttcttcccccaaattattcttcgcctgattttcctcgcggagccctgcgctcccgacacccccgcccgcctcccctcctcctctcccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcggcggcggcaactccaccgcggcggcggcgggcggcaaccagaagaacagcccggaccgagtcaagcggcccatgaacgccttcatggtgtggtcccgcgggcagcggcgcaagatggcccaagagaaccctaagatgcacaactcggagatcagcaagcgcttgggcgccgagtggaaacttttgtccgagacggagaagcggccgttcatcgacgaggccaagcggctgcgagcgctgcacatgaaggaacacccggattataaataccggccccggcggaaaaccaagacgctcatgaagaaggataagtacacactgccgggagggctgctcgccccgggcggcaacagcatggcgagcggggtcggggtgggcgccggcctcggcgcgggcgtgaaccagcgcatggacagctacgcgcacatgaacggctggagcaacggcagctacagcatgatgcaggaccagctgggctacccgcaacacccgggcctcaacgcgcacggcgccgctcagatgcagcccatgcaccgctacgacgtgagcgccctgcagtacaactctatgaccagctcgcagacctacatgaacggctcgcccacctacagcatgtcctattctcagcagggcacccctggcatggcgcttggctccatgggctcggtggtgaagtccgaggccagctccagcccccccgtggttacctcttcttcccactccagggcgccctgccaagccggggacctccgggacatgatcagcatgtacctccccggcgccgaggtgccggagcccgccgcccccagcagacttcacatgtcccagcactaccagagcggcccggtgcccggcacggccattaacggcacactgcccctctcgcacatgtgagggccggacggtgaactggagggggcgggggagacattttcaaagaaaaagagggaaatggaggacagcaagaaacagcatggagaaaaacccggtacgctcaaaaaaaaaaaaaaaa

SEQ ID NO: 76 - DNA, 1477 bp, Bos taurus
  source 1..1477: mol_type = other DNA
ggtcaaggcagagaagagagtgtttgcaaaagggggaaagtagtttgctgcctctttaagactaggactgagagaaagaagaagagagagaaagaaagggagagaagtttgagccccaggcttaagcctttccaaaaaataataataacaatcatcggcggcggctggatcggccagaagaggagggaagcgcttttttgatcctgattccagtttgcctctttcttttcttcccccaaattattcttcgcctgattttcctcgcggagccctgcgctcccgacacccccgcccgcctcccctcctcctctcccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcggcggcggcaactccaccgcggcggcggcgggcggcaaccagaagaacagcccggaccgagtcaagcggcccatgaacgccttcatggtgtggtcccgcgggcagcggcgcaagatggcccaagagaaccctaagatgcacaactcggagatcagcaagcgcttgggcgccgagtggaaacttttgtccgagacggagaagcggccgttcatcgacgaggccaagcggctgcgagcgctgcacatgaaggaacacccggattataaataccggccccggcggaaaaccaagacgctcatgaagaaggataagtacacactgccgggagggctgctcgccccgggcggcaacagcatggcgagcggggtcggggtgggcgccggcctcggcgcgggcgtgaaccagcgcatggacagctacgcgcacatgaacggctggagcaacggcagctacagcatgatgcaggaccagctgggctacccgcaacacccgggcctcaacgcgcacggcgccgctcagatgcagcccatgcaccgctacgacgtgagcgccctgcagtacaactctatgaccagctcgcagacctacatgaacggctcgcccacctacagcatgtcctattctcagcagggcacccctggcatggcgcttggctccatgggctcggtggtgaagtccgaggccagctccagcccccccgtggttacctcttcttcccactccagggcgccctgccaagccggggacctccgggacatgatcagcatgtacctccccggcgccgaggtgccggagcccgccgcccccagcagacttcacatgtcccagcactaccagagcggcccggtgcccggcacggccattaacggcacactgcccctctcgcacatgtgagggccggacggtgaactggagggggcgggggagacattttcaaagaaaaagagggaaatggaggacagcaagaaacagcatggagaaaaacccggtacgctcaaaaaaaaaaaaaaaa

SEQ ID NO: 77 - DNA, 960 bp, Bos taurus
  source 1..960: mol_type = other DNA
atgtacaacatgatggagacggagctgaagccgcccggcccgcagcaaacttcagggggcggcggcggcggtggcaactctactgctgcggcggcgggcggcaaccagaagaacagcccagaccgagttaagcggcccatgaacgctttcatggtgtggtcccgagggcagcggcgcaagatggcccaagagaaccccaagatgcacaactcggagatcagcagacggctgggcgccgagtggaaacttttgtcggagacggagaagcggccgttcatcgacgaggccaagcggctgcgagcgctgcacatgaaggagcacccggattataaataccggccccggcggaaaaccaagacgctcatgaagaaggataagtacacactgcccggagggctgctggccccgggaggcaacagcatggcgagcggggtcggggtgggcgccggcctcggcgcgggcgtgaaccagcgcatggacagctacgcgcacatgaatggctggagcaactgcagctacagcatgatgcaggaccagctgggctatccgcagcacccgggcctcaatgcgcacagcgcggctcagatgcagcccatgcaccgctacgacgtgagcgccctgcagtacaactccatgaccagctcgcagacctacatgaacggctcgcccacctacagcatgtcctactcgcagcagggcacccctggcatgacgctcggttccatgggctcagtggtcaagtccgaggcgagctccagcccccccgtggttacctcttcttcccactccagggcgccctgccaggccggggacctacgggacatgatcagcatgtacctccccggcgctgaggtgccagagcccgccgcccccagcagacttcacatgtcccagcactaccagagcggcccggtgcccggcacggccatcaacggtacactgcctctctctcacatgtga

SEQ ID NO: 78 - DNA, 960 bp, Sus scrofa; identical to SEQ ID NO: 77
  source 1..960: mol_type = other DNA

SEQ ID NO: 79 - DNA, 1036 bp, Ovis aries
  source 1..1036: mol_type = other DNA
atgtacaacatgatggagacggagctgaagccgccgggcccgcagcaaacttcggggggcggcggcggcggcggcggcaactccaccgcggcggcggcgggcggcaaccagaagaacagcccggaccgagtcaagcggcccatgaacgccttcatggtgtggtcccgcgggcagcggcgcaagatggcccaagagaatcctaagatgcacaactcggagatcagcaagcgcttgggcgccgagtggaaacttttgtccgagacagagaagcggccgttcatcgacgaggccaagcggctgcgagcgctgcacatgaaggagcacccggattataaataccggccccggcggaaaaccaagacgctcatgaagaaggataagtacacactgccgggagggctgctcgccccgggcggcaacagcatggcgagcggggtcggggtgggcgccggcctcggcgcgggcgtgaaccagcgcatggacagctacgcgcacatgaacggctggagcaacggcagctacagcatgatgcaggaccagctgggctacccgcagcacccgggcctcaacgcgcacggcgccgctcagatgcagcccatgcaccgctacgacgtgagcgccctgcagtacaactctatgaccagctcgcagacctacatgaacggctcgcccacctacagcatgtcctattctcagcagggcacccctggcatggcgcttggctccatgggctcggtggtgaagtccgaggccagctccagcccccctgtggttacctcttcttcccactccagggcgccctgccaagccggggacctccgggacatgatcagcatgtacctccccggcgccgaggtgccggagcccgccgcccccagcagacttcacatgtcccagcactaccagagcggcccggtgcccggcacggccattaacggcacactgcccctctcgcacatgtgagggccggacggtgaactggaaggggcgggggagacattttcaaagaaaaagagggaaatggaggacagcaaga

SEQ ID NO: 80 - DNA, 1036 bp, Ovis aries; identical to SEQ ID NO: 79
  source 1..1036: mol_type = other DNA

SEQ ID NO: 81 - DNA, 3054 bp, Ovis aries
  source 1..3054: mol_type = other DNA
aaaaaataataataacaatcatcggcggcggctggatcggccagaagaggggaagcgcttttgatcctgattccagtttgcctctctctttcttttcttccccaaattatcttcgcctgattttcctacgcggagccctgcgctcccggcacccccgcccgcctcccctcccctctcccccccgcccgcgggccccccaaagtcccggccgggccgagggtcggcggccgccggcgggccgggcccgcgcacagcacccgcatgtacaacatgatggagacggagctgaagccgccgggcccgcagcaaacttcggggggcggcggcggcggcggcggcaactccaccgcggcggcggcgggcggcaaccagaagaacagcccggaccgagtcaagcggcccatgaacgccttcatggtgtggtcccgcgggcagcggcgcaagatggcccaagagaatcctaagatgcacaactcggagatcagcaagcgcttgggcgccgagtggaaacttttgtccgagacagagaagcggccgttcatcgacgaggccaagcggctgcgagcgctgcacatgaaggagcacccggattataaataccggccccggcggaaaaccaagacgctcatgaagaaggataagtacacactgccgggagggctgctcgccccgggcggcaacagcatggcgagcggggtcggggtgggcgccggcctcggcgcgggcgtgaaccagcgcatggacagctacgcgcacatgaacggctggagcaacggcagctacagcatgatgcaggaccagctgggctacccgcagcacccgggcctcaacgcgcacggcgccgctcagatgcagcccatgcaccgctacgacgtgagcgccctgcagtacaactctatgaccagctcgcagacctacatgaacggctcgcccacctacagcatgtcctattctcagcagggcacccctggcatggcgcttggctccatgggctcggtggtgaagtccgaggccagctccagcccccctgtggttacctcttcttcccactccagggcgccctgccaagccggggacctccgggacatgatcagcatgtacctccccggcgccgaggtgccggagcccgccgcccccagcagacttcacatgtcccagcactaccagagcggcccggtgcccggcacggccattaacggcacactgcccctctcgcacatgtgagggccggacggtgaactggaaggggcgggggagacattttcaaagaaaaagagggaaatggaggacagcaagaaacagcatggagaaaaacccggtacgctcaaaaaaaaaaaaaaaaatcatccacagcaaatgacagctgcaaaagaaaacgccagtcccatctacactcacgcaaaaaccgcgatgccggcaagaaaacttttatgagagatcctggacttcttttggggggactatttttgtacagagaaaacctggggtgggcaggtggggagggcgagggaatggaccttgtatagatctggaggaaagaaaactacgcaaaactttttaaaagttctagtgatacggtaggagctttgcagaaagtttgcaaaagtctttaccaataatatttagagctagtctccaagcgacgaaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgcaaagaagaaaattaatcagattgggcattttaattgtttaaaaattgtacaaaaggaaaaaaattagaataagtactggcaaaaccatctccgtggtctctaaaggggcaaaagttttagattgtactaaatttttttaacttaactgttaaaagcaaaaatggccatgcaggttgacatcgttggtaatttataatagcttttttgtttcccaactttccattttgttcagataaaaaaccaacatgaaattactgtgtttgaaatattttcttatggtttgtaatatttctgtaaatttattgtgatattttaaggttttcccacttttagtttccgtagttgtattttaaagattcggctctgtattattgaatcagtctgccgagaatccatgtatatatttgaactaataccatccttataacaggtacattttcaacttaagtttttactccattatgcacagtttgagataaataaatttttgaaatatggacactgaaattatgcttgagtctttcatttatttggataacattgtgattataacccagctcctcggggaatatactgtgctcagccaagaacggcagagtacctaaaaccttggtattctagtccagtagcatgtgctaatgttaacagcaacaaataaatggctggatgattaattgaataagtactctcgaaaaaatggaaatgcttatgtcatttccaaaacagaaactagggacatatattgtaaacccaccccctccttttctaagcagcatgaataagcatgatggggacttaaactgatttatgccacttgttgccagaaaggagtcaattctgaaaaatttcatgtagcttagcagatggggtattgagccaaatcagaaccaggttggctggttttgttttcgttttcttcacagcacaatggccctttggaaacgtggttatagggggaaatgatgcttcttgtataggaataattgcaaactagacaagcaaagtgctcatctgctgggtgggtgtttagattactctggccctgctattgtctgagaagaaacaagtggtttgcttctttgttccatcacttcataaactttttctctacattttcctgtttaggggcgagggaaagcagagcgcggcttgcaaaaactccccgacttatcacagctatttattattattattattatttattttcttttttcctctctctccctccacgaattc

SEQ ID NO: 82 - DNA, 3388 bp, Bos taurus
  source 1..3388: mol_type = other DNA
atgtcgaggcgcaagcaggcgaagccccagcacatcaactcggaggaggatcagggcgagcggcagccgcagcagccggcccccgagtgtgcagatgcggccccagcggcgccggcggcgggggcgccgggtgctccaatgaactatcttggtggccgcggcaacgacgagctgaatggggacggagcagggataaaacggccccggcgggaggagacccacgtttgcgagaaatgctgtgccgagttcttcagcttctcggagttcttggaccacaagaaaaattgcactaaaacgccccccgtgctcatcttgaaggacagtgaggggccgatgtcatccgaagacttgtcagggactgtgctcagcccgcagccgccgagcccgggccggagggagggccaccgggacaatggcagcggcggaggggcccccggggacttgagggaaaagccgggggcggaatccgtcctgtatctgaagacggaggccgccctgccgcccgcgccgcaggacataagctacttacccaaaggcaaggtggccaacaccaatgtcacgcttcaggcgctgcgcggcaccaaggtggcggtgaaccagcgcagcgccgacgtgcctccggcgcccgtgcccggcgccggcagcctgccctgggtcctcgagcagatcctgtgcctccagcagcagcagctgcagcagatccagctcacggagcagatccgggtgcaggtgaacatgtgggcctcgcacgccctccacgccgggcacgcggccgactcgctgaagacgctcgggggccacgtgtcgcagcaggtgtcggcggccgtggccctgctcagccagaaggcggggagcgccgggctgcccctggacgctctgaagcccgccaagctacctcacgccaacgtcccttccgccggcggctccgcctccccggggctgccgcccttcgcgctgaagccggacgccacccgggtgctccccggcgtcctgtcgcgcctccctggggcgctgctcccccaggcccagggctctgtgctcttccagagccccttctccgcggtggccttagacccgtccaagaaaggcaaagggaagccaccgagtgtctccccggtggaggtcaaactcaaggacgaggctctctgccggcacaagtgtaagtactgtagcaaggtttttgggactgatagctccttgcagatccacctccgctcccacaccggagagagacccttcgtgtgctccgtgtgcggccaccgcttcaccaccaagggcaacctcaaggtgcacttccaccggcatccccaggtgaaggcaaacccccagctgtttgccgacttccacgacaagacggcggtgggcggcggccttcccttcgcgctggccggccccgtccccctcgaggaagccgggctctccctagacagcaaacccgcgctcgcggcggggacccccagtgtcgtagggctagctcagaatctcccctcagggcctcaccccaaggacgtcgcggggggcccgttgcccggcgacctgcagcccgggccgtctccggaaagcgaggatggctccatcctgcccggggtggggccgacccatccctcccccagggtcggaggcttcccagggggtgggccgccggagccggggtcggagaccctgaagctgcagcggctggtggagaacatagacaaggccaccaccgaccccaacgagtgtctcatctgccaccgcgtcctcagctgccagagctcactcaagatgcactaccgcacgcacaccggggagaggcctttcccgtgcaaggtctgcggccgcgccttctccaccaaaggcaacctgaagacgcacctgggggtgcaccgcaccagcgcgtcggtgaagacgcagcactcctgcccgatctgccagaagaagttcaccaacgcggttctgctgcagcagcacatccgcatgcacatgggcggccagatccccaacacgcctctgcccgagagcccctgcgagttcgcgggccccgagccctcggcggctggggagaatggcagccccggcgcgcccgctcacgatggggtcatggaagccatcgacgtagacgaagcctgcccccaggaggccgcccccagcagctcctcgagggtcctcgggcccctcgccggcgtcctcgcggcgtcccctaccccggggctggccacggcggcttcgctggacgccccggtgaaggcggggctcgctgcgctcgtcctgcagcggcagggcagccgagaaaacgggtccgtggagagtgacggcctgaccaatgacgacgcctcctcggtgatgggcgaccccgagtgcgccagccgaagcccagacgtcctagagagcacgtccttccaggcggtctcgccggcccatagccaggcggagagcgtcaagtccaagtctccagacgccgacggcaaaggggacggctccgagagcagccgcactgagatggaaggtcggagcagcgttccatccacatttatccgagcccagccaacctatgtcaaagttgaagttcccggtgggtttgtgggtcccacgaccatgtccccaggtatgacgcctctgttggcggcccagccccgacgacaggccaagcagcacggctgcacgaggtgcgggaagaacttctcgtcggccagcgcgctgcagatccacgagcggactcacaccggtgagaagccctttgtgtgcaacatctgtgggcgcgctttcaccaccaaaggcaacttgaaggtccattacatgacacatggggccaacaatagctcggcacgccgtggcaggaagctggccatcgagaacaccatggctctgttaggaacggacgcaaagagggtccccgagatgtttcccaaggagatcgtggccccttcggtgagcgtggaccccgtcgtgtggagccagtacgccaccatgctcaacggtggtctggccatgaagaccaacgagatctccgtaattcagagtggtggcgtccccaccctcccggtgtccctgggggccagctccgtggtgaataacacggccgcctccaagatcgacggctcccagtctgccgccggggccgaggtggagaagccagggacggccgacaacgtccccaaacaccagttcccgcacctcctggaagaaaacaagatcgcagtcagctaagccgcgggaggacgcacctccgaggcgcagtgcacgcagcgacctctctagagaactgtgtcttttattgttctctctctttttttaaggcccccgtctcctccagttttcttcctgatgtgcaaataacgtttacagttgtggccacaacctcaggcacggcttacaatcacaatgttgctatgctgctttgcaaaaaaag

SEQ ID NO: 83 - DNA, 3153 bp, Sus scrofa
  source 1..3153: mol_type = other DNA
atgtcgaggcgcaagcaggcgaaaccccaacacatcaactcggaggaggaccagggcgagcagcagccgcagcagcccgccgcggagtttgcagatgcggccccatcggctccggtggccggggagccgggtgcgccaatgaaccgcccggggactggtgacgaggtgagcgcggacgcagcggggataaagaggcctcggcgagaggagacccacatctgtgagaaatgctgtgccgagttcttcagtttctccgagttcttagaccataagaagaattgcactaagactccccctgtcctcatcatgaatgacagcgaggggccggtgccttcggaagacttccccggggctgtgctgagccagcagccacacagcccaagcaggaaggacactcacagcgaggaggggggcggctccggggacacgaaggagaagccggggaccgagtccgttgtgtacttgaagacggaggctgccctgccgcccacaccccaggacataagctatttacccaaaggcaaagtggccaacaccaatgtcacgcttcaagcactacggggcaccaaggtggctgtgaaccagcggagtgcggacgcgccgccggcccccctgcccggcgccaacagcatcccctgggttctggagcagatcctgtgcctgcagcagcagcagctccagcagatccagctcaccgagcagatccgtgtccaggtgaacatgtgggcctcgcacgccctccactcgggcgtggccggcgccgatgccctcaagacgctgggcagccacgtgtcccagcaggtgtcggccgccgtggctctgctcagccagaaagcgggaagcccgggtctgtctctggaccccttgaaaccagccaagctacctcacgctaacatccctcccgccgccagctccgtgtccccagggctgacgcccttcgccctgaagccggatgggacccgggtgctccccggtgtgctgtcgcgcctcccgggtgcattgctacctcaggccccgggctctgtgctcttccagagccccttctccgcgggggcgttagacccgtccaggaaaggcaaagggaagcccccgagtgtgtccccggtggatggcaaacccaaggacgaggccctctacaagcacaagtgtaagtactgtagcaaggtttttgggactgatagctccttgcagatccacctccgctcccataccggagagagaccctttgtgtgctctgtctgcggccaccggttcaccaccaagggcaacctcaaggtgcactttcatcgacacccccaggtgaaggcgaacccccagctttttgctgacttccacgacaagatgacggcaggcaacggcattccctgtgcactctctggccccgtccccgtagatgaacccggtctctctttagacagcaaacctgtccttgcgacggggaccccccatgtagggctacctcagaatctctcttcggggactcaccccaaggacctcaagcaagacccgcagcccaggccttctccagaaaatgagaatggacccatcctgcccggaggggggccgaaccataattcccccagggttggtggcttccaagggagtggggcacccgagccggggtccgagaccctgaaactgcagcagctggtggagaacatcgacaaggctcccaccgaccccaacgaatgtctcatttgccaccgtgtcttgagctgccaaagctccctcaaaatgcattaccgcacccacaccggggagaggccgttccagtgtaagatctgtggtcgcgccttctccaccaaaggcaacctgaagacgcacctgggggtccaccggaccaacacgtccataaagacacagcattcgtgccccatctgccagaagaagtttaccaacgccgtcctgttgcagcagcatatccggatgcacatgggcggccagattcccaacacgcctctgcccgagaatccctgtgactttgcgggtcctgagccgatgatggtcagtgaaaacggcagtaccggcgccgtctgtctggatgacgtggaaagcatcgacgtggatgacgtctgcgcccaggaggcccccagcagctcctccaaggtcgccgtgcctcttcccggcgtccactcggcgtcccccactctggggttgaccatgatggcttccctagatgccccgggaaaggggggactcactgcccttgtcctgcagcgacagagcagccgagaaaacggctcagtggagagcgatggcttgaccaatgactcttcatcctcagtgatgggagaccaggagtaccagagccgaagtccagatgtcctggagaccacctccttccaggcagtctctccggccaatagccaagccgagagcatcaagtccaagtctcccgaggttggcagcaaagcggacagctccgagaacagccgcactgagatggaaggtcgaagcattccatcgacgtttatccgagcccagcccaccacctacgtcaaagttgaagttcctggtgcatttgttggtcctgcgaccatgtccccaggtatgacaccgttgttagcggcccaaccccgacgacaggccaagcagcacggctgcacgaggtgcgggaagaacttctcatctgccagtgctctgcagattcatgagcgcactcacactggggagaagccttttgtgtgtaatgtatgcgggcgagcctttaccaccaaaggcaacttgaaggtccattacatgacgcatggggccaacaacagctcggcgcgccgcgggcggaagctggccatcgagaacaccattgctctgttagggacggacggaaagagagtccccgagatgttccccaaggaaatcctggccccttcggtgaacgtggaccctgtggtgtggaaccagtacaccaccctactcaacggtgggctggccatgaagaccaatgaaatctccgtgatccagagcggtggcattcccaccctcccggtgtccctgggggccagctccgtggccgctaacaccgcgctctccaagatggatggttcgcagtcagcgatcagcgccgaggccgagaagcccggcgccgccgacgggggcgccgccaaacaccagtttcctcacttcctggaagaaaacaagatcgcggtcagctga

SEQ ID NO: 84 - DNA, 5070 bp, Ovis aries
  source 1..5070: mol_type = other DNA
cgcccctccctgatcaatgaggacttatttaaatgatctctgaggtcttggacccaggccaatcagctgtcagggctcatgataaatcgcaatgcattattgataataataattactgggacatgcgcgttccggccgaaggggggtaaatttcccaactccaggaatttgtggcggagtgggcaaataaccgcggctctccaggcgccccgatgctcgcaccatgtcgaggcgcaagcaggcgaagccccagcacatcaactcggaggaggatcagggcgagcggcagccgcagcagccggcccccgagtgtgcagatgcggccccagcggcgccggcggcgggggcgccgggtgctccagtgaactatcttggcggcggcggcagcagtggcagcgaggagccgaatggcgccggagcagggataaagcggccccggcgggaggagacccacgtctgcgagaaatgctgtgcggagttcttcagcttctccgacttcttggaccacaagaaaaattgcactaaaacgccccccgtgctcatcctgaaggacagtgaggggccgatgtcgtccgaagacttgccggggactgtgctcagctcgcagccgccgagcccgggccggagggagggccaccgggacgatggcagcggcggaggggcccccggggacgcgagggagaagccaggggcggagtctgtcctgtatctgaagacagaggccgccctgccgcccgcgccgcaggacataagctacttacccaaaggcaaggtggccaacaccaacgtcacgcttcaggcgctgcgcggcaccaaggtggcggtgaaccagcgcagcgccgacgcgcccccggcgcccctgcccagcgccggcagcctgccctgggtcctcgagcagatcctgtgcctccagcagcagcagctgcagcagatccagctcacggagcagatccgggtgcaggtgaacatgtgggcctcgcacgccctccacgccggccacgcggccgactcgctgaagacgctcgggggccacgtgtcgcagcaggtgtcggcggccgtggccctgctcagccagaaggcggggagcgccgggctgcccctggacgccctgaagcccgccaagctacctcacgccaacgtcccttccgccggcggctccgcctccccggggctgccgcccttcgcgctgaagccggacgccacccgggtgctccccggcgtcctgtcgcgcctccccggggcgctgctcccccaggccccgggctctgtgctcttccagagccccttctccgcggtggccttagacccgtccaagaaaggcaaagggaagccaccgagtgtctccccggtggaggtcaaactcaaggacgaggctctctgccggcacaagtgtaagtactgtagcaaggtttttgggactgatagctccttgcagatccacctgcgctcccacaccggagagaggcccttcgtgtgctccgtgtgcggccaccgcttcaccaccaagggcaacctcaaggtgcacttccaccggcacccccaggtgaaggctaacccccagctgttggctgacttccacgacaagacggcggtgggcggcggcctccccttcgcgctggccggccccgcccccctcgaggaagccgggctctccctagacagcaaacccgcgctcgcggcggggacccccagtgtcctagggctagttcagaatctcccctcggggcctcaccccaaggacgccaaagggggcccgttgcccggcgacctgcagctcgggctgtctccggaaagcgaggatggctccatcctgcccggggtggggccgacccatccctcccccagggtcggaggcttcccagggggtgggccgcccgagccggggtcggagaccctgaagctgcagcggctggtggagaacatagacaaggccaccgccgaccccaacgagtgtctcatctgccaccgcgtcctcagctgccagagctcactcaagatgcactaccgcacgcacaccggggagcggcccttcccctgcaaggtctgcggccgcgccttctccaccaaaggcaacctgaagacgcacctggcggtgcaccgcagcagcgcgccgctgagggcgcagcactcctgcccgatctgccagaagaagttcaccaacgcggtcctgctgcagcagcacatccgcatgcacatgggcggccagatccccaacacgccgctgcccgagagcccctgcgagttcgcggggcccgagccctcggcggccggggagaacggcagccccggcgcgcccgctcacgacggggtcgtggaagccatcgacgtagacgaagcctgcccccaggaggcccccagcagctccttgagggtccccgggcccctcgccggcgtccacgcggcgtcccctaccccggggctggccacggcggcttcgctggatgtcccggtgaaggcggggctcgccgcgctcgtcctgcagcggcagggaagccgagaaaacgggtccgcggagagtgacggcctgaccaacgacgacgcctcctcggttatgggcgaccccgagtgcgccagccgaagcccagacgtcctggagagcacgtccttccaggcggtctcgccggcccatagccaggcggagagcgtcaagtccaagtctccggacaccgacggcaaaggggacggctcggagagcagccgcactgagatggaaggtcggagcagtgttccatcgacatttatccgagcccagccaacctatgtcaaagttgaagttcctggtgggtttgtgggtcccgcgaccatgtccccaggtatgacgcctctgttagcagcccagccccgacgacaggccaagcagcacggctgcacgaggtgcgggaagaacttctcgtcggccagcgcgcttcagatccacgagcggactcacaccggtgagaagccctttgtatgcaacatatgtggccgcgctttcaccaccaaaggcaacttgaaggtccattacatgacacacggggccaacaacagctcagcacgccgtggcaggaagctagccatcgagaacaccatggctctgttaggaacggacggaaagagggtccccgagatgtttcccaaggaaatcatggccccttcggtgaacgtggaccccgtcgtgtggaaccagtacgccaccatgctcaacggtggcctggccatgaagaccaacgagatctccgtaattcagagcggtggcatccccaccctccccgtgtccctgggggccagctccgtggtgaataacacggctgcctccaagatcgacggctcccagtctgccgctggtgccgaggtggagaagccagggactgccgacaacgtccccaaacaccagttcccgcacctcctggaagaaaacaagatcgcagtcagctaagccgcaggaggacgcgcctccgaggagcagtgcacgcagtgacctctctagagaactgtatctttttttgttctctctctttttttttaaggcccccatctcctccaggtttcttcctgatgtgcaaataatgtttacagttgtgaccacaacctcaggcacgtcttacaatcacaatgttgctatgctgctttgcaaaaaaagaaaaaagagaggaagaaaaaaaaattcttactaaaacaaatactagtatttttttaaattttggaagaaagcgggtcttgcaaagtagttttgttacttgcaacaaactatatacataaaaacctttgtacaacctcgagtaactgtttccaaagacggtcacctctttcaagttggaaggtattgaaccaccatggagaagacacctgttccacctgcttggggtcggggggtggcagggggtggggcggctgtatgcttgattctgtggggactggattcgggtaagtggacgtggtctgtcatctgtttcaggacccatttttgtattctcccaacccggcacccacttctcttttttttttttgaatgtatttttttttttggaaaaccaaggttgtagaattccagagtgtttttcagccttggaaccttaagtaggatgccctcaagtggactgattttagtccttagagagtcaatgtgtgtgttgctgcttatttaaatacagttcagttggagcctcaagagtgccaaggtcctctctacccttccagatgccgccttcttcctaaaaccaggagaggtgaccacctgagcagggaatctcaggtgatgtaatttagtccaccagtgcctgctctgttgaagagctcggtgtccattcttgaagaaacaaactcgtggaagggcatgttttcttattatatactaaaaacttatactaatttgtaccaacatttccttgattgtgacttacgccgagtaattatgaagatttttttttttcttctgagtattgcatgaaggctaccaagttggaacaggcaagtcctggatgtgaaagctttaatttatctacctcattcatgtgttatttttgtagctatagtctctattttcctattggatgaccgctgaagtgtttctagaccctatcgtaaacctaagagttgatgtattggtgggagcagctgaaagttcaagatgttgtgattcttttttttcttcttcttaatccccccttttgtatatgtgatattaagcagacgatgaagcgttcctcttgaagatttaacaagcaaggagaaagacccacatttctgggtgagacgtcttggcccctctgtttggaagagtggattgtggctcttctcaagtctgtctcttggtaattgcacctgagtttaggactccccagccccacaaatttttttgttttgttttgccaagttgtgcctttccttctggaattgtaagtgaacacaatgatagtacctgtttacactgtgaagcggatattgttacagaagatagaccagaggctttctcacttgttaagcttaataatgccttgtgaatgtatgatctacggagaaacccctgtagtttttacctgctgatgctgtctgttggagaataaattttgaatggttctttttttttcacca

SEQ ID NO: 85 - DNA, 3746 bp, Ovis aries
  source 1..3746: mol_type = other DNA
gcccctccctgatcaatgaggacttatttaaatgatctctgaggtcttggacccaggccaatcagctgtcagggctcatgataaatcgcaatgcattattgataataataattactgggacatgcgcgttccggccgaaggggggtaaatttcccaactccaggaatttgtggcggagtgggcaaataaccgcggctctccaggcgccccgatgctcgcaccatgtcgaggcgcaagcaggcgaagccccagcacatcaactcggaggaggatcagggcgagcggcagccgcagcagccggcccccgagtgtgcagatgcggccccagcggcgccggcggcgggggcgccgggtgctccagtgaactatcttggcggcggcggcagcagtggcagcgaggagccgaatggcgccggagcagggataaagcggccccggcgggaggagacccacgtctgcgagaaatgctgtgcggagttcttcagcttctccgacttcttggaccacaagaaaaattgcactaaaacgccccccgtgctcatcctgaaggacagtgaggggccgatgtcgtccgaagacttgccggggactgtgctcagctcgcagccgccgagcccgggccggagggagggccaccgggacgatggcagcggcggaggggcccccggggacgcgagggagaagccaggggcggagtctgtcctgtatctgaagacagaggccgccctgccgcccgcgccgcaggacataagctacttacccaaaggcaaggtggccaacaccaacgtcacgcttcaggcgctgcgcggcaccaaggtggcggtgaaccagcgcagcgccgacgcgcccccggcgcccctgcccagcgccggcagcctgccctgggtcctcgagcagatcctgtgcctccagcagcagcagctgcagcagatccagctcacggagcagatccgggtgcaggtgaacatgtgggcctcgcacgccctccacgccggccacgcggccgactcgctgaagacgctcgggggccacgtgtcgcagcaggtgtcggcggccgtggccctgctcagccagaaggcggggagcgccgggctgcccctggacgccctgaagcccgccaagctacctcacgccaacgtcccttccgccggcggctccgcctccccggggctgccgcccttcgcgctgaagccggacgccacccgggtgctccccggcgtcctgtcgcgcctccccggggcgctgctcccccaggccccgggctctgtgctcttccagagccccttctccgcggtggccttagacccgtccaagaaaggcaaagggaagccaccgagtgtctccccggtggaggtcaaactcaaggacgaggctctctgccggcacaagtgtcggagcagtgttccatcgacatttatccgagcccagccaacctatgtcaaagttgaagttcctggtgggtttgtgggtcccgcgaccatgtccccaggtatgacgcctctgttagcagcccagccccgacgacaggccaagcagcacggctgcacgaggtgcgggaagaacttctcgtcggccagcgcgcttcagatccacgagcggactcacaccggtgagaagccctttgtatgcaacatatgtggccgcgctttcaccaccaaaggcaacttgaaggtccattacatgacacacggggccaacaacagctcagcacgccgtggcaggaagctagccatcgagaacaccatggctctgttaggaacggacggaaagagggtccccgagatgtttcccaaggaaatcatggccccttcggtgaacgtggaccccgtcgtgtggaaccagtacgccaccatgctcaacggtggcctggccatgaagaccaacgagatctccgtaattcagagcggtggcatccccaccctccccgtgtccctgggggccagctccgtggtgaataacacggctgcctccaagatcgacggctcccagtctgccgctggtgccgaggtggagaagccagggactgccgacaacgtccccaaacaccagttcccgcacctcctggaagaaaacaagatcgcagtcagctaagccgcaggaggacgcgcctccgaggagcagtgcacgcagtgacctctctagagaactgtatctttttttgttctctctctttttttttaaggcccccatctcctccaggtttcttcctgatgtgcaaataatgtttacagttgtgaccacaacctcaggcacgtcttacaatcacaatgttgctatgctgctttgcaaaaaaagaaaaaagagaggaagaaaaaaaaattcttactaaaacaaatactagtatttttttaaattttggaagaaagcgggtcttgcaaagtagttttgttacttgcaacaaactatatacataaaaacctttgtacaacctcgagtaactgtttccaaagacggtcacctctttcaagttggaaggtattgaaccaccatggagaagacacctgttccacctgcttggggtcggggggtggcagggggtggggcggctgtatgcttgattctgtggggactggattcgggtaagtggacgtggtctgtcatctgtttcaggacccatttttgtattctcccaacccggcacccacttctcttttttttttttgaatgtatttttttttttggaaaaccaaggttgtagaattccagagtgtttttcagccttggaaccttaagtaggatgccctcaagtggactgattttagtccttagagagtcaatgtgtgtgttgctgcttatttaaatacagttcagttggagcctcaagagtgccaaggtcctctctacccttccagatgccgccttcttcctaaaaccaggagaggtgaccacctgagcagggaatctcaggtgatgtaatttagtccaccagtgcctgctctgttgaagagctcggtgtccattcttgaagaaacaaactcgtggaagggcatgttttcttattatatactaaaaacttatactaatttgtaccaacatttccttgattgtgacttacgccgagtaattatgaagatttttttttttcttctgagtattgcatgaaggctaccaagttggaacaggcaagtcctggatgtgaaagctttaatttatctacctcattcatgtgttatttttgtagctatagtctctattttcctattggatgaccgctgaagtgtttctagaccctatcgtaaacctaagagttgatgtattggtgggagcagctgaaagttcaagatgttgtgattcttttttttcttcttcttaatccccccttttgtatatgtgatattaagcagacgatgaagcgttcctcttgaagatttaacaagcaaggagaaagacccacatttctgggtgagacgtcttggcccctctgtttggaagagtggattgtggctcttctcaagtctgtctcttggtaattgcacctgagtttaggactccccagccccacaaatttttttgttttgttttgccaagttgtgcctttccttctggaattgtaagtgaacacaatgatagtacctgtttacactgtgaagcggatattgttacagaagatagaccagaggctttctcacttgttaagcttaataatgccttgtgaatgtatgatctacggagaaacccctgtagtttttacctgctgatgctgtctgttggagaataaattttgaatggttctttttttttcacca

SEQ ID NO: 86 - DNA, 2649 bp, Bos taurus
  source 1..2649: mol_type = other DNA
atgggcccttggagccgcagcctctctgcgctctgctgctgctgcaggtgtaatccgtggctctgccgggagccggagccctgcattcctggctttggtgctgagagttacacgttcaccgtgccccggcggaacttggagagagggcgagtcctaggcagagtgagttttgaaggatgtgctggcctaccaaggacagtctatgtttctgatgacacccgattcaaagtgcacacagatggcgtgcttacagtcagacgacctgtacaccttcatcgtccagagctaagttttcttgtccatgcctgggactccacccacaggaagctctccaccaaagtgacactggaggtatcagcgcaccaccaccaccaccacagtcatcatgactctccctctggaacccagacagaagtgctcacatttcctggcccccaccatggtctcaggagacagaagagagactgggttattcctcctatcagctgcccagaaaatgagaaaggcccatttcctaagtcgctggtccagatcaaatctaacaaggagaaagaaacccaagttttctacagcatcactggccaacgagctgatacaccccctgtcggtgtttttattattgaaagagaaacaggatggttaaaagtgacacagcctctggatagagaacagattgccaagtacattctcttctctcatgccgtgtcttcaaatggacaagccattgaagagcctatggagattgtgatcaccgtgaccgaccagaatgacaacaagccccagttcacccaggaggtcttcaaggcgtctgccctggaaggcgctcttccaggaacctctgtgatgcaggtcacggccacagatatagatgacgaggtgaacacctacaccgctgccatcggttacacaatcccagcccaagatcccatgctgccgcacaacaaaatgttcaccatcaacaaggaaacaggcgtcatcagtgtgctcaccaccgggctggaccgtgagagttttcccacatacaccctgatggtccaagcagcagaccttaacggcgaaggcttgagcacaactgcaacggccgtgatcacagtcttggacaccaatgataatgctcccagattcaacccaaccacgtacgtggggtcggtgcctgagaacgaggctaatgtggccatcaccacactcacagtgactgatgccgacgaccccaacaccccggcatgggaggctgtttacacagtattaaatgataacgagaagcaatttatcgtcgtcacagacccagtcaccaatgaaggcactctgaaaacagctaagggcttggattttgaggccaagcagcagtacatcctgtacgtggcagtgacaaatgtggccccctttgaagtcactctccccacttccacagccaccgtcactgtggatgtgatagatgtgaatgaagcccccatctttgtgcctcctcaaaagagagtggaagtgcccgaggactttggcgtgggcctggagatcacatcctatactgcccgggagccagacacatttatggaacagaagatcacgtatcggatttggagggacactgccaactggctggagattaatccagaaacgggtgccatttccactcgggctgagttggacagagaggatgtcgatcatgtgaagaacagcacgtacacggccctcattatagccactgacaatggttctccacctgccactgggacaggcaccctgctcttgttcctcgatgatgtgaatgacaatggccccgtaccagaaccccggaccatggacttctgccagaggaatcctgagcctcatatcatcaacatcaatgatcctgatctccctccgaacacctccccctttacagcagaactgacacatggggcgagtgtcaattggaccattgagtacaatgaccaagaacgtgagtctctgattttgaagccaaagaaaaccttagagctgggtgaccacaaaatcaatctcaagctcatagacaaccagaacaaagaccaggtgaccacacttgatgtgcacgtgtgtgactgtgatgggatcgtcagcaactgcaggaaggcacggcctgctgaagcaggattgcaagttcccgccatcctggggatccttggaggcatccttgcttttctgatccttattttgctgcttctgctacttgttcggaggagaagggtggtcaaagagcccttactgcccccagaagatgacacccgggacaatgtgtattactatgatgaagaaggaggtggagaagaagatcaggactttgacttgagccagttacataggggcctggatgctcggcctgaagtgactcgcaatgacgtggcaccaaccctcatgagtgtgccccagtaccgaccccgccctgccaatcctgatgaaattggaaactttattgatgaaaacctgaaggcagctgatagtgaccccactgccccaccctatgactctctgctggtgtttgattatgaaggaagtggttccgaagctgctactctgagctccctgaactcctcagagtcagaccaagaccaggactatgactacctgaatgaatggggcaatcgcttcaagaagctggcggacatgtatggaggcggcgaggacgactag

SEQ ID NO: 87 - DNA, 2649 bp, Bos taurus; identical to SEQ ID NO: 86
  source 1..2649: mol_type = other DNA

SEQ ID NO: 88 - DNA, 4284 bp, Sus scrofa
  source 1..4284: mol_type = other DNA
agtggcgtccggagcctcaaggcacctgtgagttcgcggaagtcctgccagactctagcccggcctagcccggccggaccctacccagtccccgcccggcccgccatgggccctccgtgccgcagtctctctgctctcctgctgctgttgcaggtctcatcgtggctctgccaagagccggagccctgcaatcctggctttggcgccaagagctacacgttcaccgtgccccggcggcacttggagagaggccgagtcctgggccgagtgagttttgaaggatgcaccggtctgccaaggacggtctatgtttctgatgacacccgattcaaagtgggcaccgacggtgtggttacagtcaaacggccatttcagcttcataacccggagattagttttctcatccacgcctgggactccacccgcaggaagctgtccaccaaggtgacactgaaggcggccacacaccaccatcaccaccaccgccatcaggactctccctctggcgcccacacagaagtgctcacgtttcccagctcccaccatggtctcagaagacagaaaagagactgggttatccctcccatcagctgcccagaaaatgagaaaggcccgtttcctaaggatctggttcagatcaaatctaacagagaaaaagaaaccaaggttttctacagcatcactggtcaaggagccgacacacctcccgtcggtgtgtttatgattgaaagagaaacagggtggctgaaggtgacggagcctctggatagagaagccattgccaagtacattctctactctcatgccgtgtcttccaacgggcaagccatcgaggagcccatggagattgtgatcaccgtgaccgatcagaacgacaacaagcccgagttcacccaggaggtcttcaggggctccgtcatggagggcgctcttccagggacctctgtgatgcaggtcacggccacggacaaagacgacgatgtgaacacctacaatgctgccattgcttacaccatcctcagccaggagcctaccctgcctcacaaccaaatgttcaccatcaacagtgacacaggagtcatcagtgtgctcaccactgggctggaccgggagagttttcccacgtacaccctggtgattcaggctgcagaccttcaaggtgacggcttaagcacgactgcaacagccatgatcacggtcactgacatcaacgacaacccacccatcttcaacccaacctcgtaccaggggtcggtgccggagaacgaggccaacgtggcaatcaccacactcacggtgaccgatggtgacgtcgccaataccccggcgtgggtggctgtttacacaatattaaatgacaacgagaagcaatttgttgtcatcacagacccagtaaccaatgaaggcgttttgaaaacagctaagggcttggattttgaagccaagcagcagtacatcctgtatgtggcagtgactaacgtggccccctttgaggtcaccctctctacttccacggccactgtcactgtggacgtgatagatgtgaatgaggcccccatcttcatccctgctcagaagagggtggaagtgcctgaggatttcggcgtgggcctggagatcacatcctacactgcccgggagccagacacgtttatggaacagaagatcacgtatcggatttggagggatgctgccaactggctggagattaatccggacaccggtgccattaccactcgggccgagctggacagagagaactttgagcatgtgaagaacagcacatacacggccctcattatagccactgacaatggttctccacccgctacgggcacaggcacccttctcctgttcctcgacgatgtgaatgacaatggccccatacctgaaccccgaaatatggacttctgccagaggaacccacagcctcatgtcatcaacatcaatgatccagatcttccccccaacacttctcccttcactgcagaactaacacatggggccagtgccaactggaccatcgagttcaacgaccgagaacatgaatctctcattttgaagccaaagaaaaccctcgagtttggcgactacaagatcaatctcaagctcacagacaaccagaacaaagaccaggtgaccaccttagatgtgcacgtatgcgactgtgagggggtggccaacagctgccggaaggccgggcctctggccgaagcaggattgcaagttcctgccatcctggggattctgggaggcatccttgcttttctgatcctgattctgctgctcctgctccttattcggaggagaagggtggtcaaagagcccttgctgcccccagaagatgacacccgggacaacgtttattactatgatgaagaaggaggtggagaagaggaccaggactttgacttgagccagttacacaggggcctggatgctcggcctgaagtgactcgtaatgatgtggcaccaaccctcctgagtgtgccccagtatcggccccgccctgccaatcctgatgaaattggaaactttattgatgaaaacctgaaggcagctgatagtgaccccactgccccaccttatgactctctgctggtgtttgattacgaaggcagtggttccgaagctgctagtctgagctccctgaactcctcggagtcggaccaagaccaggactatgactacctgaacgagtggggcaatcgcttcaagaagctggcggacatgtacggaggcggtgaggacgactaggggactccagaccgatggcagagatgggtccctacgctcatgtgatgggaaatgcagggacgatgttcctggcggttttccagctccccctccctttctggagaagagatgccgtgatctgtgatgcagttaggatagttgtcatttccactttctagatagaatctctgtgtgttagaacgatcttgacttgttccttcaatctttttttcctttcatcactctttacatggtgatgatgtccaaaagccccccaaattttagtatttccaaaggatagcttcagcctccgaaggttctgctggtaacttgcagatttccttaaaaggccttggctcatttttaaaaggaaggggtccatttccctgttgtatatatgagaaatgagtgtgcttttgtgcctcccatcatgtcagcagtgccccatgttcgaatatccacccttactcctgaattctattacatttctctgacacagaaattatcggacccttttaggaaaagaggttcatgtcttagtctggccacacactgagtgggtattgtgtactctttaatagtttgcctttttcatttcctgagtacttaacttgaaatggttacctatcctatttacttgttctgagtaagagtatttgttaagtgactgttattcatggggtactctggttctaagtagggagacgagaccagaaaagttgatgagttttcaggtgccactgaaatgtatccctaaaacagaagagagagagattccgggactcacaatagtgcctgaagtactgcagccaaagacagaagatggcttgtcacagcagaggggaaaggtgaaaagggggcttggagatgacaggggaagattgtccctgagcctggcaactgagctgactgaggccaaggatggttaaggtcgctccagccctgaaaattctgaaggagggaggaatctgaacatgtgtttcctaccttgtcctgtggttagtacccgaagcacagaatccccaagtgcctgcttttgttgatgtctgcagaaaatgctagctggtggggacacgtttgccctcaaattcccactgtgtgtaaaaacttgaaaatattttaaaaatgcgatttcccccctaggaaatggaagaaaaacacgccccaaaaggtgttagcaatgggaggcggaggtagggagaaccttaatttggatcttcttttagttgaaatacaaccttcaagaatgttttcacaaccacagacaagaactttaaattgcctccctatctctcaactgttttcagaagaaaaagccatccctgcaatcacctcacgggaattgtcttgatttttcagcagttgaaactctaatgtagttctgtgtagagaatgtcactgtagttttgcgagtatattgcgtgggtgctgataattttgtattttctttgggagtggaaaagaaaaacaattcaagctgaaaaaattattcttaaatattcatttttataaattttattaaagaattttggcaaaaaaaaaaaaaaa

SEQ ID NO: 89 - DNA, 4464 bp, Ovis aries
  source 1..4464: mol_type = other DNA
agtggcgtccggagcctcttagcacctgtgagttcgtagaagttctgccagactctagcccggcccaactcagctagatcctactcagcccgcgcccggcctgccatgggccctcggtgccgcaacctctctgcgctcctgctgctgctgcaggtctcatcgtggctctgccaggagccggagccctgcgtgcctggctttggcgctgagagttacacgttcaccgtgccccggcggaacttggagagagggcgagtcctgggcagagtgagttttgaaggatgtactggcctaccaaggacagtctatgtttctgacgacacccgattcaaagtgcacacagatggcctggttacagtcaaacggcctgtacaccttcatcgtccagagctaagttttcttgtccatgcctgggactccacccacaggaagctctccaccaaagtgacactgacggtatcagcgcaccaccatcaccaccaccgtcatcatgactctctctctggaactcagacggaagtgctcacatttcctggctcccaccatggtctccggagacagaagagagactgggtcattcctcccatcagctgcccagaaaatgagaaaggcccatttcctaagtcgctggtccagatcaaatctaacagggagaaagaaacccaagttttctacagcatcactggccaaggagctgacacaccccctgtcggtgtttttattattgaaagagaaacaggatggttaaaagtgacacagcctctggatagagaacacattgccaagtacattctcttctctcatgccgtatcttccaatggacaagccattgaagagcctatggagatcgtgatcacagtgaccgaccagaatgacaacaagccccagttcacccaggaggtcttcaaggggtctgtcctggaaggcgctcttccaggaacctctgtgatgcaggtcacagccacagatatagatgatgacgtgaacacccacaatgctgccatcggttacaaaatcctagcccaagagcccatgctgccacacaacaaaatgttcaccatcaacaaggaaacaggcgtcatcagtgtgctcaccaccgggctggaccgtgagagttttcccacatacaccctgatggtccaagcagcagaccttaatggcgaaggcttgagcacaactgcaacggccgtgatcacagtcctggacaccaatgataatgctcccagattcgacccgaccacgtacacggggtcagtgcctgagaacgaggctaacgtggccatcaccacactcacagtgaccgatgccgacgttcccaacaccccggcatgggaggctgtttacacagtattaaacgataatgagaaacaatttatcgtcatcacagacccagtcaccaatgaaggcactctgaaaacagctaagggcttggattttgaggccaagcagcagtacatcctgtacgtggcagtgacgaatgtggccccctttgaagtcactctccccacttccacagccacagtcactgtggatgtgatcgatgtgaatgaagcccccatctttgtgcctcctcaaaagaaagtggaagtgcctgaggactttggcgtaggcctggagatcacatcctataccgcccgggagccagacacatttatggaacagaagatcatgtatcggatttggagggacgctgccaactggctggagattaatccggaaacgggtgccatttccactcgggctgagttggacagagaggatgtagagcatgtgaagaacagcacgtacacagccctcatcatagccactgacaacggatctccacctgccactgggacaggcaccctgctcctgttcctcgatgatgtgaatgacaatggccccgtaccagaaccccgggccatggacttctgccagaggaatcccgagcctcacatcatcaacatcgtggatcctgatctccccccgaacacctcccccttcacagcagaactgacacatggggcgagtgtcaactggaccgttgagtacaatgaccaagaacgtgagtctctgattttgaagccaaagaaaaccttagaggtgggtgaccacaaaatcaatctcaagctcatagacaaccagaacaaagaccaggtgactacacttgatgtgcacgtatgtgactgtgatgggatcgtcagcaactgcaggaagcccaaggcctatgccgaagcaggattgcaagttcccgccatcctggggatcctcggaggcgtccttgcttttctgatccttattctgctgcttctgctacttgttcggaggagaagggtggtcaaagagcccttactgcccccagaagacgacacccgggacaatgtgtattactatgatgaagaaggaggtggagaagaagaccaggactttgacttgagccagttacataggggcctggatgctcgtcctgaagtgactcgcaatgacgtggcaccaaccctcatgagtgtgccccagtaccgaccccgccctgccaatcctgatgaaattggaaactttattgatgaaaacctgaaggcagctgatagtgaccccactgccccaccctatgactctctgctggtgtttgattatgaaggaagtggttccgaagctgctagtctgagctccctgaactcctcagagtcagaccaagaccaggactatgactacctgaatgaatggggcaatcgcttcaagaagctggcggacatgtatggaggcggcgaggatgactaagggactcaggactgatgggaagagagatgggtccctatactcgtgtgatggaaaatgtgggaaacatactcctggttattcgccagctcccttcactcgggatacgtttctggagaagagactgatcagtgatagcatagtcctgatttctactttatagatctaatctgtgtactagaacgatttagacttattccttgagtaatctttttcctttcatcacttttgacatggtgatgatgtccaaaatacccctaaattttaatacttcaaaataatagtttcagtctcagaaggttctgctagaaacttgcagattcccttaagggctttgtctcatttttaaaaggaaggggaggggtcagtttccctattgtgtgtgtgtgtatatatatatataaaagtatatatatatatatatatataaattgtgtgcttctgtccctcctgtcactgcactgatgtcctgtgttctgacatctgctcttactccttctgaattctattacatttctctggtgcagacattattgggctcttttagggaaagaggattatgtcttaggctggccatgtactgagtgggtattgtgtactcttaagaccttaaaggcttaacggtttttcatttttacgtaattgtgatgggcatctatccagttaacttgttctgagtaaggatgttggtcatgtgaatgtcattacaaggtactttggttctaaatagggaggctgggccagaaaaggtggtgagtttcaggcgccactgaactttttttttttttgatcgcactgcaggacatggggaatcttagttccctgactaggggtcagacctgcgtcccttaacaggggaatcgaggattcttaatcactggaccaccagggaaatcccactactaggcttctaatgtttatttatctctaaaacaagagtgatatattccaggacttagaatagtgcctaaagtgctgtagccaaagacagaagctggcaagagggcacagcagagggcaagtgtgaaaaatgggcttggagatggcaggggcagttgtcactaagcctggcaattgagccaactgaggctgaagacagttgaggtggctctacctccacccccctggaaattctgaactagaggaatctcaacatgtatttgccaggatccttggacccaaagcacagaatccccgagcccgcttttgttggtatttatagaaaatgctagctggtctggacacatttgccccaaatttcagctatgaaacttgagaatatttttcaaatccaatttgtttttcctttgcagaggaagaaaacactcactgaaagggtgttagcaaaggaaggtgggggtagggagtacattaatttggatcttcttttagttgaaatgcatcttttaagaaaattttgacaaccatagacaagaatttaatctgaattacttccctacctatcaactgtttttagaagaaaaggaacatctcccaaccacttcttagaactgtcttgatttttcagcaatttaaactctaatgtagttctacatagagaatgtcactatagttttgagtgtaatatgtgtatgggtgcagacaattttgtattttctttgggagtggaaaaggaaagcaattcaagctgcaaaaattattcttatttgtttttttataaattttattaaagaattttgttacaccatt

(19) United States
(12) Patent Application Publication
(10) Pub. No.: US 2026/0363218 A1
(43) Pub. Date: Nov. 19, 2026

(54) PRODUCTION OF LIVESTOCK ANIMALS FROM EMBRYONIC STEM CELLS

(71) Applicant: ABS Global, Inc., DeForest, WI (US)

(72) Inventors: Rebecca Lynn Krisher, Cottage Grove, WI (US); Benjamin Beaton, DeForest, WI (US); Jeff Betthauser, Windsor, WI (US); Joshua Benne, Madison, WI (US)

(73) Assignee: ABS Global, Inc., DeForest, WI (US)

(21) Appl. No.: 19/121,934

(22) PCT Filed: Oct. 16, 2023
(86) PCT No.: PCT/US2023/076955

§ 371 Date: Apr. 17, 2025

Related U.S. Application Data
(60) Provisional application No. 63/380,539, filed on Oct. 21, 2022.

(51) Int. Cl. A01K 67/027; C12N 5/00; C12N 5/073; C12N 5/0735; C12N 5/075; C12N 5/16; C12N 15/873

(52) U.S. Cl. CPC A01K 67/027; C12N 5/0604; C12N 5/0606; C12N 5/0609; C12N 5/16; C12N 5/562; C12N 15/873; A01K 2227/101; A01K 2227/103; A01K 2227/108; C12N 2501/415; C12N 2502/02; C12N 2510/00; C12N 2523/00

(57) ABSTRACT

Disclosed are methods of creating embryonic stem cells through nuclear transfer or embryo complementation. In general, methods of complementation include contacting embryonic stem cells with a host embryo. Host embryos can be wild-type embryos, tetraploid embryos, parthenogenetic embryos, trophoblastic vesicles, or embryos that have been gene edited to prevent them from making germ cells. Trophoblastic vesicles are typically made by ablating the inner cell mass of an embryo via heat shock, chemical treatment, laser ablation, or microdissection of the ICM. The embryonic stem cells can then be injected into the host embryo or fused with the host embryo by placing them in contact in culture. Once fused and matured, the resulting mosaic embryo is then implanted into a surrogate mother.

### CROSS REFERENCE TO RELATED APPLICATION

This application claims benefit of and priority to U.S. Provisional application 63/380,539 filed on Oct. 21, 2022. 63/380,539 is hereby incorporated by reference in its entirety.

### INCORPORATION OF THE SEQUENCE LISTING

The Sequence Listing, including the file named RB-43-2022-WO1-SEQLST.xml, is hereby incorporated by reference in its entirety.

### FIELD

This application applies to the field of livestock production and reproductive biology.

### BACKGROUND

As the world population increases, food production will also need to increase. However, recent estimates are that global livestock production is responsible for about 15% of the world's greenhouse gas emissions (Capper, J. L. and Cady, R. A., J. Anim. Sci., 2020, 98, skz291). One way to increase food production while minimizing environmental impact is through genetic progress—increased efficiency of animal germplasm based on genetic predictions of animal performance. For example, dairy production increased nearly 25% between 2007 and 2017, despite the number of cattle decreasing by the same amount—that is, 75% of the dairy cattle present in 2007 made 25% more milk in 2017 than in 2007 (Capper, J. L. and Cady, R. A., J. Anim. Sci., 2020, 98, skz291). This increase in efficiency resulted in only a 1% increase in greenhouse gases despite the much larger increase in milk production. (Greenhouse gas emissions will shortly start to decrease because the dairy industry as a whole has committed to Net Zero carbon emissions by 2050.) Genetic improvement has also driven increases in pork production; production increased from 12.1 billion pounds of pork in 1959 to 22.8 billion pounds of pork in 2009. In the same time period, land use for pork production decreased 78%, water use decreased 41%, and the carbon footprint of pork production decreased 35%.

Several reviews have postulated that increasing genetic progress in dairy germplasm can be expedited through in vitro breeding—most notably Goszczynski, D. E., et al., Biology of Reproduction, 2019, 100, 885-895. Conventional breeding requires gestating an embryo for 6 (pigs) or 9 (cattle) months after an embryo is created in vitro, then waiting another 7 months (pigs) to a year (cattle) for the animal to reach sexual maturity. One step in this process would be to produce bovine embryos and eventually calves from embryonic stem cells. Until recently (Bogliotti, Y. S., et al., Proc. Natl. Acad. Sci. USA, 2018, 115, 2090-2095), true embryonic stem cells were not available for livestock animals.

### SUMMARY

The present inventors disclose herein several methods of producing animals from true embryonic stem cells. In general, the present teachings disclose methods of embryo complementation that create mosaic embryos from host embryos that are fused with embryonic stem cells.

In some embodiments, the present teachings can encompass a method of generating an ungulate from an embryonic stem cell comprising contacting one or more embryonic stem cells with a host embryo to form a mosaic embryo. In various embodiments, the present teachings can encompass a method of generating an ungulate from an embryonic stem cell comprising contacting one or more embryonic stem cells with one or more host embryos to form one or more mosaic embryos. In various configurations, the ungulate can be a Bos taurus. In various configurations, the ungulate can be a pig. In some configurations, the host embryo can be a wild-type embryo, a tetraploid embryo, a parthenogenetic embryo, a trophoblastic vesicle, or a gene-edited embryo. In various configurations, the contacting can comprise injecting the embryonic stem cells into the host embryo. In various configurations, the contacting can comprise inserting the embryonic stem cells into the host embryo. In various configurations, the contacting can comprise coincubating the embryonic stem cells with the host embryo wherein the embryonic stem cells are physically touching the host embryo during coincubation. In various configurations, the contacting can comprise coincubating the one or more embryonic stem cells with the one or more host embryos wherein the embryonic stem cells are physically touching the host embryo during coincubation.

In various configurations, the host embryo can be at any stage between zygote and blastocyst stage. In various configurations, the host embryo can be a zygote, a two-cell stage embryo, a 4-cell stage embryo, an 8-cell stage embryo, a 16 to 32-cell stage embryo, a compacted morulae, or a blastocyst. In various configurations, the host embryo can be a zygote. In various configurations, the host embryo can be a two-cell stage embryo. In various configurations, the host embryo can be a 4-cell stage embryo. In various configurations, the host embryo can be an 8-cell stage embryo. In some configurations, the 8-cell stage embryo can be a compacted 8-cell stage embryo. In various configurations, the host embryo can be 16 to 32-cell stage embryo. In various configurations, the host embryo can be a compacted morulae stage embryo. In various configurations, the host embryo can be a blastocyst. In various configurations, the host embryo can be a gene edited embryo comprising an edit in Boule, Vasa, Dazl, Nanos1, Nanos2, Nanos3, PRDM1, PRDM14 (Blimp1), Kit, KitI, GDF9, BMP4, Nanog, Pou5f1 (Oct4), Sox2, Sall4, or Cdh1. In various configurations, the host embryo can comprise a deactivated Nanos2 gene. In various configurations, the deactivated Nanos2 gene can comprise a deletion of Nanos2.

In various configurations, a method of the present teachings can further comprise implanting the mosaic embryo into a surrogate mother. In some configurations, the ungulate can be a bovine, a porcine, or an ovine. In some configurations, the ungulate can be a kid, a piglet, a lamb, or a calf.

In some configurations, the embryonic stem cells can be incubated with a wnt inhibitor. In some configurations, the wnt inhibitor can be iwr-1, iwr-1-endo, iwp-2, Box5, iCRT3, sclerostin, dkk2, dkk1, LF3, CCT036477, FH535, cardamonin, IWP-L6, Wnt-C59, niclosamide, XAV-939, ICG-001, LGK-974, CP21R7, NCB-0846, PNU-74654, salinomycin, KY021 11, WIKI4, PRI-724, KYA1797K, 2,4-diamino-quinazoline, Ant 1.4Br, Ant 1.40, apicularen, bafilomycin, ETC-159, G007-LK, G244-LM, IWR, NSC668036, PKF1 15-584, pyrvinium, Quercetin, Shizokaol D, or BC2059. In various configurations, the wnt inhibitor can be iwr-1 or XAV-939. In various configurations, the wnt inhibitor can be iwr-1. In various configurations, the embryonic stem cells can have a stable karyotype. In various configurations, the one or more embryonic stem cells can be a colony of embryonic stem cells. In various configurations, the one or more embryonic stem cells can be a plurality of single embryonic stem cells.

In various configurations, the method can further comprise freezing the mosaic embryo. In some configurations, the freezing can be slow freezing or vitrification.

In various configurations, the host embryo can be a trophoblastic vesicle formed by ablating an inner cell mass (ICM) of an embryo. In some configurations, the ICM can be ablated by heat shock, phorbol 12-myristate 13-acetate treatment, laser ablation, or microdissection.

In various embodiments, a method of producing a bovine animal from bovine embryonic stem cells can comprise obtaining one or more host bovine embryos comprising a wild-type embryo, a tetraploid embryo, a parthenogenetic embryo, or a gene edited embryo; injecting one or more bovine embryonic stem cells into the one or more host bovine embryos to form a mosaic embryos; and implanting the one or more mosaic embryos into a surrogate mother.

In various embodiments, a method of producing a porcine animal from porcine embryonic stem cells can comprise: obtaining one or more host porcine embryos comprising a wild-type embryo, a tetraploid embryo, a parthenogenetic embryo, or a gene edited embryo; injecting one or more porcine embryonic stem cells into the one or more host porcine embryos to form one or more mosaic embryos; and implanting the one or more mosaic embryos into a surrogate mother.

In various embodiments, a method of generating an ungulate embryo can comprise: culturing one or more embryonic stem cells in a somatic cell media for 24-48 hours; inserting the one or more embryonic stem cells from the somatic cell media into one or more enucleated oocytes; fusing the one or more inserted embryonic stem cells with the one or more enucleated oocytes to form one or more embryos; activating the one or more fused embryos by injecting PLC Zeta RNA into the embryo; and transferring the one or more activated embryos into one or more surrogate mothers. In some configurations, the ungulate can be a porcine ungulate or a bovine ungulate. In various configurations, the ungulate is a piglet. In various configurations, the ungulate is a calf. In various embodiments, the present technology includes an ungulate animal made by any of the methods described herein. In various configurations, the ungulate is a piglet, a calf, or a lamb.

### DETAILED DESCRIPTION

In general, the methods of the present teachings are accomplished by merging one or more embryonic stem cells (ESC) with an activated oocyte or early stage embryo from another animal.

As used herein, a host embryo can be an early stage embryo or zygote to which embryonic stem cells can be introduced to form a mosaic embryo. A host embryo can be a wild-type embryo, a parthenogenetic embryo, a tetraploid embryo, an embryo carrying an edit in one or more genes that prevent the development of germ cells in the embryo, or a zygote that will mature into any of these embryos. In various configurations, the host embryo can be at any stage between zygote and blastocyst stage. In various configurations, the host embryo can be a zygote, a two-cell stage embryo, an 8-cell stage embryo, a compacting morulae, or a blastocyst. In various embodiments, the inner cell mass of a host embryo may be reduced or removed to reduce the contribution of the host embryo to the resulting animal.

As used herein, a somatic cell media can be any media used for cell culture that does not support pluripotency. Exemplary medias include bovine fibroblast culture media, or a base media such as MEM, DMEM, PRMI, or M199 supplemented with fetal calf serum.

### Embryonic Stem Cells

Embryonic stem cells (ESCs) can be generated, for example, by dissecting out the inner cell mass (ICM) of in vitro fertilized embryos, dissociating the cells, and then reseeding them onto a substrate. Alternatively, or in addition, whole embryos or blastocysts matured therefrom can be seeded onto a substrate to plate down to become embryonic stem cells. The zona pellucida can be removed via any method known in the art, including enzymatic digestion or laser assisted hatching.

### Inner Cell Mass Dissection

Inner cell masses can be isolated from blastocysts using a variety of techniques that are similar across species. ICM cells can be isolated using microsurgery, enzymatic digestion, immunosurgery, or mechanical separation.

Blastocysts can be placed in a suitable cell growth media such as HEPES-TL medium and then microsurgery can be performed via any technique known in the art, for example but without limitation using an ophthalmic scissors (see, for example, Gao, X. et al., Nature Cell Biology, 2019, 21, 687-699) or using a microblade connected to micromanipulation equipment (for example, a NT88-V3 high precision micromanipulator from Nikon/Narishige) attached to an inverted microscope (for example, a TE2000-U from Nikon; see, for example, Bogliotti, Y. S., et al., Proc. Natl. Acad. Sci. USA, 2018, 115, 2090-2095).

The zona pellucida of blastocysts can be removed by enzymatic digestion, such as via pronase, trypsin, or a combination thereof, until the trophoblasts begin to disperse in the microdrop. ICM cells can then be washed in stem cell culture medium and isolated with the aid of two fine needles and a pulled mouth micropipette (see, for example, Li, M., et al., Mol. Reprod. Dev., 2003, 65, 429-434).

Alternately, the ICM can be isolated using immunosurgery techniques by incubating the blastocyst in Tyrode's Solution (available from commercial sources such as Sigma-Aldrich) or pronase solutions to remove zona, followed by antibody and complement system mediated lysis of the trophectoderm (TE). Blastocysts can be incubated in stem cell culture medium supplemented with 10-20% anti-Bovine Serum for 30 min to 1 hour. The serum can be antibodies raised against bovine proteins from any suitable host, such as rabbit, goat, or rat. (For other ungulates, the serum should be raised against that species.) The serum can be washed out and then the cells can be incubated in stem cell culture medium supplemented with 10-20% complement serum for 30 min to 1 hour. Complement serum from various sources is commercially available, and sources include guinea pig complement. Once treated, ICMs can then be isolated from lysed trophoblast cells by pipetting and then washed multiple times in stem cell culture medium (see, for example, Hou, D. R., Sci. Rep., 2016, 6, 25838). For mechanical separation, zona-free blastocysts can be obtained by both spontaneous and artificial hatching processes and then washed three times. The ICMs can then be separated from the trophectoderm by pipetting with fine-pulled glass capillary pipettes in stem cell culture media (see, for example Jung, S. K., et al., J Vet Sci., 2014, 15, 519-528).

Alternatively, an embryonic stem cell culture can be initially established by plating a whole blastocyst. The zona pellucida can be removed via pronase or Tyrode's Solution (described supra) and then the entire blastocyst can be plated on feeder cells in media as described infra. The media can be formulated to promote ESC growth and not TE growth. So, the ESC will grow and the TE will have limited growth. Therefore, over time, the TE cells will be outcompeted and not be present in the culture.

Alternatively, the ICM can be isolated by laser assisted removal of the TE, which can be performed as follows: blastocysts can be secured by two holding pipettes with the ICM being positioned at 9 o'clock (relative to the field of view). Once adequate tension is established, approximately 10 infrared laser pulses (300 mW×1 ms, ZILOS-TK™, Hamilton Thorne Research, Beverly, MA) can be fired to split the blastocyst into two unequal portions—the smaller consisting of ICM, the larger consisting exclusively of trophoblast cells. The dissected ICM cells can be plated on feeder cells or directly into protein coated culture vessels.

### Embryonic Stem Cells

Once isolated, the isolated ICM cells are placed in a culture dish i) coated with an organic matrix such as fibronectin, Matrigel, or Vitronectin, ii) coated with decellularized extra cellular matrix (ECM; porcine gelatin, mixture of collagen IV, fibronectin, laminin and vitronectin) or iii) seeded with inactivated feeder cells. Suitable inactivated feeder cells include mitotically inactivated feeder cells such as gamma irradiated mouse embryonic fibroblast (MEF) or mitomycin treated MEF, human fetal muscle cells, human fetal fibroblasts, human adult fallopian tubal epithelial cells, human dermal fibroblasts, human amniotic mesenchymal cells, human amniotic epithelial cells, mouse bone marrow stromal cells, murine amniocytes, MEF SNL line, human amniocytes, human foreskin fibroblasts, human amniotic mesenchymal cells, pericellular matrix of decidua-derived mesenchymal cells, or another type of inactivated feeder cells. Inactivated MEF cells can be plated at 570,000 to 650,000 cells per well of a 6 well plate, 228,000 to 350,000 cells per well of a 12 well plate, or 100,000 to 118,000 cells per well of a 4 well plate. Plated MEF are good for 7 days. Cells are cultured in a basal media that is completely devoid of growth factors FGF2 and TGFβ.

Suitable media include CTFR Culture media (Ludwig, T. E., et al., Nature Methods, 2006, 637-646), ES culture media (Wu, X., et al., Sci Rep., 2016, 6, 28343), modified TeSR1 (e.g. lacking TGFP), TeSR1, N2B27, or E6 (available from Thermo-Fisher, catalog number A15164091). A modified N2B27 medium can comprise DMEM/F12 medium, Neurobasal Medium, 0.5×B-27 Supplement, 1×N2 supplement, 1×MEM Non-essential amino acid solution, 1×GLUTAMAX™ (Life Technologies Corporation, Carlsbad, CA), 100 U/ml penicillin-streptomycin, and 0.1 mM 2-Mercaptoethanol. The media can be supplemented with an inhibitor of wingless-integrated signaling (“WNT signaling”) and bFGF, FGF2, FGF4, or SUN 11602. A variety of signaling molecules and small molecules are known in the art to inhibit WNT signaling and are commercially available from suppliers such as Sigma-Aldrich (St. Louis, MO) and R&D systems (Minneapolis, MN). Such molecules include iwr-1, iwr-1-endo, iwp-2, Box5, iCRT3, sclerostin, dkk2, dkk1, LF3, CCT036477, FH535, cardamonin, IWP-L6, Wnt-C59, niclosamide, XAV-939, ICG-001, LGK-974, CP21R7, NCB-0846, PNU-74654, salinomycin, KY021 11, WIKI4, PRI-724, KYA1797K, 2,4-diamino-quinazoline, Ant 1.4Br, Ant 1.40, apicularen, bafilomycin, ETC-159, G007-LK, G244-LM, IWR, NSC668036, PKF1 15-584, pyrvinium, Quercetin, Shizokaol D, BC2059, and a combination thereof. A preferred embodiment is N2B27 medium supplemented with 20 ng/ml FGF and 2.5 μM IWR.

After 6-7 days in culture, ICM outgrowth can be passaged. This usually comprises dissociating the cells enzymatically (e.g. with pronase, trypsin, or recombinant trypsin) or mechanically (as discussed supra) and reseeding them onto newly prepared feeder cells with either i) culture medium and a Rho kinase (ROCK) inhibitor or ii) culture medium, FBS, and a serum replacement. Serum replacements are well known in the art and generally contain supplementary amino acids, anti-oxidants, and proteins such as insulin, transferrin, and lipid rich albumin. Serum replacements may also contain trace elements. Commercially available serum replacements include N2, B27, and KNOCKOUT® Serum Replacement (KSR; Gibco, Waltham, MA). A full discussion of serum replacements can be found, for example, in published application US20020076747. Suitable ROCK inhibitors include Y-27632, AS1892802, Fasudil hydrochloride, GSK 269962, GSK 429286, H 1152, Glyclyl-H 1152, HA 1100, OXA 06, RKI 1447, SB 772077B, SR 3677, PD-325901, and TC-S 7001. Passaged cells can be seeded into new wells containing the same media and feeder cells. Once established, media can be changed daily. The cells can be split 1:10 to 1:20 every 4-5 days. Cells can be maintained at 37 °C-38.5 °C in a humidified CO₂ incubator.

Cells prepared and maintained in this method are pluripotent and karyotypically normal (see, for example, Bogliotti, Y. S., et al., Proc. Natl. Acad. Sci. USA, 2018, 115, 2090-2095). Tests for pluripotency and methods of karyotyping are known in the art.

### Nuclear Transfer

In nuclear transfer, the nucleus of an ESC is transferred to an enucleated oocyte. Briefly, donor cells are dissociated by treatment with TrypLE enzyme for 5 min. Oocytes are harvested from either slaughterhouse-derived ovaries or from live animals by ultrasound-guided oocyte aspiration. The oocytes are matured in vitro and enucleated. A single cell from a selected embryonic stem cell culture is inserted into the perivitelline space of the enucleated oocyte using a 20-μm (internal diameter) glass pipet; this procedure is sometimes referred to as injecting the embryonic stem cell into the enucleated oocyte. Oocyte-cell fusion is induced using a square DC pulse generator. Fused oocytes can be activated using ionomycin followed by incubation in DMAP, then cultured under standard conditions. Alternatively, oocytes can be activated by injection of Phospholipase C zeta (PLCz) followed by incubation in media comprising Cytochalasin B. At 48 h after activation, noncleaved embryos are removed from culture and at 72 h after activation, the culture medium is optionally supplemented with serum and cultured for seven days before being recovered and implanted in synchronized recipients. Methods of embryo implantation are well known in the art. Offspring are born normally to the surrogate mother and are genetically identical to the embryonic stem cells.

### Nuclear Donor Cell Prep

The present inventors have shown that differentiated ESCs will produce more blastocysts upon somatic cell nuclear transfer. To that end, embryonic stem cells destined for nuclear transfer can be switched from embryonic stem cell maintenance media to bovine fibroblast culture media (BEFM). While growing in this media, the ES cells may lose their distinct dome shape and defined borders. Their morphology can flatten out and the cells can grow to contact inhibition which was previously not possible in the ES media. As discussed in detail in Example 12, this treatment can increase the number of blastocysts formed from nuclear transfer.

### Host Creation

In various embodiments, embryonic stem cells can be injected or otherwise introduced into a host to create a mosaic embryo. For embryos derived from trophoblastic vesicles, the cells from the host embryo can likely form the placenta and may not contribute to the final embryo. For embryos derived from parthenotes and tetraploids, these cells can have little to no contribution to the final embryo, as parthenogenetic embryos and tetraploid embryos can form a pregnancy but have yet to be shown to be brought to term. In some embodiments, the host can be a wild-type embryo. In some embodiments, the host embryos can be created using in vitro fertilization. Conventional, X-skewed, or Y-skewed bull semen can be used in IVF to generate host embryos. Standard IVF procedures can be used for each type of semen. In various embodiments, the host embryo can be created in vivo and obtained by flushing the animal.

In various embodiments, the host embryo can be non-viable or the host embryo can be unable or unlikely to contribute to the final animal. For example, the host embryo can be a parthenogenetic embryo or a tetraploid embryo. Alternatively, the host embryo can be a trophoblastic vesicle, the cells of which can form the placenta. Alternatively, the host embryo can carry one or more mutations in genes that are essential for formation of reproductive cells such as germ cells or gametes, thus being unable to form germ cells.

Alternatively, or in addition, the inner cell mass of an embryo can be ablated to either reduce or eliminate the contribution of the host to the mosaic embryo.

In general, in vitro production of livestock embryos is a three-step process involving oocyte maturation, oocyte fertilization and in vitro culture. Only 30-40% of such oocytes reach the blastocyst stage, at which point they can be transferred to a recipient or frozen for future use. IVF is a technique in which oocytes are fertilized in vitro. In an exemplary IVF procedure, oocytes are extracted from a donor animal by a method of aspiration from the ovary. Selected oocytes are then incubated for a period of about 24 hours; this is called the maturation period. After maturation, the eggs are fertilized about 18 to 22 hours after the co-culture has been made. The embryos stay in the medium until around the seventh day, when they are ready to be transferred. This technique has several main advantages over conventional in vivo embryo collection. With IVF, it is not necessary to superovulate the animals, nor is it necessary to synchronize them. This is a major breakthrough since the donor animals are not exposed to hormones that might compromise the reproductive soundness of the animals, and they can be worked without prior preparation time for the procedure. In some production systems, IVP efficiency can average about 30% of the oocytes harvested, although this quantity varies depending on the species, the breed, the donor animal, and also the mating. Further, animals can be aspirated every 14 days instead of every 60 days as in in vivo embryo collection. Finally, the animals can be harvested at a very young age, significantly improving genetic improvement rates by reducing the generation interval for the animals with specific desirable traits.

Embryo transfer (ET) technology allows producers to obtain multiple progenies at once from genetically superior females. Fertilized embryos can be recovered from females (also called embryo donors) of superior genetic merit by surgical or nonsurgical techniques. Alternately, oocytes can be harvested and then fertilized in vitro. The genetically superior embryos are then transferred to females (also called embryo recipients or surrogate mothers) of lesser genetic merit. In cattle, efficient techniques can recover fertilized embryos without surgery, but only one or sometimes two embryos are typically produced during each normal reproductive cycle. To increase the number of embryos that can be recovered from genetically superior females, the embryo donor can be treated with a hormone regimen to induce multiple ovulations, or superovulation.

In various embodiments, methods of the present teachings may be used with parthenogenetic embryos, which are oocytes that have been activated to undergo development in the absence of fertilization. Parthenogenetic embryos can be haploid or diploid, depending on whether or not the extrusion of the second polar body has been inhibited. Oocyte activation can be achieved by any method known in the art, including mechanical activation, electrical activation, and chemical activation. Chemical activation can be ionomycin activation, ethanol activation, hyaluronidase activation, Ca⁺² ionophores or chelators, cycloheximide, 6-diethylaminopurine (6-DMAP), or inhibitors of protein synthesis. Methods of generating parthenogenetic embryos are discussed in U.S. Pat. Nos. 5,843,754, 10190093, Tiziana A. L., et al., Biology of Reproduction, 72, 2005, 1218-1223, and Kharche, S. D. and Birade, H. S., Advances in Bioscience and Biotechnology, 2013, 4, Article ID: 28406.

In various embodiments, the methods of the present teachings can be used with tetraploid embryos. Tetraploid embryos can be made via electrofusion. Briefly, 2 cell embryos can be washed in a solution of 0.29 M D-mannitol, 0.1 mM magnesium sulfate, 0.05 mM calcium chloride, and 0.5% BSA. The embryos can then be placed in a fusion chamber and pulsed twice with 1.0 kV/cm for 25 μseconds. Embryos that fuse within one hour can be used for aggregation with ESCs. Alternatively, tetraploid embryos can be produced by chemically inhibiting cleavage and polar body extrusion in embryos using chemicals such as cytochalasin B. One such method would comprise activating an oocyte to make a parthenote and then treating the parthenogenetic embryo with a chemical to inhibit extrusion of both the first and second polar bodies.

In various embodiments, the methods of the present teachings can include ablation of the inner cell mass of an embryo to reduce the contribution of the host to the mosaic embryo created from fusion of the host with embryonic stem cells. These techniques can form a trophoblastic vesicle. In some configurations, the ablation can be via heat shock, treatment with phorbol 12-myristate 13-acetate (PMA), laser ablation, or microdissection. Embryos with ICMs ablated by any of these methods can become trophoblastic vesicles. For heat shock, Day 7 blastocysts can be heat treated for 15 minutes in 100 μL of media at 44 or 45 °C and then returned to culture conditions. The blastocysts typically collapse after the heat shock, but blastocysts that re-expand after overnight culture can show a reduction in the ICM cell count by >50% while the trophoblast cells appear unaffected. (See, for example, Modliński, J., et al., J. Animal and Feed Sci., 2004, 13, 197-204 for more detailed protocols.)

For ablation using PMA, 2-cell embryos can be cultured in media containing PMA (1-3 ng/ml) for 24-72 hours and then further cultured in embryo culture media. Blastocysts at Day 7 can be assessed for ICM contribution. (See, for example, Modliński, J., et al., J. Animal and Feed Sci., 2004, 13, 197-204.) Using these methods, blastocysts can be absent an organized ICM and may be useful to determine embryonic stem cells' ability to populate/derive their own ICM following injection.

For laser ablation, a XYRCOS® laser (Hamilton Thorne, Beverly, MA) set to 100% power with a 500 μsecond pulse can be fired across the ICM 5 times. This typically results in a collapsed blastocyst that re-expands after culture, thus reducing the ICM cell number by roughly 50%.

For microdissection, blastocysts can be micromanipulated with a microtool holder and embryo splitting blade (Shearer Precision Products) on an inverted microscope using a manipulation plate containing microdrops of TLHepes. The blastocyst can be held by slight suction of the holder and an embryo splitting blade can be used to press down on the blastocyst thereby cutting the embryo at the point where the blade contacts the bottom of the plate. The blastocyst can collapse, but may re-expand in culture. It is expected that this technique will yield a pure trophoblastic vesicle because the ICM is physically removed.

In various embodiments, embryonic stem cells of the present teachings can be introduced into the mosaic embryo through any method known in the art. In some configurations, the embryonic stem cells can be introduced through injection. In morula stage embryos, injection can comprise depositing cells into the interior of the morula. In blastocyst stage embryos, injection can comprise puncturing the blastocoel and depositing the embryonic stem cells into the blastocoel. The embryos comprising the embryonic stem cells can then be cultured in a 1:1 mix of embryo growth media and embryonic stem cell culture media. In some configurations, the embryonic stem cells introduced to the embryo can be a colony of embryonic stem cells.

Methods of implanting embryos into surrogate mothers for gestation are known in the art (see, for example, Tervit, H. R., et al., Theriogenology, 1980, 13, 63-71).

### Gene Editing

In some embodiments, the embryonic stem cells used in the present teachings can comprise a gene having a precise gene edit. Many gene editing methods require a guide nucleic acid molecule. A guide nucleic acid molecule is one that directs a nuclease to the specific cut site in the genome, whether via use of a binding domain, recognition domains, guide RNAs or other mechanisms. The guide nucleic acid molecule can be introduced into the cell under conditions appropriate for operation of the guide nucleic acid molecule in directing cleavage to the target locus. A person of skill in the art will have available a number of methods that may be used, the most common utilizing a nuclease to cleave the target region of the gene, along with sequences which will recognize sequences at the target locus and direct cleavage to the locus. Any nuclease that can cleave the phosphodiester bond of a polynucleotide chain may be used in the methods described here. By way of example without limitation, available systems include those utilizing site specific nucleases (SSN) such as ZFNs (Zinc finger nuclease), (Whyte, J. J., et al., Mol. Reprod. Dev., 2011, 78, 2; Whyte, J. J. and Prather, R. S., et al., J. Anim. Sci., 2012, 90, 1111-1117); TALENs (Transcription activator-like effector nucleases) (Carlson, D. F. et al., Proc. Natl. Acad. Sci. USA, 2012, 109, 17382-17387; Tan, W., et al., Proc. Natl. Acad. Sci. USA, 2013, 110, 16526-16531; Lillico, S. G., et al., Scientific Reports, 2013, 3, 2847), and the CRISPR (Clustered regularly interspaced short palindromic repeats)-associated (Cas) nuclease system (Hai, T., et al., Cell Res., 2014, 24, 372-375) that have permitted editing of animal genomes such as cattle or pig genomes with relative ease. Meganucleases have been used for targeting donor polynucleotides into a specific chromosomal location as described in Puchta, H., et al., Proc. Natl. Acad. Sci. USA, 1996, 93:5055-5060. ZFNs work with proteins or domains of proteins binding to a binding domain having a stabilized structure as a result of use a zinc ion. TALENs utilize domains with repeats of amino acids which can specifically recognize a base pair in a DNA sequence. For a discussion of both systems see U.S. Pat. No. 8,697,853, by Voytas, D. F., et al., incorporated herein by reference in its entirety. These systems utilize enzymes prepared for each target sequence.

Exemplary target genes include Boule, Vasa, Dazl, Nanos1, Nanos2, Nanos3, PRDM1, PRDM14 (Blimp1), Kit, KitI, or Gdnf9, BMP4, Nanog, Pou5f1 (Oct4), Sox2, Sall4, Cdh1, DCAF8, PPPIR12A, SLC16A3, UCP2, UCP3, TIGAR, AQP3, AQP7, HSPB8, PLAG1, KCNA6, NDUFA9, AKAP3, C5H12orf4, RAD51AP1, FGF6, TIGAR, CCND2, CSMD3, CHI3L2, GBP6, PPFIBP1, REP15, CYP4F2, TIGD2, PYURF, SLC10A2, FCHSD2, ARHGEF17, RELT, PRDM2, KDM5B, PPP1R12A, ZFP36L2, CSPP1, NPC1L1, NUDCD3, ACSS1, FCHSD2, TMEM68, TGS1, LYN, XKR4, FOXA2, GBP2, GBP5, FGD6, URB1, EVA1C, PRLR, NANOS2, Deadend (Dnd), APAF1, SMC2, GART, TFB1M, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPPIR12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, TIGAR or ZBTB. In various configurations, exemplary target genes can be Boule, Vasa, Dazl, Nanos1, Nanos2, Nanos3, PRDM1, PRDM14 (Blimp1), Kit, KitI, Gdnf9, BMP4, Nanog, Pou5f1 (Oct4), Sox2, Sall4, or Cdh1. Exemplary sequences below and in the sequence listing have been extracted from GenBank; skilled persons will recognize that several of the sequences are mRNA sequences given in DNA format and will be able to convert as needed by referring to Genbank.

TABLE 1
Exemplary Target Genes

| Gene Name | Gene Function | Genbank Sequence(s) |
|---|---|---|
| Boule | Spermatogenesis | FJ623266 (SEQ ID NO:1), |
| | | EU050657 (SEQ ID NO: 2), |
| | | GU187435 (SEQ ID NO: 3), |
| | | GU187434 (SEQ ID NO: 4), |
| | | XM_005672047 (SEQ ID |
| | | NO: 5), XM_021075731 |
| | | (SEQ ID NO: 6), |
| | | XM_004004798 (SEQ ID |
| | | NO: 7) |
| Vasa (DDX4) | Spermatogenesis | JX437185 (SEQ ID NO: 8), |
| | | NM_001278643 (SEQ ID |
| | | NO: 9), NM_001278644 |
| | | (SEQ ID NO: 10), |
| | | NM_001007819 (SEQ ID |
| | | NO: 11) |
| Dazl | Spermatogenesis | NM_001081725 (SEQ ID |
| | | NO: 12), EF501823 (SEQ |
| | | ID NO: 13), DQ408765 |
| | | (SEQ ID NO: 14), |
| | | DQ408764 (SEQ ID NO: |
| | | 15), NM_001291682 (SEQ |
| | | ID NO: 16), |
| | | JF411068 (SEQ ID NO: 17), |
| | | KU705632 (SEQ ID NO: |
| | | 18), |
| | | XM_003358321 (SEQ ID |
| | | NO: 19), XM_042242847 |
| | | (SEQ ID NO: 20) |
| Nanos1 | Germ cell | XM_005225796 (SEQ ID |
| | survival | NO: 21), XM_001928298 |
| | | (SEQ ID NO: 22), |
| | | XM_004020346 (SEQ ID |
| | | NO: 23) |
| Nanos2 | Germ cell | JX985756 (SEQ ID NO: |
| | survival | 24), NM_001281904 (SEQ |
| | | ID NO: 25), |
| | | XM_003127232 (SEQ ID |
| | | NO: 26), XM_042232206 |
| | | (SEQ ID NO: 27) |
| Nanos3 | Germ cell | XR_003511972 (SEQ ID |
| | survival | NO: 28), MK950848 (SEQ |
| | | ID NO: 29), |
| | | XM_027969516 (SEQ ID |
| | | NO: 30), XM_015095791 |
| | | (SEQ ID NO: 31) |
| PRDM1 | Primordial | NM_001192936 (SEQ ID |
| | germ cell | NO: 32), |
| | specification | XM_005659340 (SEQ ID |
| | | NO: 33), XM_001925350 |
| | | (SEQ ID NO: 34), |
| | | XM_013992732 (SEQ ID |
| | | NO: 35), XM_005659342 |
| | | (SEQ ID NO: 36), |
| | | XM_021090447 (SEQ ID |
| | | NO: 37), XM_005659341 |
| | | (SEQ ID NO: 38), |
| | | XM_021090455 (SEQ ID |
| | | NO: 39), XM_021090461 |
| | | (SEQ ID NO: 40), |
| | | XM_012182705 (SEQ ID |
| | | NO: 41), XM_004011238 |
| | | (SEQ ID NO: 42) |
| PRDM14 | Primordial | NM_001191264 (SEQ ID |
| (Blimp1) | germ cell | NO: 43), XM_021090784 |
| | specification | (SEQ ID NO: 44), |
| | | XM_027973466 (SEQ ID |
| | | NO: 45), |
| Kit | spermatogonial | NM_001166484 (SEQ ID |
| | proliferation | NO: 46), NM_001044525 |
| | and meiosis | (SEQ ID NO:47), |
| | | AB250963 (SEQ ID NO: |
| | | 48), NM_001308594 (SEQ |
| | | ID NO: 49) |
| Gdnf9 | Germ cell | NM_174681.2 (SEQ ID NO: |
| | specification | 50), GQ922451.1 (SEQ ID |
| | | NO: 51), NM_001001909.1 |
| | | (SEQ ID NO: 52), |
| | | HQ687750.1 (SEQ ID NO: |
| | | 53) |
| BMP4 | Germ cell | NM_001045877 (SEQ ID |
| | specification | NO: 54), NM_001101031 |
| | | (SEQ ID NO: 55), |
| | | EU549864 (SEQ ID NO: |
| | | 56), EU026433 (SEQ ID |
| | | NO: 57), NM_001110277 |
| | | (SEQ ID NO: 58), |
| | | ON933955 (SEQ ID NO: |
| | | 59) |
| Nanog | Germ cell | NM_001025344 (SEQ ID |
| | maintenance | NO: 60), DQ069776 (SEQ |
| | | ID NO: 61), |
| | | EF522119 (SEQ ID NO: |
| | | 62), NM_001129971 (SEQ |
| | | ID NO: 63), |
| | | XM_004006901 (SEQ ID |
| | | NO: 64), XM_015094959 |
| | | (SEQ ID NO: 65) |
| Pou5f1 | Germ cell | AH007191 (SEQ ID NO: |
| (Oct 4) | specification | 66), NM_174580 (SEQ ID |
| | | NO: 67), NM_001025344 |
| | | (SEQ ID NO: 68), |
| | | NM_001113060 (SEQ ID |
| | | NO: 69), KJ023672 (SEQ |
| | | ID NO: 70), KJ023671 |
| | | (SEQ ID NO: 71), |
| | | KT593924 (SEQ ID NO: |
| | | 72), XM_004018968 (SEQ |
| | | ID NO:73), XM_042237451 |
| | | (SEQ ID NO: 74) |
| Sox2 | Germ cell | NM_001105463 (SEQ ID |
| | specification | NO: 75), BC133458 (SEQ |
| | | ID NO: 76), |
| | | NM_001123197 (SEQ ID |
| | | NO: 77), EU519824 (SEQ |
| | | ID NO: 78), |
| | | NM_001318074 (SEQ ID |
| | | NO: 79), KT593925 (SEQ |
| | | ID NO: 80), X96997 (SEQ |
| | | ID NO: 81) |
| Sall4 | Formation of | NM_001192836 (SEQ ID |
| | spermatogonial | NO: 82), NM_001114673 |
| | stem cells | (SEQ ID NO: 83), |
| | | XM_027977337 (SEQ ID |
| | | NO: 84), XM_027977338 |
| | | (SEQ ID NO: 85) |
| Cdh1 | Germ cell | AY508164 (SEQ ID NO: |
| | formation/maintenance | 86), NM_001002763 (SEQ |
| | | ID NO: 87), |
| | | NM_001163060 (SEQ ID |
| | | NO: 88), XM_027978118 |
| | | (SEQ ID NO: 90) |

### EXAMPLES

The present teachings including descriptions provided in the Examples that are not intended to limit the scope of any claim or embodiment. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example illustrates generation of tetraploid host embryos for embryo complementation.

Tetraploid host embryos were produced by fusing 2-cell embryo blastomeres together. At approximately 30 hours after in vitro fertilization, the fertilized embryos began reaching the 2-cell stage of embryo development. At this stage, the 2-cell embryos were fused by placing them into a 0.5 mm fusion chamber containing mannitol fusion media (0.3M mannitol, 0.05 mM calcium chloride, 0.1 mM magnesium sulfate, and 0.1% bovine serum albumin (BSA)-fatty acid free (Sigma Aldrich)). A square wave DC pulse was delivered to the embryos to induce fusion of the individual blastomeres (BTX ECM 2001, two 25 usec pulses of 50V). The embryos were washed in TL Hepes (ABT360, LLC) containing 3 mg/mL BSA and fusion was assessed at 1 hour post fusion pulse. Typically, about 70-80% of the embryos fused and appeared as a one-cell embryo.

Fused 2-cell embryos were cultured for 12-14 hours in embryo culture media (BO-IVC, IVF Bioscience) and further cleavage was assessed. Fused 2-cell embryos that were 2-cells again at 12-14 hours post fusion tended to be tetraploid and were separated and cultured until they were used as host embryos for ES cell injection. Fused 2-cell embryos that appeared as 3- or 4-cell embryos at 12-14 hours post fusion were typically diploid or mixoploid (some cells were tetraploid, others were diploid) and were not used as host embryos.

### Example 2

This example illustrates the construction of Nanos2 gene edited host embryos.

Nanos2 host embryos were generated from zygotes fertilized with Y-skewed semen. At 16-18 hours post insemination, the selected sgRNPs (targeted to remove the single exon of bovine Nanos2) were injected into presumptive zygotes. Injections were performed on an inverted microscope (Nikon Eclipse Ti2) with micromanipulators (Narishige). Drops of TL Hepes were placed on a 100 mm petri dish and covered with mineral oil (Sigma Aldrich). Injection was performed by placing zygotes in the drops of TL Hepes and the sgRNP was injected by loading a pulled capillary tube (Sutter Instruments) with the editing reagent, aligning the capillary tube and pointing toward the zygote and then inserting the capillary into the zygote until the tool was inside the zygote cytoplasm. Once inside, pressure from a FEMTOJET® 4i microinjector (Eppendorf SE, Hamburg, Germany) (Pi: 200 hpa, Ti: 0.25, Pc: 15 hpa) injected the editing reagent into the zygote. The edited zygotes were cultured in embryo culture media until Day 6 of development (typically blastocyst stage).

### Example 3

This example illustrates the preparation of parthenogenetic host embryos.

Parthenogenetic host embryos were produced by activating mature oocytes. After about 24 hours of oocyte maturation, oocytes were stripped of their cumulus cells by vortexing in small amounts of media. Oocytes that were stripped of cumulus were activated by incubation for 4 minutes in 5 μM ionomycin (Sigma Aldrich) in TL Hepes containing 1 mg/mL BSA followed by washing for 5 minutes in TL Hepes containing 30 mg/mL BSA. After activation with ionomycin, oocytes were cultured in BO-IVC containing 2 mM 6-DMAP (Sigma Aldrich) for 4 hours. After 4 hours in DMAP, oocytes were washed for at least 5 minutes in TL Hepes and then cultured as embryos in BO-IVC. They were used as host embryos at the desired age/stage.

### Example 4

This example illustrates the preparation of ES cells for embryo complementation.

ES cell cultures that had grown into visible colonies were incubated with a thin film of ReLeSR™ (StemCell Technologies, Cambridge, MA). After about 5 minutes, 1 mL of N2B27 medium supplemented with 1% BSA (Soto, D. A., et al., Sci. Rep., 2021, 11, 11045) was added to the well and the plate was tapped several times to release ES cell colonies. The media and loose colonies were aspirated and placed into a microcentrifuge tube and centrifuged lightly to pellet the colonies. The supernatant was aspirated and replaced with about 50-100 μL TL Hepes. Some of the cells were distributed by pipette to designated manipulation plate drops for injection into host embryos.

### Example 5

This example illustrates ES cell injection into host embryos.

Morulae and blastocyst stage embryos were used as host embryos. On an inverted microscope equipped with micromanipulators and microtools, embryos were manipulated in drops of TL Hepes covered with mineral oil. A glass microtool holder was attached to an Eppendorf CELLTRAM® (Eppendorf SE, Hamburg, Germany) manual microinjector to control the host embryo. Another glass microtool (~20 μm diameter opening) was attached to another Eppendorf CELLTRAM® opposite the holder to facilitate aspiration and injection of the ES cell clumps into the host embryo.

ES cell clumps were aspirated into the microtool. Then, while holding a host blastocyst with the holder under slight suction, the host blastocyst was aligned with the inner cell mass (ICM) area being suctioned to the holder. A laser (RI Saturn 5, Cooper Surgical) was set to single pulse of 100% power and 500 μs was directed to produce a hole in the zona pellucida and sometimes another pulse to generate a weak spot in the expanding trophoblast. This typically induces the blastocyst to collapse. While the blastocyst was collapsing, the injector tool was inserted into the weak spot on the embryo and a clump of ~20 cells was injected and deposited into the embryo. The injection tool was removed and the process was repeated until all embryos had been complemented with ES cells.

When using a morulae stage host embryo, the same process was followed except there was no need to fire a second laser pulse to enter the embryo, the injector tool was simply inserted into the embryo mass and used to deposit the ES cells into the center of the embryo.

### Example 6

This example illustrates EC embryo culture of embryos complemented as discussed in Examples 1-5.

Complemented embryos were cultured in a 1:1 mix of embryo culture media and N2B27 medium supplemented with 1% BSA, 20 ng/ml FGF2 and 2.5 μM IWR-1 (Soto, D. A., et al., Sci. Rep., 2021, 11, 11045) until embryo transfer into recipient cattle. Alternately, embryo culture media alone can be utilized.

### Example 7

This example illustrates ES cell co-incubation with host embryos.

Host embryos will be co-incubated with ES cell clumps in microwell dishes to facilitate the uptake of ES cells into the host embryo. The zona pellucida will be removed from the host embryo via short incubation in Acidified Tyrode's solution (Sigma Aldrich) and then washed several times in TL Hepes. ES cells will be prepared by picking single colonies directly from culture plate using a 135-175 μm pipette (Stripper Tip, Origio). In a microwell dish (EmbryoSlide, Vitrolife) containing EC embryo culture media, the embryo and ES cells will be co-incubated in a microwell so the two bodies will be in contact with each other. The co-incubation will be assessed after a few hours and the microwells that contained a single body will be used for embryo transfer.

### Example 8

This example illustrates heat shocking blastocysts to create hosts that are trophoblastic vesicles.

Day 7 blastocysts were heat treated for 15 minutes in 100 μL of media at 44 or 45 °C and returned to culture conditions. It the blastocysts collapsed after the heat shock but then re-expanded after overnight culture; these blastocysts showed a reduction in the inner cell mass cell count by >50% while the trophoblast cells were unaffected. These trophoblastic vesicles will be complemented as described in Example 4 or 5 and then transferred into a surrogate mother.

### Example 9

This example illustrates use of PMA (phorbol 12-myristate 13-acetate) to create trophoblastic vesicles.

2-cell embryos were cultured in media containing PMA (1-3 ng/ml) for 24-72 hours and then further cultured in embryo culture media. Blastocysts at Day 7 were assessed for ICM contribution. Several blastocysts were absent an organized ICM. It is expected that blastocysts prepared in this manner will be useful to determine ES cell ability to populate/derive their own ICM following injection. These trophoblastic vesicles will be complemented as discussed in Example 4 or 5 and then transferred into a surrogate mother.

### Example 10

This example illustrates laser ablation of the ICM to create trophoblastic vesicles.

A XYRCOS® laser (Hamilton Thorne) set at 100% power, 500 μsecond pulse was used to fire 5 laser shots across the ICM. This killed a majority of ICM cells and allowed ES cell complementation (see Examples 4 and 5) to re-populate the ICM. These trophoblastic vesicles were complemented as discussed in Example 4 or 5 and then transferred into a surrogate mother.

### Example 11

This example illustrates microdissection of the ICM to create trophoblastic vesicles.

On an inverted microscope, a manipulation plate containing microdrops of TLHepes, blastocysts will be micromanipulated with a microtool holder and embryo splitting blade (Shearer Precision Products). The blastocyst will be held by slight suction of the holder and an embryo splitting blade will be used to press down on the blastocyst thereby cutting the embryo at the point where the blade contacts the bottom of the plate. It is expected that this procedure will allow for complete removal of the ICM, allowing for a pure trophoblastic vesicle. These trophoblastic vesicles will be complemented as discussed in Example 4 or 5 and then transferred into a surrogate mother.

### Example 12

This example illustrates utilizing an intermediate cell type to generate animals from bovine embryonic stem cells.

Bovine ES cells can be pushed into an intermediate cell type that works well for the nuclear transfer (NT) procedure. Twenty-four to 48-hours prior to use as NT donor cells, the ES cell maintenance media is replaced with a bovine fibroblast culture media (BEFM).

TABLE 2
BEFM formulation

| | Catalog | Stock | Amount | |
|---|---|---|---|---|
| Component | Reference | Concentration | (250 mL) | |
| DMEM + | 10569010 | 1X | 215 | mL |
| Glutamax | (ThermoFisher) | | | |
| Fetal Bovine | SH30071.03 | | 30 | mL |
| Serum (not heat | Hyclone: | | | |
| inactivated) | GELifeScinces) | | | |
| 2-mercaptoethanol | M3148 (Sigma) | 14.3M | 1.75 | μL (100 μM) |
| MEM NEAA | 11140 (Life | 100X | 2.5 | mL (1X) |
| | Technologies) | | | |
| Penicillin- | 15140122 | 100X | 2.5 | mL (1X) |
| Streptomycin | ThermoFisher | | | |

While in BEFM, the ES cells lose their distinct dome shape and defined borders. Their morphology flattens out and the cells can grow to contact inhibition which was previously not possible in the ES media.

Initial comparisons of blastocyst development from donor cells in ES cell media versus donor cells cultured in BEFM for a day or two showed a significant increase in embryo development due to the BEFM treatment. Table 3 lists data consolidated from several replicates.

TABLE 3
Blastocyst Formation Following Nuclear Transfer

| ES | Blastocysts | | BEFM | Blastocysts | |
|---|---|---|---|---|---|
| cell | from ES | % | cell | from ES | % |
| NT | cell NT | Blastocysts | NT | cell NT | Blastocysts |
| 290 | 7 | 2.4% | 333 | 58 | 17.4% |

This example illustrates increased blastocyst formation in nuclear transfer of embryonic stem cells that have been allowed to differentiate.

### Example 13

This example illustrates chemical activation of oocytes for nuclear transfer embryo production.

Phospholipase C zeta (PLCz) has been shown to induce fertilization-like calcium oscillations when injected into bovine oocytes. (Ross, P. J., et. al., BMC Developmental Biology 2008, 8, 16). Complementary RNA (cRNA) was produced from a vector containing the full-length coding sequence for bovine PLCz provided to the present inventors from Rafael Fissore (University of Massachusetts). The present inventors grew and purified plasmid at ABS Global, Inc. and then sent plasmid preparation to Biosynthesis (Lewisville, TX) to have them generate cRNA, purify it and perform QC.

Approximately 20 picoliters of PLCz was injected into oocytes at a concentration of 1 μg/μL around 4 hours post fusion of NT donor cells and then cultured in embryo culture media containing 7.5 μg/mL Cytochalasin B for 4 hours. After Cytochalasin B incubation, embryos were washed in TL-HEPES and cultured for 7 days in embryo culture media.

All publications cited herein are hereby incorporated by reference, each in their entirety.

1. A method of generating an ungulate from an embryonic stem cell comprising contacting one or more embryonic stem cells with one or more host embryos to form one or more mosaic embryos.

2. The method according to claim 1, wherein the host embryo is a wild-type embryo, a tetraploid embryo, a parthenogenetic embryo, a trophoblastic vesicle, or a gene-edited embryo.

3. The method according to claim 1, wherein the contacting comprises injecting the embryonic stem cell into the host embryo.

4. The method according to claim 1, wherein the contacting comprises coincubating the one or more embryonic stem cells with the one or more host embryos wherein the embryonic stem cells are physically touching the host embryos during coincubation.

5. The method according to claim 1, wherein the host embryo is a zygote, a two-cell stage embryo, a blastocyst, or a compacted morulae.

6. (canceled)

7. (canceled)

8. (canceled)

9. The method according to claim 1, wherein the host embryo is a gene edited embryo comprising an edit in Boule, Vasa, Dazl, Nanos1, Nanos2, Nanos3, PRDM1, PRDM14, Kit, KitI, Gdnf9, BMP4, Nanog, Pou5f1 (Oct4), Sox2, Sall4, or Cdh1.

10. The method according to claim 1, wherein the host embryo comprises a deactivated Nanos2 gene.

11. The method according to claim 10, wherein the deactivated Nanos2 gene comprises a deletion.

12. The method according to claim 1, further comprising implanting the mosaic embryo into a surrogate mother.

13. The method according to claim 1, wherein the ungulate is a bovine, a porcine, or an ovine.

14. The method according to claim 1, wherein the ungulate is a kid, a piglet, a lamb, or a calf.

15. The method according to claim 1, wherein the embryonic stem cells are incubated with a wnt inhibitor.

16. The method according to claim 1, wherein the embryonic stem cells have a stable karyotype.

17. The method according to claim 1, wherein the one or more embryonic stem cells are a colony of embryonic stem cells.

18. The method according to claim 1, wherein the one or more embryonic stem cells are a plurality of single embryonic stem cells.

19. The method according to claim 1, further comprising freezing the mosaic embryo.

20. The method according to claim 1, wherein the host embryo is a trophoblastic vesicle formed by ablating an inner cell mass (ICM) of an embryo.

21. The method according to claim 1, wherein the ICM is ablated by heat shock, phorbol 12-myristate 13-acetate treatment, laser ablation or microdissection.

22. A method of producing a ungulate animal from ungulate embryonic stem cells comprising:
obtaining one or more host ungulate embryos comprising a wild-type embryo, a tetraploid embryo, a parthenogenetic embryo, or a gene edited embryo;
injecting one or more ungulate embryonic stem cells into the one or more host ungulate embryos to form a mosaic embryo; and
implanting one of the one or more mosaic embryos into a surrogate mother.

23. (canceled)

24. A method of generating an ungulate embryo comprising:
culturing one or more embryonic stem cells in a somatic cell media for 24-48 hours;
inserting the one or more embryonic stem cells from the somatic cell media into one or more enucleated oocytes;
fusing the one or more inserted embryonic stem cells with the one or more enucleated oocytes to form one or more embryos;
activating the one or more fused embryos and one or more enucleated oocytes by injecting PLC Zeta RNA into the embryo; and
transferring the one or more activated embryos into one or more surrogate mothers.

25. (canceled)

26. (canceled)

27. (canceled)

28. (canceled)

29. (canceled)
